(19) United States
(12) Patent Application Publication
(10) Pub. No.: US 2010/0278835 A1
(43) Pub. Date: Nov. 4, 2010

(54) NOVEL COMPOUNDS 660

(75) Inventors: Helen Blade, Macclesfield (GB); Heather Marie Jackson, Macclesfield (GB); Gary Peter Tomkinson, Macclesfield (GB); Elisa Ann Carron, London (GB); James Andrew Lumley, London (GB); Christopher John Pilkington, London (GB); Alexander James Floyd Thomas, London (GB); Justin Warne, London (GB)

Correspondence Address: ASTRAZENECA R&D BOSTON, 35 GATEHOUSE DRIVE, WALTHAM, MA, 02451-1215, US

(73) Assignees: ASTRAZENECA UK LIMITED, London (GB); ARROW THERAPEUTICS LIMITED, London (GB)

(21) Appl. No.: 12/720,094

(22) Filed: Mar. 9, 2010

Related U.S. Application Data
(60) Provisional application No. 61/158,906, filed on Mar. 10, 2009.

(51) Int. Cl. A61K 31/519; C07D 403/06; A61K 31/517; C07D 471/04; A61P 31/14; A61P 11/00; A61K 39/42

(52) U.S. Cl. 424/147.1; 544/284; 514/266.23; 544/279; 514/264.1

(57) ABSTRACT

The invention provides compounds of formula (I)

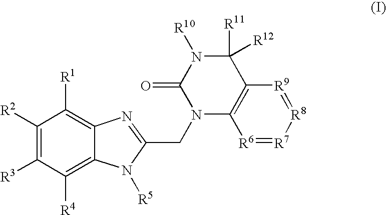

wherein R¹, R², R³, R⁴, R⁵, R⁶, R⁷, R⁸, R⁹, R¹⁰, R¹¹ and R¹² are as defined in the specification, and optical isomers, racemates and tautomers thereof, and pharmaceutically acceptable salts thereof; together with processes for their preparation, pharmaceutical compositions containing them and their use in therapy. The compounds are useful in the treatment of respiratory syncytial virus (RSV).

This application claims the benefit under 35 U.S.C. §119(e) of Application No. 61/158,906 filed 10 Mar. 2009.

The present invention relates to novel benzimidazole compounds, processes for their preparation, pharmaceutical compositions containing them and their use in therapy. In particular, the compounds are useful for the treatment of respiratory syncytial virus (RSV).

RSV was first identified in 1956 as the causal agent of chimpanzee coryza (Morris J A, Blount R E, Savage R E, Recovery of cytopathogenic agent from chimpanzees with coryza, Proc Soc Exp Biol Med 1956, 92:544-549) and was isolated from humans in 1957. After its isolation from children with pulmonary disease, followed by extensive characterisation, Chanock proposed the name ‘respiratory syncytial virus’ based on the in vitro and in vivo cellular observation that giant cells or syncytia are formed (Chanock R M, Roizman B, Myers R, Recovery from infants with respiratory illness of virus related to chimapanzee coryza agent (CCA): Isolation, properties and characterisation, Amer J Hyg 1957, 66:281-290). RSV is a negative-sense, single-stranded RNA virus of the Paramyxoviridae family.

RSV is readily transmitted by secretions from an infected person via surfaces or hand-to-hand transfer. Unlike influenza, it is not transmitted by small-particle aerosols. Following successful inoculation, the incubation period is between four and six days during which time the virus spreads from the nasopharynx to the lower respiratory tract by fusion of infected with uninfected cells and by sloughing of the necrotic epithelium. In infants, coupled with increased mucus secretion and oedema, this leads to mucus plugging causing hyper-inflation and collapse of distal lung tissue indicative of bronchiolitis (Handforth J, Friedland J S, Sharland M, Basic epidemiology and immunopathology of RSV in children, Paediatr Respir Rev. 2000 September; 1(3): 210-4). Hypoxia is common and the ability to feed is often impaired because of respiratory distress. In RSV pneumonia, inflammatory infiltration of the airways consists of mononuclear cells and is more generalised, with involvement of the bronchioles, bronchi and alveoli. The duration and degree of viral shedding has been found to correlate with the clinical signs and severity of disease.

RSV is the leading cause of serious respiratory tract infections in infants and young children throughout the world. The highest morbidity and mortality occurs in those born prematurely and for those with chronic lung or heart disease, although over 50% of infants hospitalised for RSV infection are otherwise healthy (Boyce T G, Mellen B G, Mitchel E F Jr, et al, Rates of hospitalization for respiratory syncytial virus infection among children in Medicaid, J. Pediatrics 2000, December; 137 (6) 865-870). Evidence suggests that severe infection in infancy often leads to several years of recurrent wheezing and is linked to the later development of asthma, further increasing the health care burden (Simoes E, Respiratory syncytial virus infection, Lancet 1999, 354:847-52). RSV is also a major cause of morbidity and mortality in the elderly and in immunocompromised children (Hall C B, Powell K R, MacDonald N E, et al, Respiratory viral infections in children with compromised immune function, N Engl J Med 1986, 315:77) and adults (Bowden R A, Respiratory Virus infections after Marrow Transplant: The Fred Hutchinson Cancer Research Centre experience, Am J Med 1997, 102 (3A):27-30) as well as those with chronic obstructive pulmonary disease (COPD) and congestive heart failure (CHF) (Greenberg S B, Allen M, Wilson J, Atmar R L, Respiratory viral infections in adults with and without chronic obstructive pulmonary disease, Am J Respir Crit Care Med 2000, July; 162(1): 167). Although immunocompetent adults less than 65 years of age are rarely hospitalised for RSV infection, a recent report concluded that the associated morbidity can result in appreciable cost for medical visits and absence from work (Hall C B, Long C E, Schnabel K C, Respiratory syncytial virus infections in previously healthy working adults, Clinical Infectious Diseases 2001, 33:792-6).

RSV has a seasonal incidence; it is highly predictable and occurs in the winters of both hemispheres, from September to May in Europe and North America, peaking in December and January, and can occur throughout the year in tropical countries. It affects >90% of infants and young children by the age of two years and as natural immunity is short-lived; many will be re-infected each year. As with influenza, in elderly people, RSV causes around 10% of winter hospitalisations with an associated mortality of 10%.

Current anti-RSV treatment involves the use of a monoclonal antibody to RSV, called palivizumab. Such use of palivizumab is a prophylactic, rather than therapeutic, treatment of RSV. Although this antibody is often effective, its use is restricted to preterm infants and infants at high risk. Indeed, its limited utility means that it is unavailable for many people in need of anti-RSV treatment. There is therefore an urgent need for effective alternatives to existing anti-RSV treatment.

Additionally, several compounds have been proposed as inhibitors of RSV, including benzimidazole-based compounds. For example, K D Combrink et al., Bioorganic & Medicinal Chemistry Letters, 17 (2007), 4784-4790 discloses the compound BMS-433771 and variants thereof:

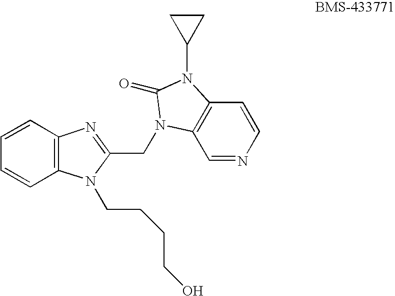

The variants of BMS-433771 disclosed in K D Combrink et al. include compounds containing 6,6-fused ring systems in place of the 5,6-fused ring system found in BMS-433771. However, K D Combrink et al. teaches that the intrinsic potency associated with the 6,6-fused ring systems is less than that for the 5,6-fused ring systems.

Further benzimidazole-based compounds are disclosed in WO-02/062290 and WO-03/053344. There is however no disclosure in any of K D Combrink et al., WO-02/062290 and WO-03/053344 of a benzimidazole-based compound containing a quinazolin-2-one or pyridopyrimidin-2-one substituent.

Surprisingly, we have now found that a select group of benzimidazole compounds possess improved thermodynamic solubility. It is believed that this improved thermodynamic solubility provides improved pharmacokinetic properties to the compounds and/or aids in their formulation for pharmaceutical uses.

In one aspect, the present invention provides a compound of formula (I), or a pharmaceutically acceptable salt thereof,

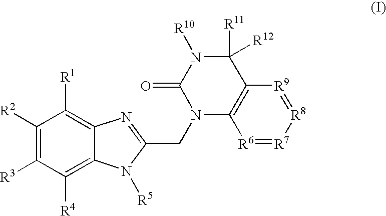

wherein:

R¹, R³ and R⁴ each independently represents H, C₁₋₆ alkyl or halogen;

R² represents H, CN, CH₂NH₂, CH₂NH(CH₂)₃NH₂, C(═NH)NH₂ or C(═NOH)NH₂;

R⁵ represents C₁₋₆ alkyl; said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ independently represent H, C₁₋₆ alkyl or C3-7 cycloalkyl; or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl;

R⁶, R⁷, R⁸ and R⁹ each independently represents CH, C—F, C—Cl, C—CF₃ or N;

R¹⁰ represents aryl, heteroaryl, C3-7 cycloalkyl or C₁₋₆ alkyl; said C₁₋₆ alkyl or C3-7 cycloalkyl being optionally substituted with one or more of aryl, C3-7 cycloalkyl, OR¹⁶, SR¹⁶, halogen or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰ wherein R²⁰ represents H or C1-6 alkyl; and

R¹¹ and R¹² each independently represents H or C1-6 alkyl.

In another aspect, the present invention provides a compound of formula (I), or a pharmaceutically acceptable salt thereof, wherein:

R¹, R³ and R⁴ each independently represents H, C₁₋₆ alkyl or halogen;

R² represents H, CN, CH₂NH₂, CH₂NH(CH₂)₃NH₂, C(═NH)NH₂ or C(═NOH)NH₂;

R⁵ represents C₁₋₆ alkyl; said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ independently represent H, C₁₋₆ alkyl or C3-7 cycloalkyl; or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl;

R⁶, R⁷, R⁸ and R⁹ each independently represents CH, C—F, C—Cl, C—CF₃ or N;

R¹⁰ represents aryl, heteroaryl, C3-7 cycloalkyl or C₁₋₆ alkyl; said C₁₋₆ alkyl being optionally substituted with one or more of aryl, C3-7 cycloalkyl, OR¹⁶, SR¹⁶, halogen or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰ wherein R²⁰ represents H or C1-6 alkyl; and

R¹¹ and R¹² each independently represents H or C1-6 alkyl.

In another aspect, the present invention provides a compound of formula (I), or a pharmaceutically acceptable salt thereof, wherein:

R¹, R³ and R⁴ each independently represents H, C₁₋₆ alkyl or halogen;

R² represents H, CN, CH₂NH₂, CH₂NH(CH₂)₃NH₂, C(═NH)NH₂ or C(═NOH)NH₂;

R⁵ represents C₁₋₆ alkyl; said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ independently represent H, C₁₋₆ alkyl or C3-7 cycloalkyl; or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl;

R⁶, R⁷, R⁸ and R⁹ each independently represents CH, C—F, C—Cl or N;

R¹⁰ represents aryl, heteroaryl, C3-7 cycloalkyl or C₁₋₆ alkyl; said C₁₋₆ alkyl being optionally substituted with one or more of aryl, C3-7 cycloalkyl, OR¹⁶, SR¹⁶, halogen or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰ wherein R²⁰ represents H or C1-6 alkyl; and

R¹¹ and R¹² each independently represents H or C1-6 alkyl.

In the context of the present application, an alkyl moiety may be linear or branched. However references to individual alkyl groups such as “propyl” are specific for the straight-chain version only and references to individual branched-chain alkyl groups such as “isopropyl” are specific for the branched-chain version only.

R¹, R³ and R⁴ each independently represents H or C1-6 alkyl (e.g. methyl, ethyl, 1-propyl, 2-propyl, n-butyl, iso-butyl, tert-butyl, pentyl or hexyl) or halogen (e.g. fluoro, chloro, bromo or iodo). In one embodiment, each of R¹, R³ and R⁴ independently represents H or C1-2 alkyl, particularly methyl. In another embodiment, each of R¹, R³ and R⁴ represents H.

In one embodiment, R² represents H, CH₂NH₂, CH₂NH(CH₂)₃NH₂, C(═NH)NH₂ or C(═NOH)NH₂. In another embodiment, R² represents CH₂NH₂, CH₂NH(CH₂)₃NH₂, C(═NH)NH₂ or C(═NOH)NH₂. In another embodiment, R² represents H. In another embodiment, R² represents CN. In another embodiment, R² represents CH₂NH₂. In another embodiment, R² represents CH₂NH(CH₂)₃NH₂. In another embodiment, R² represents C(═NH)NH₂. In another embodiment, R² represents C(═NOH)NH₂.

In one embodiment, at least one of R¹, R², R³ and R⁴ does not represent H. For example, in one embodiment at least R² does not represent H, such that R² represents CN, CH₂NH₂, CH₂NH(CH₂)₃NH₂, C(═NH)NH₂ or C(═NOH)NH₂, particularly CH₂NH₂, CH₂NH(CH₂)₃NH₂, C(═NH)NH₂ or C(═NOH)NH₂.

R⁵ represents C₁₋₆ alkyl (e.g. methyl, ethyl, 1-propyl, 2-propyl, n-butyl, iso-butyl, tert-butyl, pentyl, iso-pentyl or hexyl); said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ each independently represent H, C₁₋₆ alkyl or C3-7 cycloalkyl (e.g. cyclopropyl, cyclobutyl, cyclopentyl, cyclohexyl or cycloheptyl); or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl. In one embodiment, R⁵ represents C₁₋₆ alkyl (e.g. methyl, ethyl, 1-propyl, 2-propyl, n-butyl, iso-butyl, tert-butyl, pentyl, iso-pentyl or hexyl); said C₁₋₆ alkyl being optionally substituted with one or more of OR¹³ or CF₃, wherein R¹³ represents H or C₁₋₆ alkyl. In one embodiment, R⁵ represents C₁₋₆ alkyl (e.g. methyl, ethyl, 1-propyl, 2-propyl, n-butyl, iso-butyl, tert-butyl, pentyl, iso-pentyl or hexyl); said C₁₋₆ alkyl being optionally substituted with one or more of OH or CF₃, particularly CF₃.

In one embodiment, R⁵ represents iso-pentyl, 4-hydroxybutyl or 4,4,4-trifluorobutyl.

In one embodiment, R⁵ represents iso-pentyl or 4,4,4-trifluorobutyl.

In one embodiment, R⁵ represents C₁₋₆ alkyl, particularly iso-pentyl.

In another embodiment, R⁵ represents C₁₋₆ alkyl substituted by OH, particularly 4-hydroxybutyl.

In another embodiment, R⁵ represents C₁₋₆ alkyl substituted by CF₃, particularly 4,4,4-trifluorobutyl.

Examples of a 5 to 7 membered azacyclic ring optionally incorporating one further heteroatom selected from O, S and NR¹⁹ include pyrrolidine, piperidine, piperazine, N-alkylpiperazine, morpholine, thiomorpholine and perhydroazepine.

R⁶, R⁷, R⁸ and R⁹ each independently represents CH, C—F, C—Cl, C—CF₃ or N. In one embodiment, R⁶, R⁷, R⁸ and R⁹ each independently represents CH, C—F, C—Cl or N. In another embodiment, R⁶, R⁷, R⁸ and R⁹ each independently represents CH, C—F, C—CF₃ or N. In another embodiment, R⁶, R⁷, R⁸ and R⁹ each represents CH. In another embodiment R⁷, R⁸ and R⁹ each represents CH and R⁶ represents CH or N. In another embodiment R⁷, R⁸ and R⁹ each represents CH and R⁶ represents N. In another embodiment R⁶, R⁷ and R⁸ each represents CH and R⁹ represents CH, C—F or C—CF₃. In another embodiment R⁶, R⁷ and R⁸ each represents CH and R⁹ represents C—F. In another embodiment R⁶, R⁷ and R⁸ each represents CH and R⁹ represents C—CF₃.

R¹⁰ R represents aryl, heteroaryl, C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl, iso-pentyl or hexyl); said C₁₋₆ alkyl or C3-7 cycloalkyl being optionally substituted with one or more of aryl, C3-7 cycloalkyl, OR¹⁶, SR¹⁶, halogen (e.g. fluoro, chloro, bromo or iodo) or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰.

Examples of aryl include phenyl, naphthyl, indenyl, indanyl and tetrahydronaphthyl.

A suitable heteroaryl ring is, for example, an aromatic 5- or 6-membered monocyclic ring with up to 4 ring heteroatoms independently selected from oxygen, nitrogen and sulfur. Examples of a heteroaryl ring include pyridinyl, pyrimidinyl, pyridazinyl, pyrazinyl, triazinyl, oxazolyl, pyrazolyl, thienyl, isoxazolyl, isothiazolyl, thiadiazolyl, pyrrolyl, furanyl, thiazolyl, imidazolyl, triazolyl and tetrazolyl.

In one embodiment, R¹⁰ represents aryl, heteroaryl, C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl, iso-pentyl or hexyl); said C₁₋₆ alkyl is being optionally substituted with one or more of aryl, C3-7 cycloalkyl, OR¹⁶, SR¹⁶, halogen (e.g. fluoro, chloro, bromo or iodo) or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰.

In one embodiment, R¹⁰ represents C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl or hexyl); said C₁₋₆ alkyl or C3-7 cycloalkyl being optionally substituted with one or more of aryl, C3-7 cycloalkyl, OR¹⁶, SR¹⁶, halogen or NR¹⁷R¹⁸, wherein R¹⁶ represents C₁₋₆ alkyl and the group —NR¹⁷R¹⁸ together represents a 5 to 7 membered azacyclic ring optionally incorporating one further heteroatom selected from O, S and NR²⁰.

In one embodiment, R¹⁰ represents C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl or hexyl); said C₁₋₆ alkyl being optionally substituted with one or more of aryl, C3-7 cycloalkyl, OR¹⁶, SR¹⁶, halogen or NR¹⁷R¹⁸, wherein R¹⁶ represents C₁₋₆ alkyl and the group —NR¹⁷R¹⁸ together represents a 5 to 7 membered azacyclic ring optionally incorporating one further heteroatom selected from O, S and NR²⁰.

In one embodiment, R¹⁰ represents C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl or hexyl); said C₁₋₆ alkyl or C3-7 cycloalkyl being optionally substituted with one or more of aryl, C3-7 cycloalkyl, OR¹⁶, SR¹⁶ or NR¹⁷R¹⁸, wherein R¹⁶ represents C₁₋₆ alkyl and the group —NR¹⁷R¹⁸ together represents a 5 to 7 membered azacyclic ring optionally incorporating one further heteroatom selected from O, S and NR²⁰.

In one embodiment, R¹⁰ represents C3-7 cycloalkyl (e.g. cyclopropyl, cyclobutyl, cyclopentyl, cyclohexyl or cycloheptyl) or C₁₋₆ alkyl (e.g. methyl, ethyl, 1-propyl, 2-propyl, n-butyl, iso-butyl, tert-butyl, pentyl or hexyl); said C₁₋₆ alkyl being optionally substituted with one or more of aryl, C3-7 cycloalkyl, OR¹⁶, SR¹⁶ or NR¹⁷R¹⁸, wherein R¹⁶ represents C₁₋₆ alkyl and the group —NR¹⁷R¹⁸ together represents a 5 to 7 membered azacyclic ring optionally incorporating one further heteroatom selected from O, S and NR²⁰.

In one embodiment, R¹⁰ represents C1-6 alkyl, particularly C1-5 alkyl (e.g. methyl, 1-propyl, iso-butyl, tert-butyl or iso-pentyl).

In another embodiment, R¹⁰ represents C3-7 cycloalkyl, particularly cyclopropyl or cyclopentyl, more particularly cyclopropyl.

In another embodiment, R¹⁰ represents C1-6 alkyl substituted with one or more of aryl, C3-7 cycloalkyl, OR¹⁶, SR¹⁶, halogen or NR¹⁷R¹⁸. In another embodiment, R¹⁰ represents C1-6 alkyl (particularly C1-3 alkyl) substituted with one or more of aryl, C3-7 cycloalkyl, OR¹⁶, SR¹⁶ or NR¹⁷R¹⁸. In another embodiment, R¹⁰ represents C1-3 alkyl substituted with phenyl, cyclopropyl, cyclohexyl, OCH₃, SCH₃ or 1-pyrrolidinyl.

R¹¹ and R¹² each independently represents H or C1-6 alkyl (e.g. methyl, ethyl, 1-propyl, 2-propyl, n-butyl, iso-butyl, tert-butyl, pentyl or hexyl). In one embodiment, R¹¹ and R¹² each independently represents H or methyl. In another embodiment, R¹¹ and R¹² each independently represents H. In another embodiment, one of R¹¹ and R¹² represents H and the other of R¹¹ and R¹² represents methyl.

In one embodiment, each of R¹, R³ and R⁴ independently represents H or C1-2 alkyl, particularly methyl; R² represents H, CH₂NH₂, CH₂NH(CH₂)₃NH₂, C(═NH)NH₂ or C(═NOH)NH₂; R⁵ represents C₁₋₆ alkyl; said C₁₋₆ alkyl being optionally substituted with OR¹³ or CF₃ wherein R¹³ represents H; R⁶, R⁷, R⁸ and R⁹ each independently represents CH, C—F, C—CF₃ or N; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸, wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ independently represents H or C1-2 alkyl, particularly methyl; R² represents CH₂NH₂, CH₂NH(CH₂)₃NH₂, C(═NH)NH₂ or C(═NOH)NH₂; R⁵ represents C₁₋₆ alkyl; said C₁₋₆ alkyl being optionally substituted with OR¹³ or CF₃ wherein R¹³ represents H; R⁶, R⁷, R⁸ and R⁹ each independently represents CH, C—F, C—CF₃ or N; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸, wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ independently represents H or C1-2 alkyl, particularly methyl; R² represents CH₂NH₂, CH₂NH(CH₂)₃NH₂, C(═NH)NH₂ or C(═NOH)NH₂; R⁵ represents C₁₋₆ alkyl; said C₁₋₆ alkyl being optionally substituted with OR¹³ or CF₃; R⁶, R⁷, R⁸ and R⁹ each independently represents CH, C—F, C—Cl or N; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶, halogen or NR¹⁷R¹⁸; and R¹¹ and R¹² each independently represents H or methyl.

In one embodiment, each of R¹, R³ and R⁴ independently represents H or C1-2 alkyl, particularly methyl; R² represents CH₂NH₂; R⁵ represents C₁₋₆ alkyl; said C₁₋₆ alkyl being optionally substituted with OR¹³ or CF₃ wherein R¹³ represents H; R⁶, R⁷, R⁸ and R⁹ each independently represents CH, C—F, C—CF₃ or N; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸ wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ represents H; R² represents CH₂NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸ wherein R16 represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ represents H; R² represents CH₂NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl, particularly cyclopropyl, and R¹¹ and R¹² each represents H.

In one embodiment, each of R¹, R³ and R⁴ represents H; R² represents CH₂NH₂; R⁵ represents C₁₋₆ alkyl; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸ wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ represents H; R² represents CH₂NH₂; R⁵ represents C₁₋₆ alkyl; R⁶, R⁷, R⁸ and R⁹ each represents CH; R¹⁰ represents C3-7 cycloalkyl, particularly cyclopropyl, and R¹¹ and R¹² each represents H.

In one embodiment, each of R¹, R³ and R⁴ represents H; R² represents CH₂NH₂; R⁵ represents C₁₋₆ alkyl; said C₁₋₆ alkyl being substituted with CF₃; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸ wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C₁₋₆ alkyl, and R¹¹ and R¹² each independently represents H or methyl.

In one embodiment, each of R¹, R³ and R⁴ represents H; R² represents CH₂NH₂; R⁵ represents C₁₋₆ alkyl; said C₁₋₆ alkyl being substituted with CF₃; R⁶, R⁷, R⁸ and R⁹ each represents CH; R¹⁰ represents C3-7 cycloalkyl, particularly cyclopropyl, and R¹¹ and R¹² each represents H.

In one embodiment, each of R¹, R³ and R⁴ independently represents H or C1-2 alkyl, particularly methyl; R² represents CH₂NH₂; R⁵ represents C₁₋₆ alkyl; said C₁₋₆ alkyl being optionally substituted with OR¹³ or CF₃; R⁶, R⁷, R⁸ and R⁹ each independently represents CH, C—F, C—Cl or N; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶, halogen or NR¹⁷R¹⁸; and R¹¹ and R¹² each independently represents H or methyl.

In one embodiment, each of R¹, R³ and R⁴ independently represents H or methyl; R² represents CH₂NH₂; R⁵ represents C₁₋₆ alkyl; said C₁₋₆ alkyl being optionally substituted with OR¹³ or CF₃; R⁶, R⁷, R⁸ and R⁹ each represents CH; R¹⁰ represents C3-7 cycloalkyl, particularly cyclopropyl, or C₁₋₆ alkyl; said C₁₋₆ alkyl being optionally substituted with phenyl, C3-7 cycloalkyl, OR¹⁶, SR¹⁶, halogen or NR¹⁷R¹⁸; and R¹¹ and R¹² each independently represents H or methyl.

In one embodiment, each of R¹, R³ and R⁴ independently represents H or methyl; R² represents CH₂NH₂; R⁵ represents C₁₋₆ alkyl; said C₁₋₆ alkyl being optionally substituted with OH or CF₃; R⁶, R⁷, R⁸ and R⁹ each represents CH; R¹⁰ represents C3-7 cycloalkyl, particularly cyclopropyl, or C₁₋₆ alkyl; said C₁₋₆ alkyl being optionally substituted with phenyl, C3-7 cycloalkyl, OH, OCH₃, SCH₃ or NR¹⁷R¹⁸; and R¹¹ and R¹² each independently represents H or methyl.

In one embodiment, each of R¹, R³ and R⁴ represents H; R² represents CH₂NH(CH₂)₃NH₂; R⁵ represents C₁₋₆ alkyl; said C₁₋₆ alkyl being optionally substituted with OH; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸ wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ represents H; R² represents CH₂NH(CH₂)₃NH₂; R⁵ represents C₁₋₆ alkyl; said C₁₋₆ alkyl being optionally substituted with OH; R⁶, R⁷, R⁸ and R⁹ each represents CH; R¹⁰ represents C3-7 cycloalkyl or C₁₋₆ alkyl; said C₁₋₆ alkyl being optionally substituted with OR¹⁶, wherein R¹⁶ represents C₁₋₆ alkyl, particularly methyl, and R¹¹ and R¹² each independently represents H or methyl.

In one embodiment, each of R¹, R³ and R⁴ represents H; R² represents C(═NH)NH₂ or C(═NOH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸, wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C1-6 alkyl, and R¹¹ and R¹² each independently represents H or methyl.

In one embodiment, each of R¹, R³ and R⁴ represents H; R² represents C(═NH)NH₂ or C(═NOH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl, and R¹¹ and R¹² each independently represents H or methyl.

In one embodiment, each of R¹, R³ and R⁴ represents H; R² represents C(═NH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸, wherein R¹⁶ represents C₁₋₆ alkyl, particularly methyl, and the group —NR¹⁷R¹⁸ together represents a 5 to 7 membered azacyclic ring optionally incorporating one further heteroatom selected from O, S and NR²⁰ wherein R²⁰ represents H or C₁₋₆ alkyl, and R¹¹ and R¹² each independently represents H or methyl.

In one embodiment, each of R¹, R³ and R⁴ represents H; R² represents C(═NH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl, and R¹¹ and R¹² each independently represents H or methyl.

In one embodiment, each of R¹, R³ and R⁴ represents H; R² represents C(═NOH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl or C₁₋₆ alkyl; said C₁₋₆ alkyl being optionally substituted with phenyl, C3-7 cycloalkyl, OR¹⁶, SR¹⁶ or NR¹⁷R¹⁸, wherein R¹⁶ represents C₁₋₆ alkyl, particularly methyl, and the group —NR¹⁷R¹⁸ together represents a 5 to 7 membered azacyclic ring optionally incorporating one further is heteroatom selected from O, S and NR²⁰ wherein R²⁰ represents H or C₁₋₆ alkyl, and R¹¹ and R¹² each independently represents H or methyl.

In one embodiment, each of R¹, R³ and R⁴ represents H; R² represents C(═NOH)NH₂; R⁵ represents C₁₋₆ alkyl; said C₁₋₆ alkyl being optionally substituted with CF₃; R⁶, R⁷, R⁸ and R⁹ each represents CH; R¹⁰ represents C3-7 cycloalkyl, and R¹¹ and R¹² each independently represents H or methyl.

Examples of compounds of the invention include:

3-methyl-1-[(1-isopentylbenzimidazol-2-yl)methyl]-4H-quinazolin-2-one;

3-isopentyl-1-[(1-isopentylbenzimidazol-2-yl)methyl]-4H-quinazolin-2-one;

3-cyclopropyl-1-[(1-isopentylbenzimidazol-2-yl)methyl]-4-methyl-4H-quinazolin-2-one;

3-cyclopropyl-1-[(1-isopentylbenzimidazol-2-yl)methyl]-4,4-dimethyl-quinazolin-2-one;

1-[[5-(aminomethyl)-1-isopentyl-benzimidazol-2-yl]methyl]-3-methyl-4H-quinazolin-2-one;

1-[[5-(aminomethyl)-1-isopentyl-benzimidazol-2-yl]methyl]-3-propyl-4H-quinazolin-2-one;

1-[[5-(aminomethyl)-1-isopentyl-benzimidazol-2-yl]methyl]-3-cyclopropyl-4H-quinazolin-2-one;

1-[[5-(aminomethyl)-1-isopentyl-benzimidazol-2-yl]methyl]-3-tert-butyl-4H-quinazolin-2-one;

1-[[5-(aminomethyl)-1-isopentyl-benzimidazol-2-yl]methyl]-3-cyclopentyl-4H-quinazolin-2-one;

1-[[5-(aminomethyl)-1-isopentyl-benzimidazol-2-yl]methyl]-3-benzyl-4H-quinazolin-2-one;

1-[[5-(aminomethyl)-1-isopentyl-benzimidazol-2-yl]methyl]-3-phenethyl-4H-quinazolin-2-one;

1-[[5-(aminomethyl)-1-isopentyl-benzimidazol-2-yl]methyl]-3-cyclopropyl-4H-pyrido[2,3-d]pyrimidin-2-one;

1-[[5-(aminomethyl)-1-isopentyl-benzimidazol-2-yl]methyl]-3-(2-methoxyethyl)-4H-quinazolin-2-one;

1-[[5-(aminomethyl)-1-isopentyl-benzimidazol-2-yl]methyl]-3-isopentyl-4H-quinazolin-2-one;

1-[[5-(aminomethyl)-1-isopentyl-benzimidazol-2-yl]methyl]-3-isobutyl-4H-quinazolin-2-one;

1-[[5-(aminomethyl)-1-isopentyl-benzimidazol-2-yl]methyl]-3-(cyclopropylmethyl)-4H-quinazolin-2-one;

1-[[5-(aminomethyl)-1-isopentyl-benzimidazol-2-yl]methyl]-3-(3-pyrrolidin-1-ylpropyl)-4H-quinazolin-2-one;

1-[[5-(aminomethyl)-1-isopentyl-benzimidazol-2-yl]methyl]-3-(2-methylsulfanylethyl)-4H-quinazolin-2-one;

1-[[5-(aminomethyl)-1-isopentyl-benzimidazol-2-yl]methyl]-3-(cyclohexylmethyl)-4H-quinazolin-2-one;

1-[[5-(aminomethyl)-1-isopentyl-benzimidazol-2-yl]methyl]-3-cyclopropyl-4-methyl-4H-quinazolin-2-one;

1-[[5-(aminomethyl)-1-isopentyl-benzimidazol-2-yl]methyl]-3-cyclopropyl-4,4-dimethyl-quinazolin-2-one;

1-[[5-(aminomethyl)-1-isopentyl-benzimidazol-2-yl]methyl]-3-cyclopropyl-5-(trifluoromethyl)-4H-quinazolin-2-one;

1-[[5-(aminomethyl)-1-isopentyl-benzimidazol-2-yl]methyl]-3-cyclopropyl-5-fluoro-4H-quinazolin-2-one;

1-[[5-(aminomethyl)-1-(4-hydroxybutyl)benzimidazol-2-yl]methyl]-3-methyl-4H-quinazolin-2-one;

1-[[5-(aminomethyl)-1-(4-hydroxybutyl)benzimidazol-2-yl]methyl]-3-cyclopropyl-4H-quinazolin-2-one;

1-[[5-(aminomethyl)-1-(4-hydroxybutyl)benzimidazol-2-yl]methyl]-3-(2-methoxyethyl)-4H-quinazolin-2-one;

1-[[5-(aminomethyl)-1-(4-hydroxybutyl)benzimidazol-2-yl]methyl]-3-(cyclohexylmethyl)-4H-quinazolin-2-one;

1-[[5-(aminomethyl)-1-(4,4,4-trifluorobutyl)benzimidazol-2-yl]methyl]-3-cyclopropyl-4H-quinazolin-2-one;

1-[[5-(aminomethyl)-1-(4,4,4-trifluorobutyl)benzimidazol-2-yl]methyl]-3-cyclopropyl-4-methyl-4H-quinazolin-2-one;

1-[[5-(aminomethyl)-1-(4,4,4-trifluorobutyl)benzimidazol-2-yl]methyl]-3-cyclopropyl-4,4-dimethyl-quinazolin-2-one;

1-[[5-[(3-aminopropylamino)methyl]-1-isopentyl-benzimidazol-2-yl]methyl]-3-methyl-4H-quinazolin-2-one;

1-[[5-[(3-aminopropylamino)methyl]-1-isopentyl-benzimidazol-2-yl]methyl]-3-cyclopropyl-4H-quinazolin-2-one;

1-[[5-[(3-aminopropylamino)methyl]-1-isopentyl-benzimidazol-2-yl]methyl]-3-(2-methoxyethyl)-4H-quinazolin-2-one;

1-[[5-[(3-aminopropylamino)methyl]-1-(4-hydroxybutyl)benzimidazol-2-yl]methyl]-3-methyl-4H-quinazolin-2-one;

2-[(3-cyclopropyl-2-oxo-4H-quinazolin-1-yl)methyl]-1-isopentyl-benzimidazole-5-carboxamidine;

2-[(3-cyclopropyl-4-methyl-2-oxo-4H-quinazolin-l-yl)methyl]-1-isopentyl-benzimidazole-5-carboxamidine;

2-[(3-cyclopropyl-4,4-dimethyl-2-oxo-quinazolin-1-yl)methyl]-1-isopentyl-benzimidazole-5-carboxamidine;

2-[(3-cyclopropyl-2-oxo-4H-quinazolin-1-yl)methyl]-N′-hydroxy-1-isopentyl-benzimidazole-5-carboxamidine;

2-[(3-cyclopropyl-4-methyl-2-oxo-4H-quinazolin-1-yl)methyl]-N′-hydroxy-1-isopentyl-benzimidazole-5-carboxamidine;

2-[(3-cyclopropyl-4,4-dimethyl-2-oxo-quinazolin-1-yl)methyl]-N′-hydroxy-1-isopentyl-benzimidazole-5-carboxamidine;

2-[(3-cyclopropyl-2-oxo-4H-quinazolin-1-yl)methyl]-1-(4,4,4-trifluorobutyl)benzimidazole-5-carboxamidine;

2-[(3-cyclopropyl-4-methyl-2-oxo-4H-quinazolin-1-yl)methyl]-1-(4,4,4-trifluorobutyl)benzimidazole-5-carboxamidine;

2-[(3-cyclopropyl-4,4-dimethyl-2-oxo-quinazolin-1-yl)methyl]-1-(4,4,4-trifluorobutyl)benzimidazole-5-carboxamidine;

2-[(3-cyclopropyl-4-methyl-2-oxo-4H-quinazolin-1-yl)methyl]-N′-hydroxy-1-(4,4,4-trifluorobutyl)benzimidazole-5-carboxamidine;

2-[(3-cyclopropyl-4,4-dimethyl-2-oxo-quinazolin-1-yl)methyl]-N′-hydroxy-1-(4,4,4-trifluorobutyl)benzimidazole-5-carboxamidine;

1-[[5-(aminomethyl)-1-isopentyl-6-methyl-benzimidazol-2-yl]methyl]-3-cyclopropyl-4H-quinazolin-2-one;

and pharmaceutically acceptable salts thereof.

In another embodiment of the invention there is therefore provided 1-[[5-(aminomethyl)-1-isopentyl-benzimidazol-2-yl]methyl]-3-cyclopropyl-4H-quinazolin-2-one, or a pharmaceutically acceptable salt thereof. In another embodiment of the invention, there is therefore provided 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

Compounds of formula (I) are capable of existing in stereoisomeric forms. It will be understood that the invention encompasses the use of all geometric and optical isomers of the compounds of formula (I) and mixtures thereof, including racemates. The use of tautomers and mixtures thereof also form an aspect of the present invention. Enantiomerically pure forms are particularly desired.

Compounds of formula (I) may exist in crystalline form and exhibit polymorphism. It will be understood that the invention encompasses the use of all polymorphic forms of the compounds of formula (I). Thus, in one embodiment of the present invention, there is provided a compound of formula (I), or a pharmaceutically acceptable salt thereof, in crystalline form.

Compounds of formula (I) may exist in unsolvated forms as well as solvated forms, such as, for example, hydrated forms. It will be understood that the invention encompasses all unsolvated and solvated forms of the compounds of formula (I). Solvated forms of the compounds of formula (I) may be used as intermediates in the preparation of further compounds of formula (I).

In one embodiment of the invention, there is provided an amphorous form of a compound of formula (I), or a pharmaceutically acceptable salt thereof. In another embodiment of the invention, there is provided an amphorous form of 1-[[5-(aminomethyl)-1-isopentyl-benzimidazol-2-yl]methyl]-3-cyclopropyl-4H-quinazolin-2-one, or a pharmaceutically acceptable salt thereof. In one embodiment of the invention, there is provided an amphorous form of 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

As stated hereinbefore, certain compounds of formula (I) may exist in crystalline form and exhibit polymorphism. According to the invention there is therefore provided a crystalline form of a compound of formula (I), or a pharmaceutically acceptable salt thereof. In another embodiment of the invention there is therefore provided a crystalline form of 1-[[5-(aminomethyl)-1-isopentyl-benzimidazol-2-yl]methyl]-3-cyclopropyl-4H-quinazolin-2-one, or a pharmaceutically acceptable salt thereof. In another embodiment of the invention, there is therefore provided a crystalline form of 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

As stated hereinbefore, certain compounds of formula (I) may exist in a solvated form. According to the invention there is therefore provided a solvated form of a compound of formula (I), or a pharmaceutically acceptable salt thereof. In another embodiment of the invention, there is provided a solvated form of 1-[[5-(aminomethyl)-1-isopentyl-benzimidazol-2-yl]methyl]-3-cyclopropyl-4H-quinazolin-2-one, or a pharmaceutically acceptable salt thereof. In another embodiment of the invention, there is provided a solvated form of 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

In one embodiment of the invention, there is provided a crystalline form of 1-[[5-(aminomethyl)-1-isopentyl-benzimidazol-2-yl]methyl]-3-cyclopropyl-4H-quinazolin-2-one monohydrate, which has an X-ray powder diffraction pattern with at least one specific peak at a 2-theta value of about 8.2, 9.7, 16.3, 8.4, 24.6, 19.5, 22.7, 21.9, 23.3 or 14.6° when measured using CuKa radiation, more particularly wherein said values may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one monohydrate, which has an X-ray powder diffraction pattern with specific peaks at 2-theta values of about 8.2, 9.7, 16.3, 8.4, 24.6, 19.5, 22.7, 21.9, 23.3 and 14.6° when measured using CuKa radiation, more particularly wherein said values may be plus or minus 0.5° 2-theta.

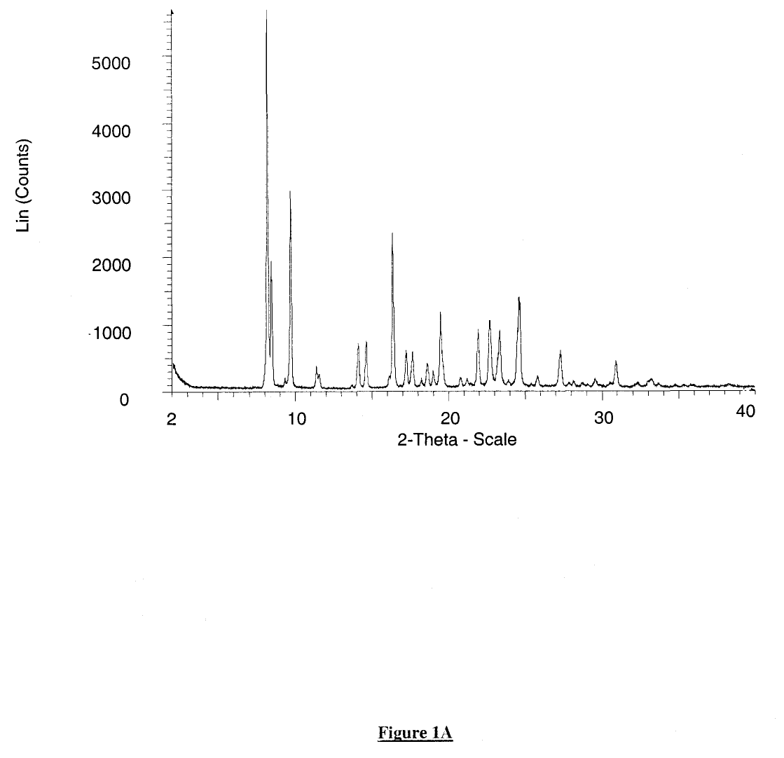

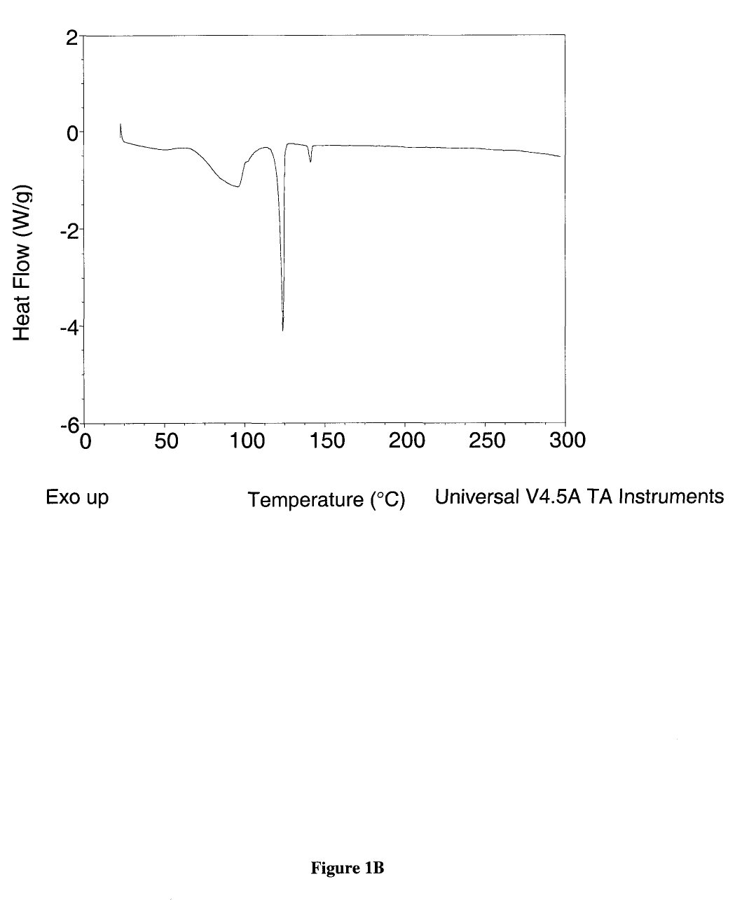

In one embodiment of the invention, there is provided crystalline form of 1-[[5-(aminomethyl)-1-isopentyl-benzimidazol-2-yl]methyl]-3-cyclopropyl-4H-quinazolin-2-one monohydrate, which has an X-ray powder diffraction pattern substantially the same as the X-ray powder diffraction pattern shown in FIG. 1A when measured using CuKa radiation.

In one embodiment of the invention there is provided a crystalline form of 1-[[5-(aminomethyl)-1-isopentyl-benzimidazol-2-yl]methyl]-3-cyclopropyl-4H-quinazolin-2-one dihydrate, which has an X-ray powder diffraction pattern with at least one specific peak at a 2-theta value of about 8.9, 8.5, 21.4, 8.2, 17.7, 21.7, 12.6, 22.5, 17.2 or 12.3° when measured using CuKa radiation, more particularly wherein said value may be plus or minus 0.5° 2-theta.

In one embodiment of the invention there is provided a crystalline form of 1-[[5-(aminomethyl)-1-isopentyl-benzimidazol-2-yl]methyl]-3-cyclopropyl-4H-quinazolin-2-one dihydrate, which has an X-ray powder diffraction pattern with specific peaks at 2-theta values of about 8.9, 8.5, 21.4, 8.2, 17.7, 21.7, 12.6, 22.5, 17.2 and 12.3° when measured using CuKa radiation, more particularly wherein said value may be plus or minus 0.5° 2-theta.

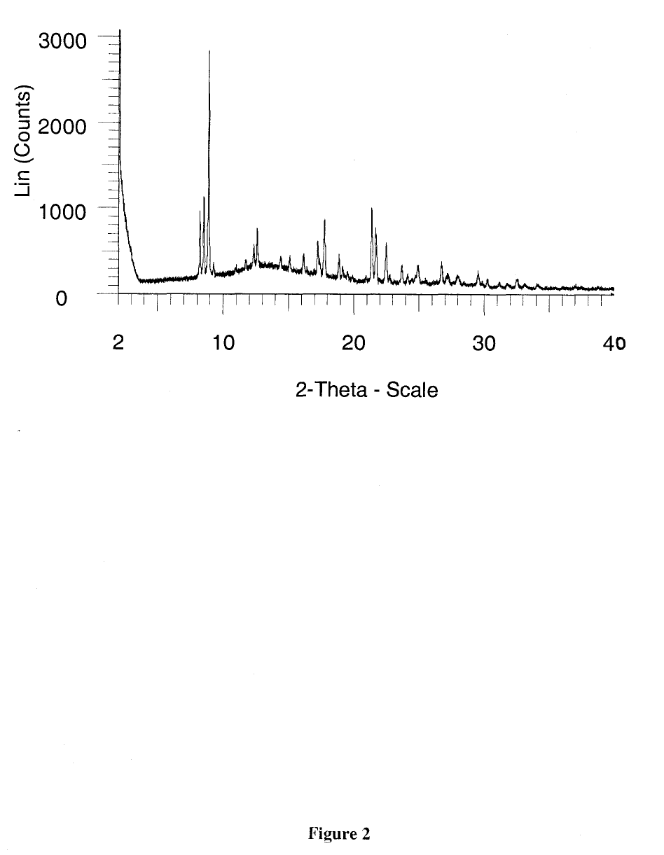

is In one embodiment of the invention there is provided crystalline form of 1-[[5-(aminomethyl)-1-isopentyl-benzimidazol-2-yl]methyl]-3-cyclopropyl-4H-quinazolin-2-one dihydrate, which has an X-ray powder diffraction pattern substantially the same as the X-ray powder diffraction pattern shown in FIG. 2 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form B, which has an X-ray powder diffraction pattern with at least one specific peak at a 2-theta value of about 19.9, 21.2, 20.3, 8.7, 10.2, 15.5, 11.8, 22.0, 13.0 or 13.8° when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form B, which has an X-ray powder diffraction pattern with specific peaks at 2-theta values of about 19.9, 21.2, 20.3, 8.7, 10.2, 15.5, 11.8, 22.0, 13.0 and 13.8° when measured using CuKa radiation, more particularly wherein said value may be plus or minus 0.5° 2-theta.

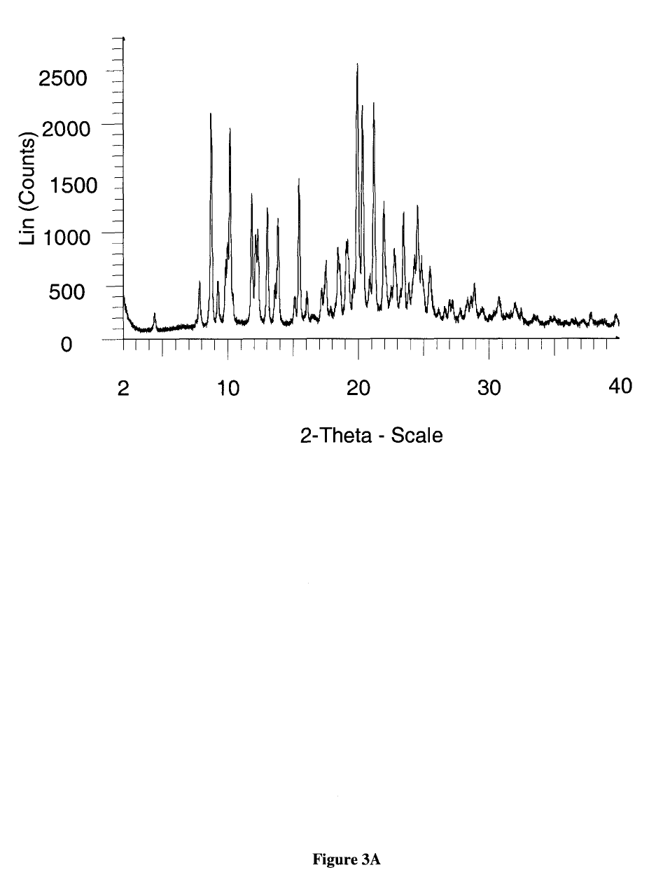

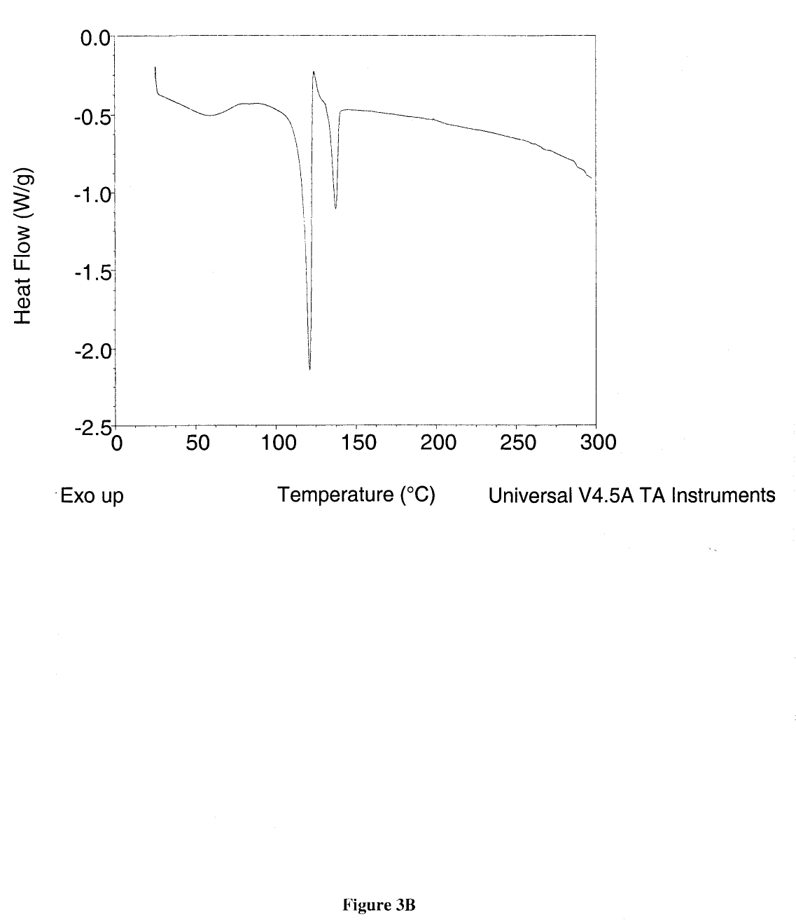

In one embodiment of the invention, there is provided crystalline form of 1-[[5-(aminomethyl)-1-isopentyl-benzimidazol-2-yl]methyl]-3-cyclopropyl-4H-quinazolin-2-one, Form B, which has an X-ray powder diffraction pattern substantially the same as the X-ray powder diffraction pattern shown in FIG. 3A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form C, which has an X-ray powder diffraction pattern with at least one specific peak at a 2-theta value of about 9.8, 6.9, 20.5, 14.3, 22.9, 24.4, 20.9, 24.7, 9.4 or 11.2° when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form C, which has an X-ray powder diffraction pattern with specific peaks at 2-theta values of about 9.8, 6.9, 20.5, 14.3, 22.9, 24.4, 20.9, 24.7, 9.4 and 11.2° when measured using CuKa radiation, more particularly wherein said value may be plus or minus 0.5° 2-theta.

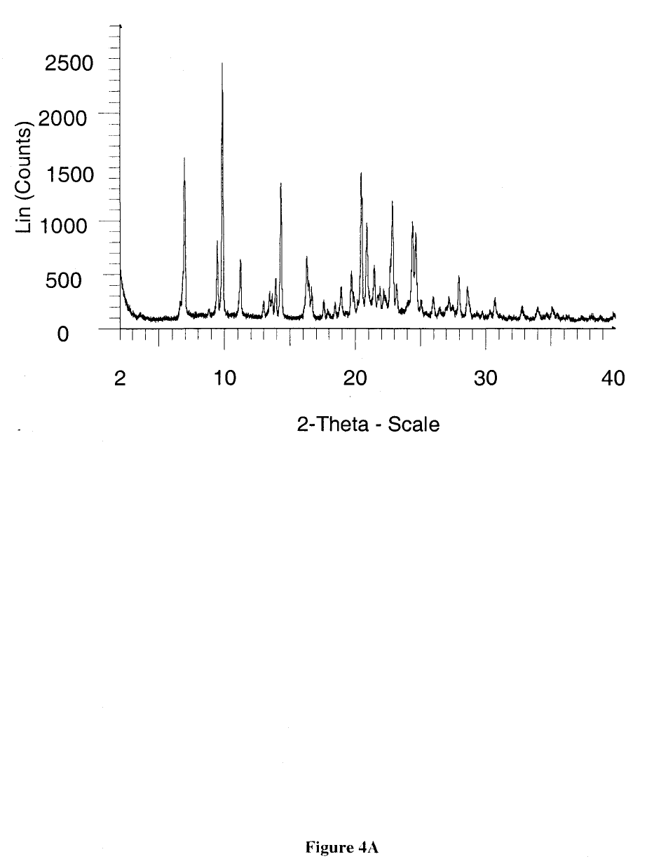

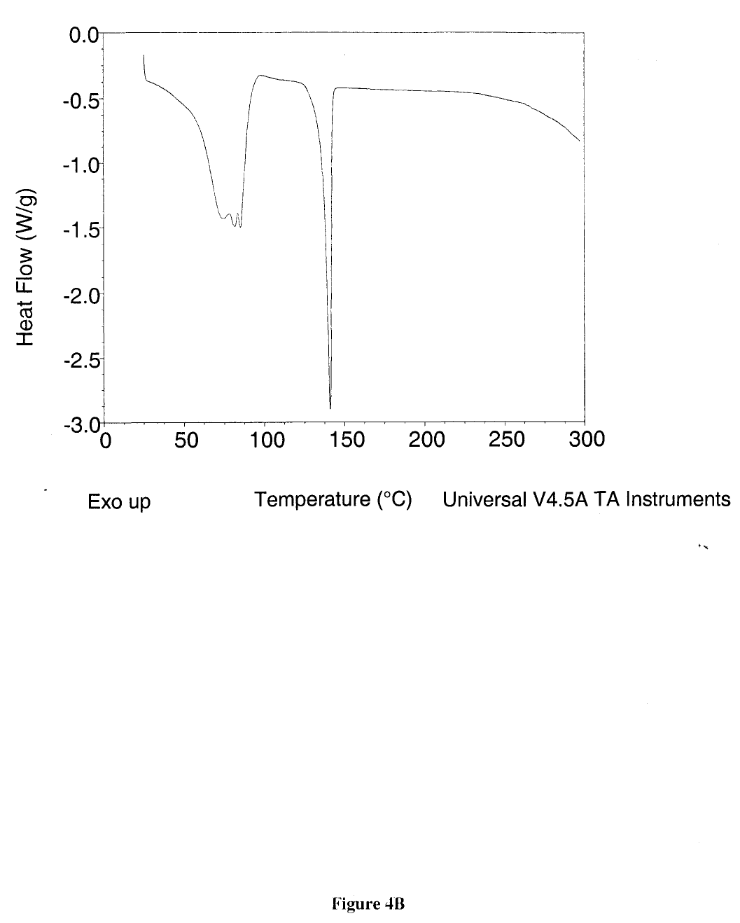

In one embodiment of the invention, there is provided crystalline form of 1-[[5-(aminomethyl)-1-isopentyl-benzimidazol-2-yl]methyl]-3-cyclopropyl-4H-quinazolin-2-one, Form C, which has an X-ray powder diffraction pattern substantially the same as the X-ray powder diffraction pattern shown in FIG. 4A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form D, which has an X-ray powder diffraction pattern with at least one specific peak at a 2-theta value of about 10.4, 12.2, 18.2, 17.1, 19.6, 7.5, 19.3, 22.5, 20.2 or 8.7° when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form D, which has an X-ray powder diffraction pattern with specific peaks at 2-theta values of about 10.4, 12.2, 18.2, 17.1, 19.6, 7.5, 19.3, 22.5, 20.2 and 8.7° when measured using CuKa radiation, more particularly wherein said value may be plus or minus 0.5° 2-theta.

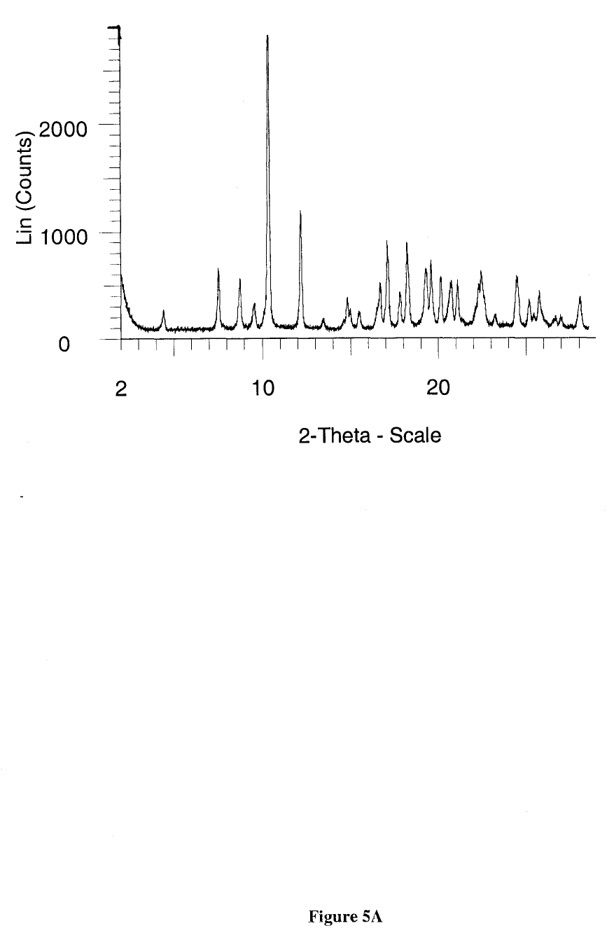

In one embodiment of the invention, there is provided crystalline form of 1-[[5-(aminomethyl)-1-isopentyl-benzimidazol-2-yl]methyl]-3-cyclopropyl-4H-quinazolin-2-one, Form D, which has an X-ray powder diffraction pattern substantially the same as the X-ray powder diffraction pattern shown in FIG. 5A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form E, which has an X-ray powder diffraction pattern with at least one specific peak at a 2-theta value of about 8.7, 21.6, 17.2, 8.2, 12.8, 18.7, 12.3, 19.7, 24.8 or 19.3° when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form E, which has an X-ray powder diffraction pattern with specific peaks at 2-theta values of about 8.7, 21.6, 17.2, 8.2, 12.8, 18.7, 12.3, 19.7, 24.8 and 19.3° when measured using CuKa radiation, more particularly wherein said value may be plus or minus 0.5° 2-theta.

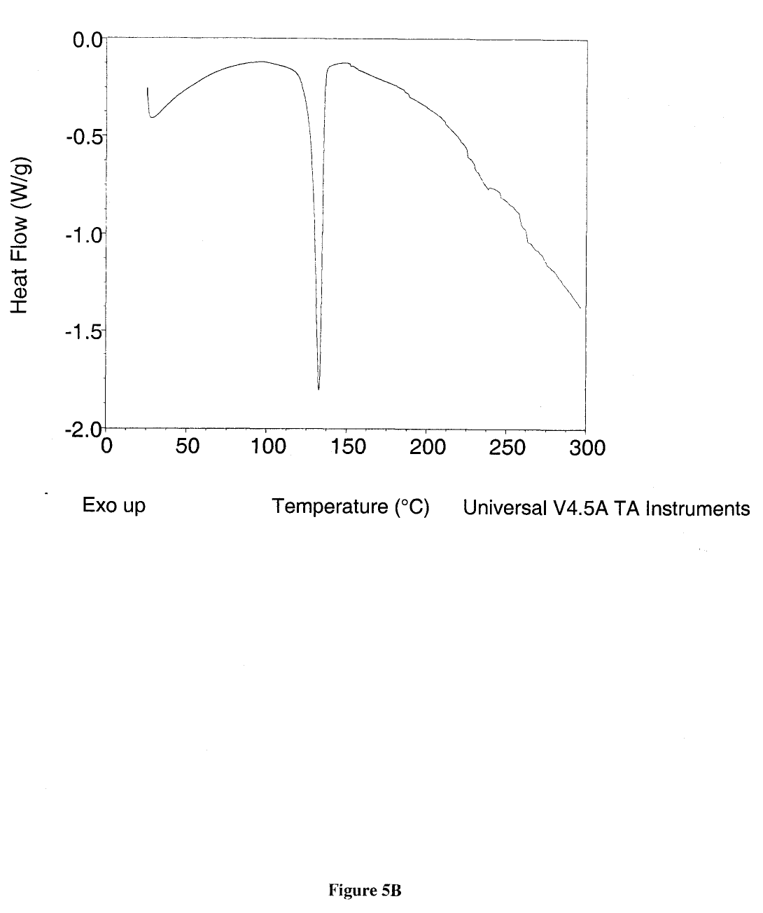

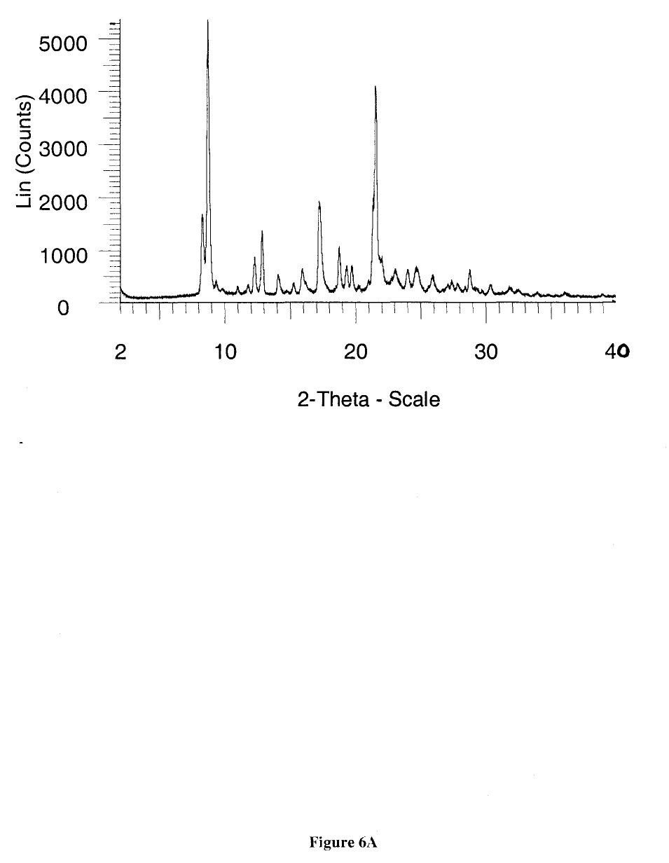

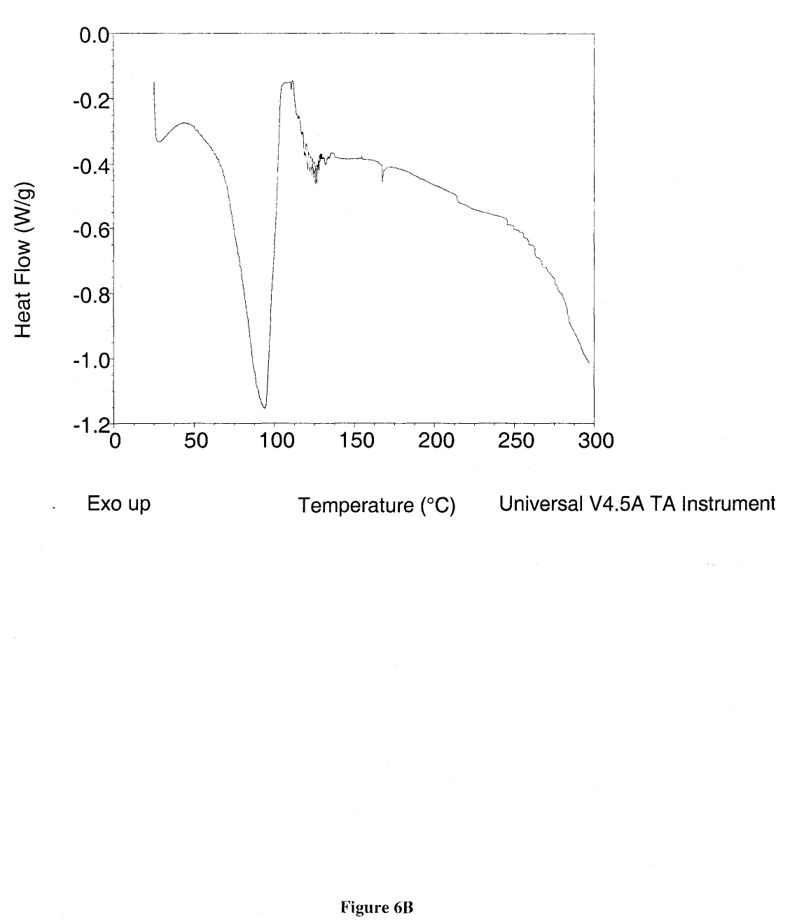

In one embodiment of the invention, there is provided crystalline form of 1-[[5-(aminomethyl)-1-isopentyl-benzimidazol-2-yl]methyl]-3-cyclopropyl-4H-quinazolin-2-one, Form E, which has an X-ray powder diffraction pattern substantially the same as the X-ray powder diffraction pattern shown in FIG. 6A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form F, which has an X-ray powder diffraction pattern with at least one specific peak at a 2-theta value of about 8.8, 8.4, 8.2, 4.1, 21.4, 22.5 or 21.8° when measured using CuKa radiation, more particularly wherein said values may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form F, which has an X-ray powder diffraction pattern with specific peaks at 2-theta values of about 8.8, 8.4, 8.2, 4.1, 21.4, 22.5 and 21.8° when measured using CuKa radiation, more particularly wherein said values may be plus or minus 0.5° 2-theta.

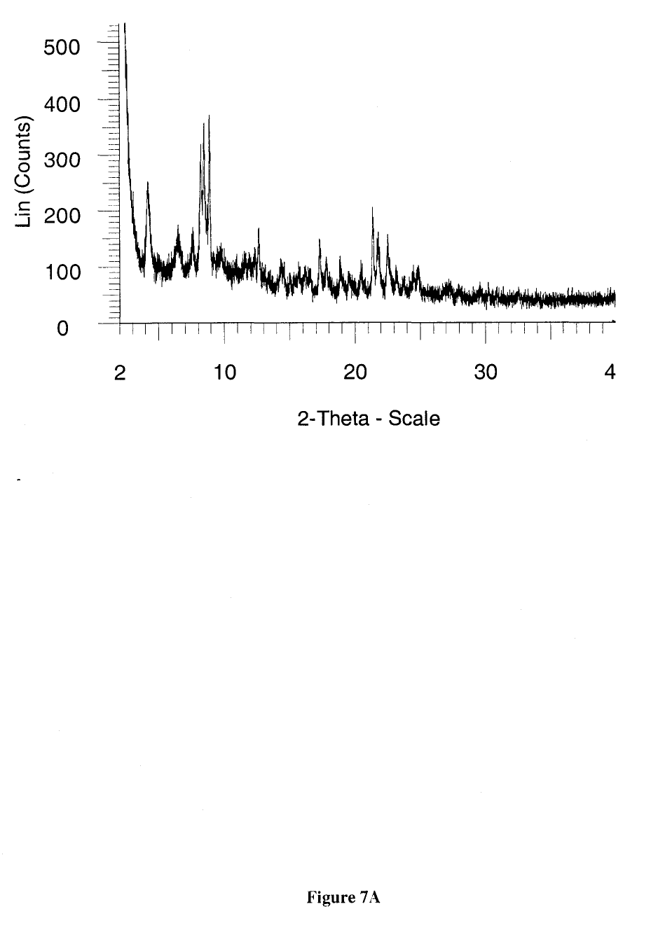

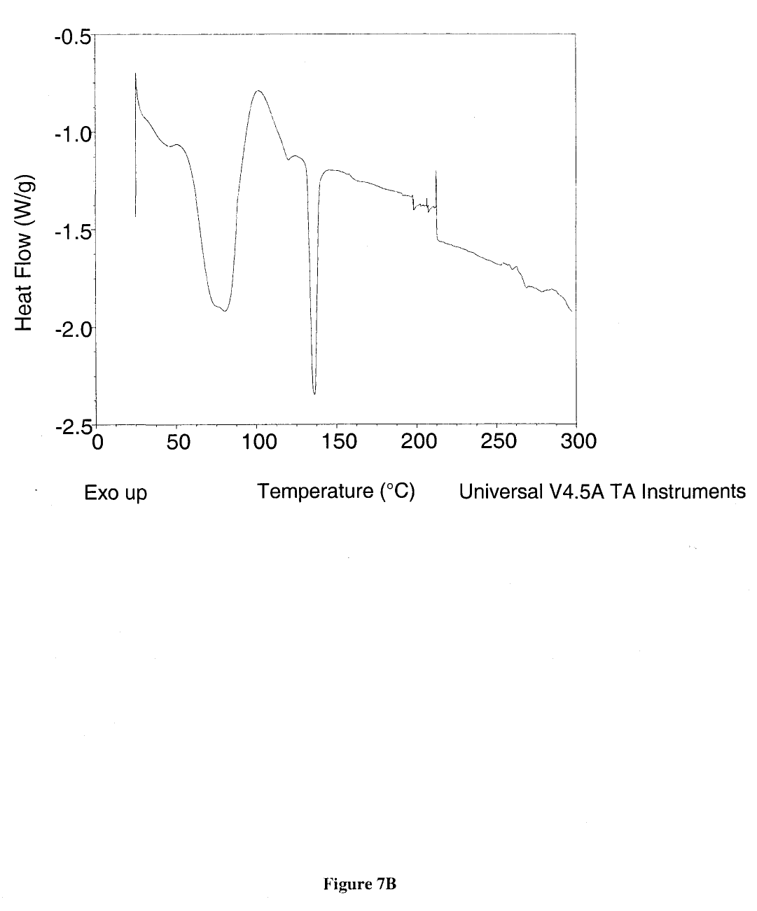

In one embodiment of the invention, there is provided crystalline form of 1-[[5-(aminomethyl)-1-isopentyl-benzimidazol-2-yl]methyl]-3-cyclopropyl-4H-quinazolin-2-one, Form F, which has an X-ray powder diffraction pattern substantially the same as the X-ray powder diffraction pattern shown in FIG. 7A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form G, which has an X-ray powder diffraction pattern with at least one specific peak at a 2-theta value of about 10.4, 12.2, 19.6, 18.2, 17.2, 21.9, 24.6, 9.7, 7.5 or 25.7° when measured using CuKa radiation, more particularly wherein said values may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form G, which has an X-ray powder diffraction pattern with specific peaks at 2-theta values of about 10.4, 12.2, 19.6, 18.2, 17.2, 21.9, 24.6, 9.7, 7.5 and 25.7° when measured using CuKa radiation, more particularly wherein said values may be plus or minus 0.5° 2-theta.

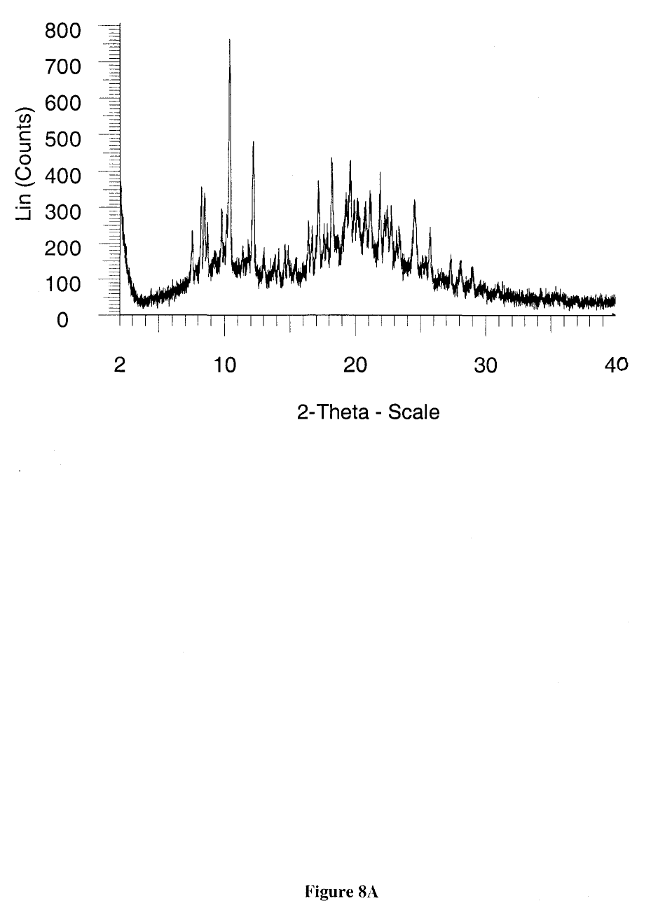

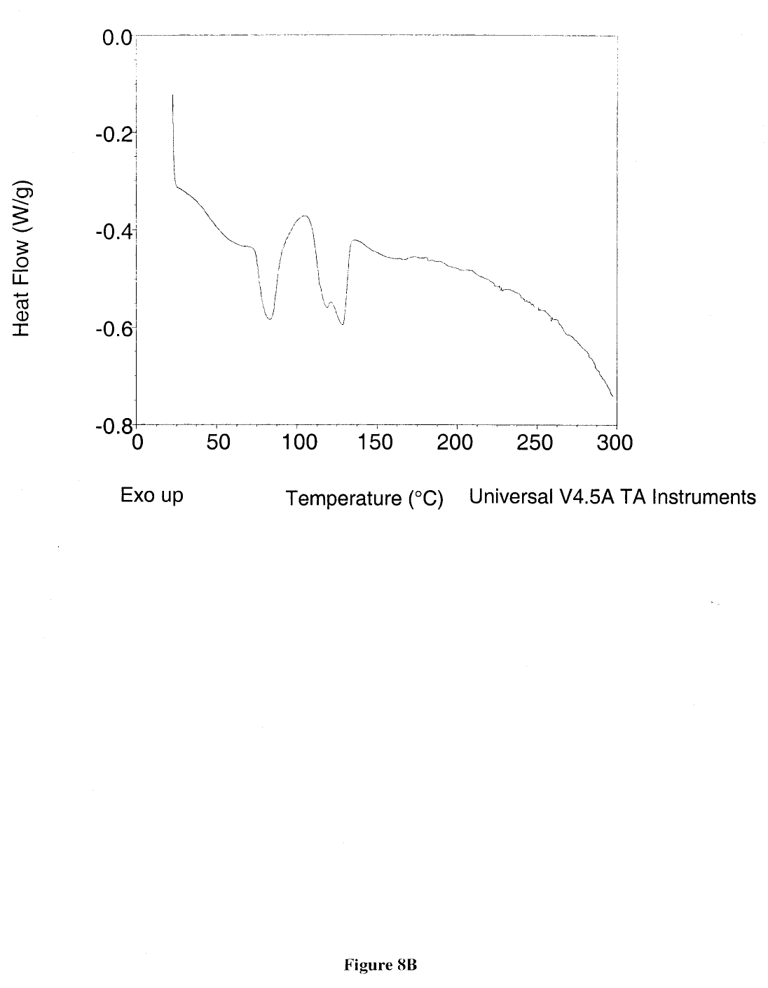

In one embodiment of the invention, there is provided crystalline form of 1-[[5-(aminomethyl)-1-isopentyl-benzimidazol-2-yl]methyl]-3-cyclopropyl-4H-quinazolin-2-one, Form G, which has an X-ray powder diffraction pattern substantially the same as the X-ray powder diffraction pattern shown in FIG. 8A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form H, which has an X-ray powder diffraction pattern with at least one specific peak at a 2-theta value of about 19.9, 9.8, 21.9, 13.5, 25.6, 13.9, 17.2, 10.2, 22.9 or 13.1° when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one, Form H, which has an X-ray powder diffraction pattern with specific peaks at 2-theta values of about 19.9, 9.8, 21.9, 13.5, 25.6, 13.9, 17.2, 10.2, 22.9 and 13.1° when measured using CuKa radiation, more particularly wherein said value may be plus or minus 0.5° 2-theta.

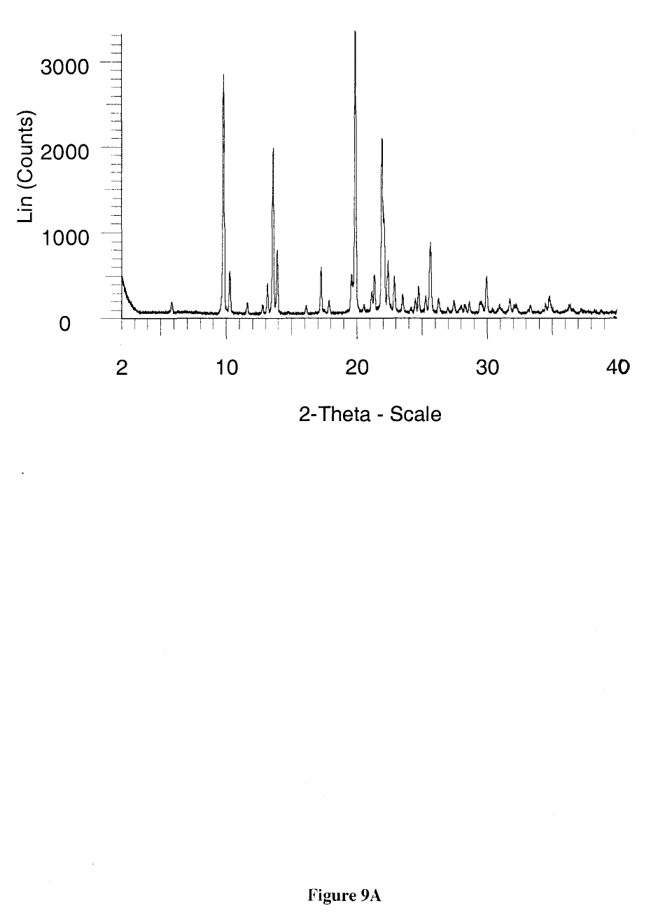

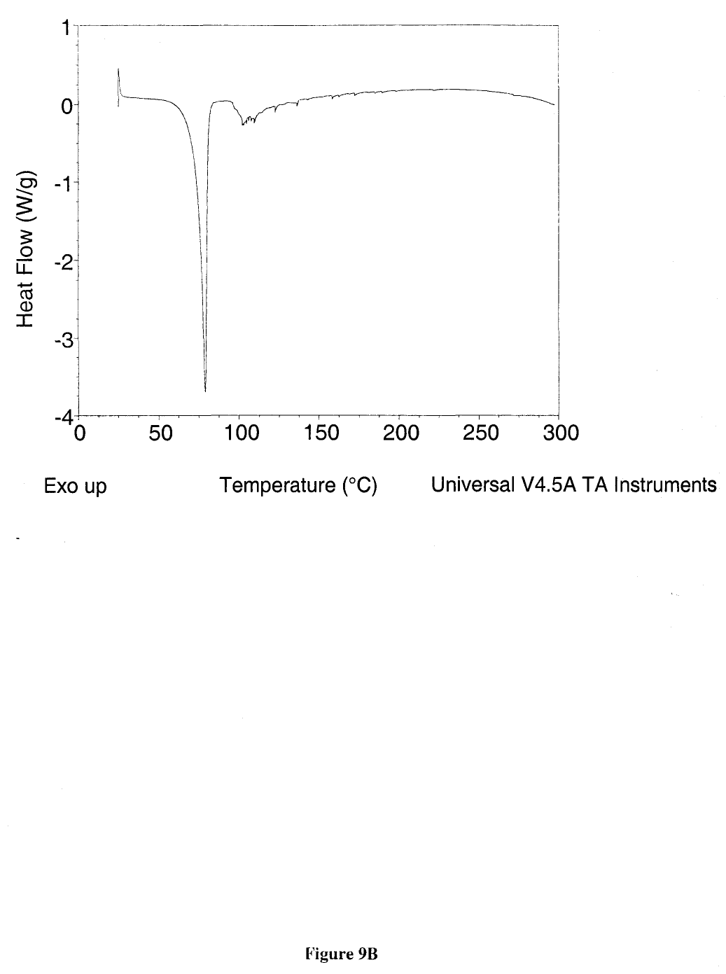

In one embodiment of the invention, there is provided crystalline form of 1-[[5-(aminomethyl)-1-isopentyl-benzimidazol-2-yl]methyl]-3-cyclopropyl-4H-quinazolin-2-one, Form H, which has an X-ray powder diffraction pattern substantially the same as the X-ray powder diffraction pattern shown in FIG. 9A when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form A, which has an X-ray powder diffraction pattern with at least one specific peak at a 2-theta value of about 22.4, 12.6, 27.7, 18.1, 20.7, 6.9, 18.8, 9.4, 10.9 or 13.8 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form A, which has an X-ray powder diffraction pattern with specific peaks at 2-theta values of about 22.4, 12.6, 27.7, 18.1, 20.7, 6.9, 18.8, 9.4, 10.9 and 13.8 when measured using CuKa radiation, more particularly wherein said value may be plus or minus 0.5° 2-theta.

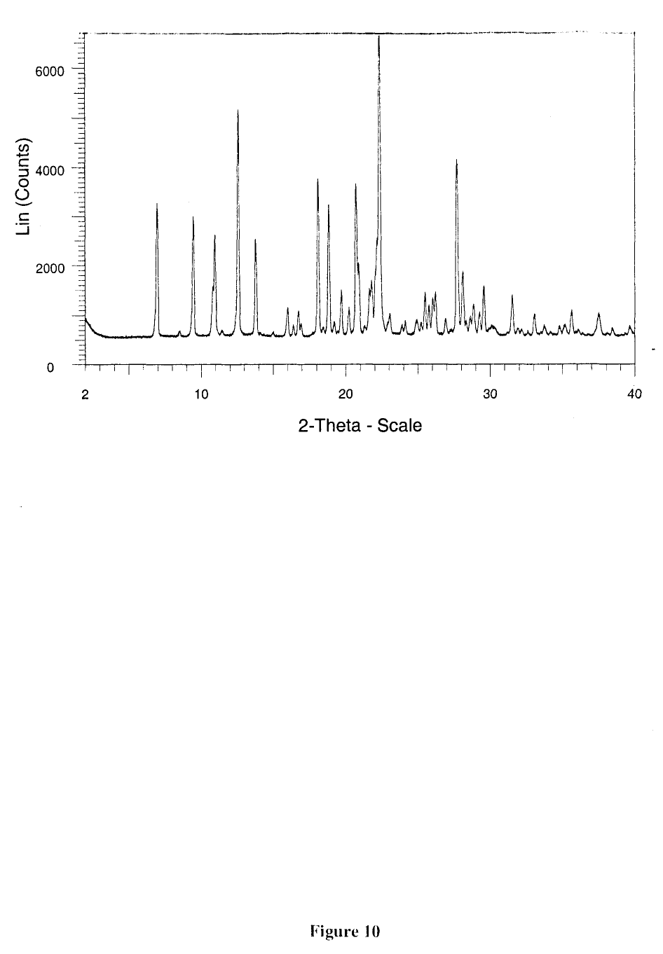

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form A, which has an X-ray powder diffraction pattern substantially as shown in FIG. 10 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form B, which has an X-ray powder diffraction pattern with at least one specific peak at a 2-theta value of about 8.2, 9.4, 14.7, 19.8, 4.1, 7.3, 14.1, 18.8, 22.1 or 12.5 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form B, which has an X-ray powder diffraction pattern with specific peaks at 2-theta values of about 8.2, 9.4, 14.7, 19.8, 4.1, 7.3, 14.1, 18.8, 22.1 and 12.5 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one acetate salt, Form B, which has an X-ray powder diffraction pattern substantially as shown in FIG. 11 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form A, which has an X-ray powder diffraction pattern with at least one specific peak at a 2-theta value of about 18.2, 22.6, 12.7, 21.6, 9.4, 22.0, 18.7, 20.6, 9.7 or 11.8 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form A, which has an X-ray powder diffraction pattern with specific peaks at 2-theta values of about 18.2, 22.6, 12.7, 21.6, 9.4, 22.0, 18.7, 20.6, 9.7 and 11.8 when measured using CuKa radiation, more particularly wherein said value may be plus or minus 0.5° 2-theta.

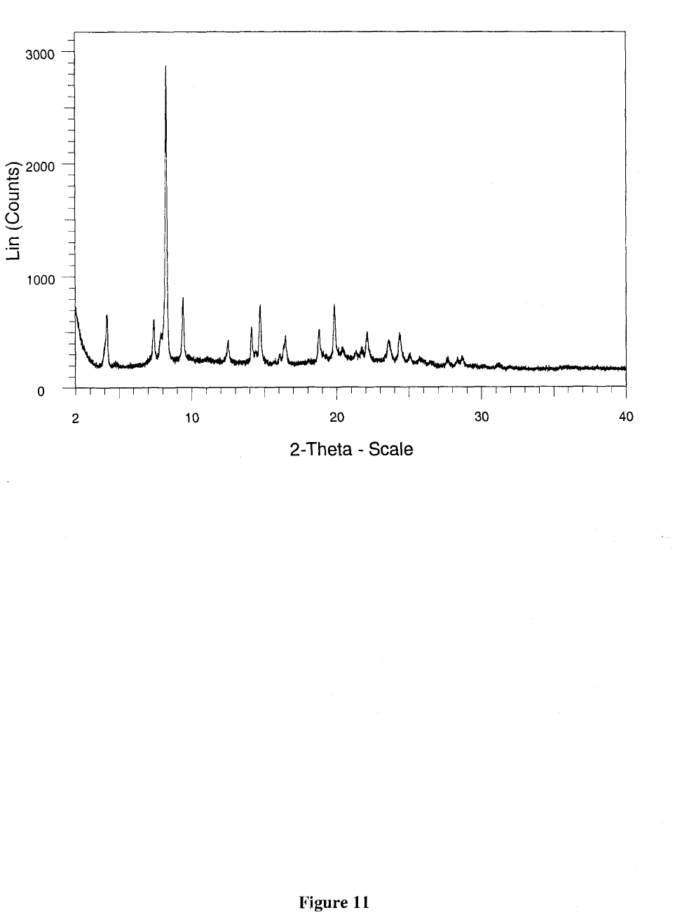

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form A, which has an X-ray powder diffraction pattern substantially as shown in FIG. 12 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form B, which has an X-ray powder diffraction pattern with at least one specific peak at a 2-theta value of about 9.1, 22.0, 11.8, 22.4, 10.7, 23.5, 24.1 or 6.0 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form B, which has an X-ray powder diffraction pattern with specific peaks at 2-theta values of about 9.1, 22.0, 11.8, 22.4, 10.7, 23.5, 24.1 and 6.0 when measured using CuKa radiation, more particularly wherein said value may be plus or minus 0.5° 2-theta.

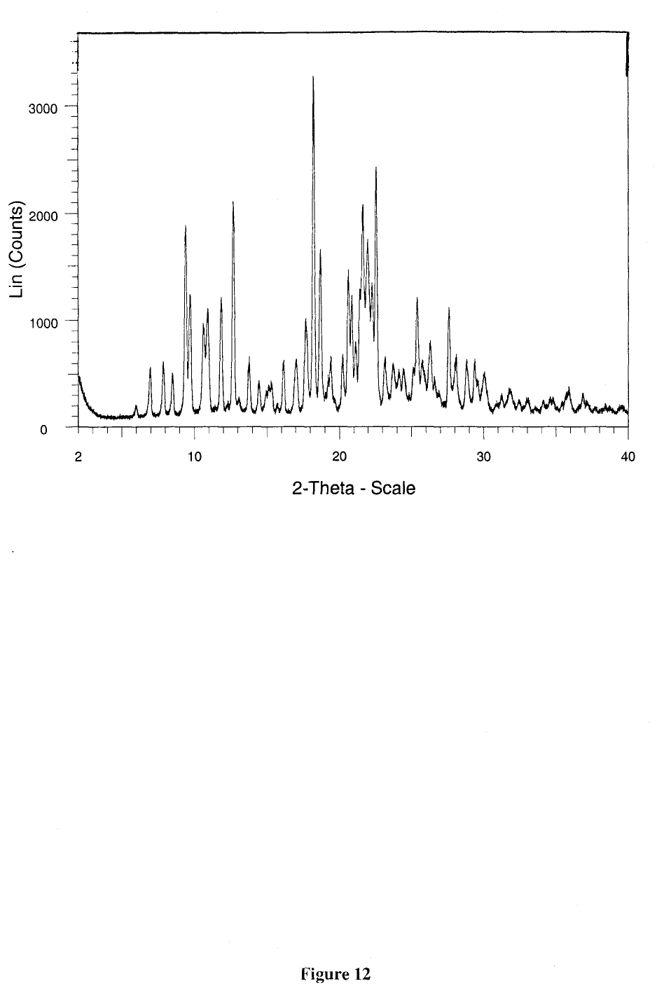

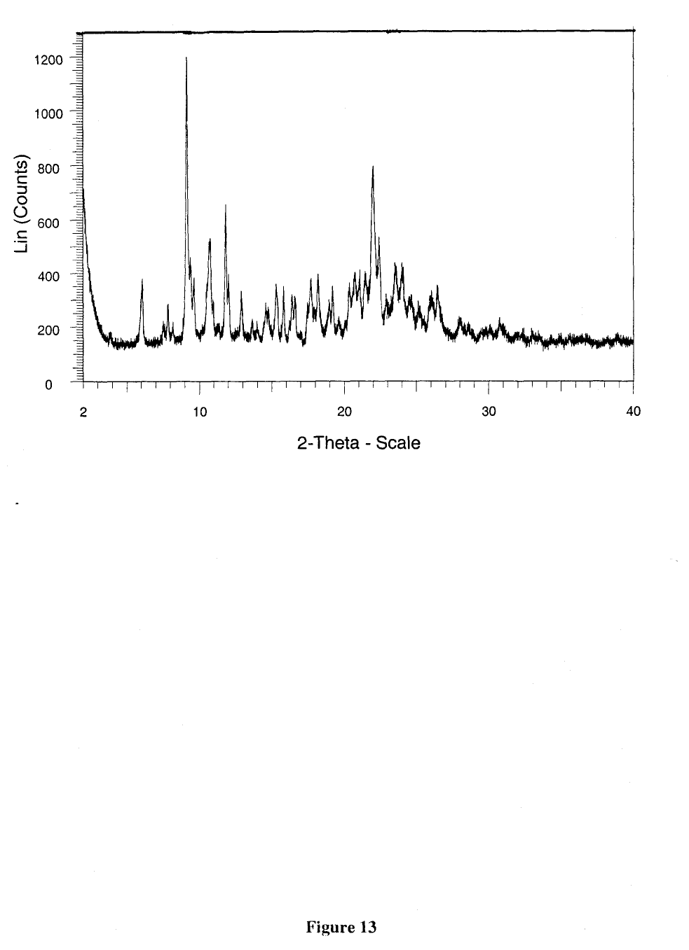

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form B, which has an X-ray powder diffraction pattern substantially as shown in FIG. 13 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form C, which has an X-ray powder diffraction pattern with at least one specific peak at a 2-theta value of about 23.3, 9.4, 10.3, 22.6, 18.3, 21.6, 23.0, 25.4, 18.7 or 12.7 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form C, which has an X-ray powder diffraction pattern with specific peaks at 2-theta values of about 23.3, 9.4, 10.3, 22.6, 18.3, 21.6, 23.0, 25.4, 18.7 and 12.7 when measured using CuKa radiation, more particularly wherein said value may be plus or minus 0.5° 2-theta.

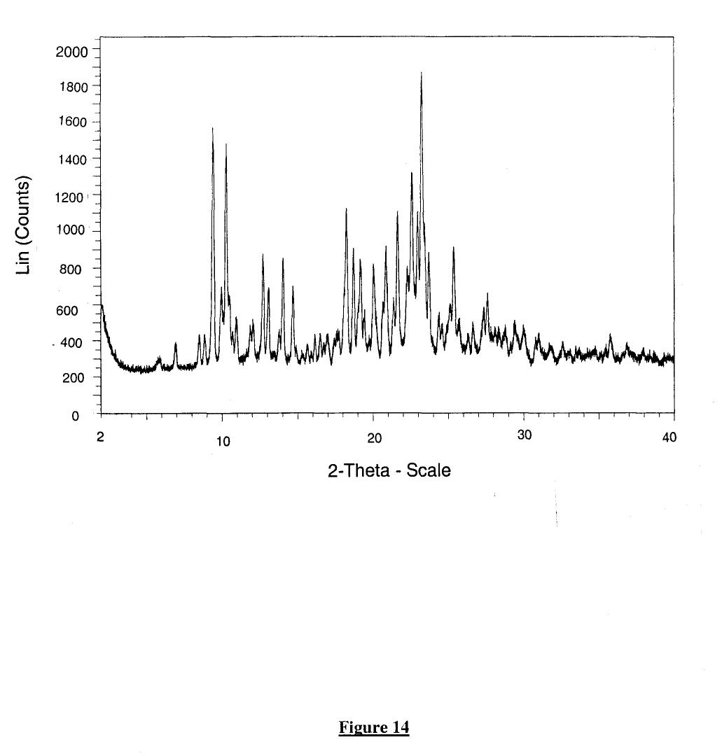

In one embodiment of the invention, there is provided a crystalline form of 1-[[5-(aminomethyl)-1-isopentyl-benzimidazol-2-yl]methyl]-3-cyclopropyl-4H-quinazolin-2-one adipate salt, Form C, which has an X-ray powder diffraction pattern substantially as shown in FIG. 14 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form A, which has an X-ray powder diffraction pattern with at least one specific peak at a 2-theta value of about 9.3, 17.7, 5.3, 16.9, 13.5, 17.2, 22.0, 14.5, 8.8 or 24.2 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form A, which has an X-ray powder diffraction pattern with specific peaks at 2-theta values of about 9.3, 17.7, 5.3, 16.9, 13.5, 17.2, 22.0, 14.5, 8.8 and 24.2 when measured using CuKa radiation, more particularly wherein said value may be plus or minus 0.5° 2-theta.

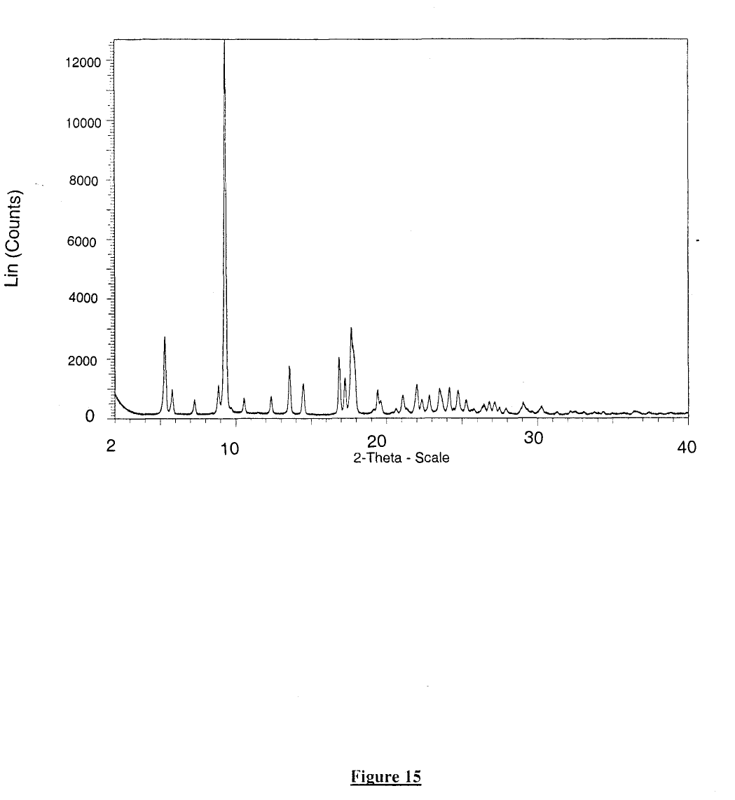

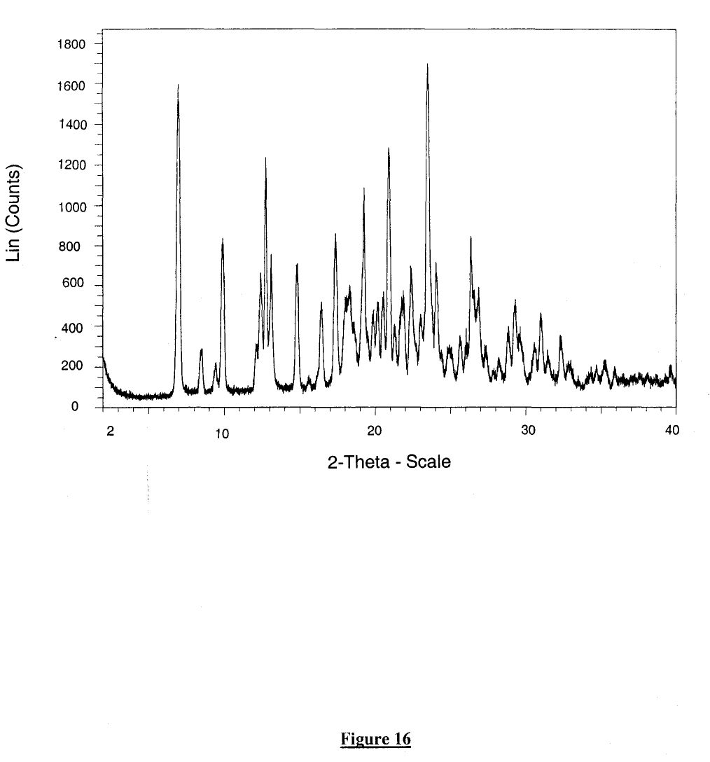

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form A, which has an X-ray powder diffraction pattern substantially as shown in FIG. 15 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form B, which has an X-ray powder diffraction pattern with at least one specific peak at a 2-theta value of about 23.5, 7.0, 20.9, 12.7, 19.3, 26.4, 17.4, 9.9, 24.1 or 14.8 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form B, which has an X-ray powder diffraction pattern with specific peaks at 2-theta values of about 23.5, 7.0, 20.9, 12.7, 19.3, 26.4, 17.4, 9.9, 24.1 and 14.8 when measured using CuKa radiation, more particularly wherein said value may be plus or minus 0.5° 2-theta.

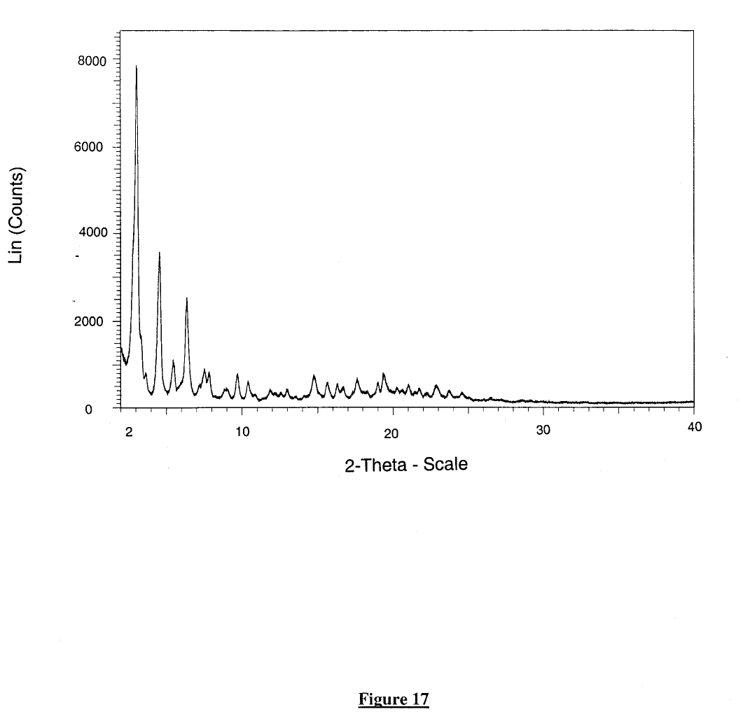

In one embodiment of the invention, there is provided a crystalline form of 1-[[5-(aminomethyl)-1-isopentyl-benzimidazol-2-yl]methyl]-3-cyclopropyl-4H-quinazolin-2-one cinnamate salt, Form B, which has an X-ray powder diffraction pattern substantially as shown in FIG. 16 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form A, which has an X-ray powder diffraction pattern with at least one specific peak at a 2-theta value of about 3.0, 4.5, 6.3, 5.4, 7.5, 7.8, 9.7, 19.4, 14.7 or 10.4 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form A, which has an X-ray powder diffraction pattern with specific peaks at 2-theta values of about 3.0, 4.5, 6.3, 5.4, 7.5, 7.8, 9.7, 19.4, 14.7 and 10.4 when measured using CuKa radiation, more particularly wherein said value may be plus or minus 0.5° 2-theta.

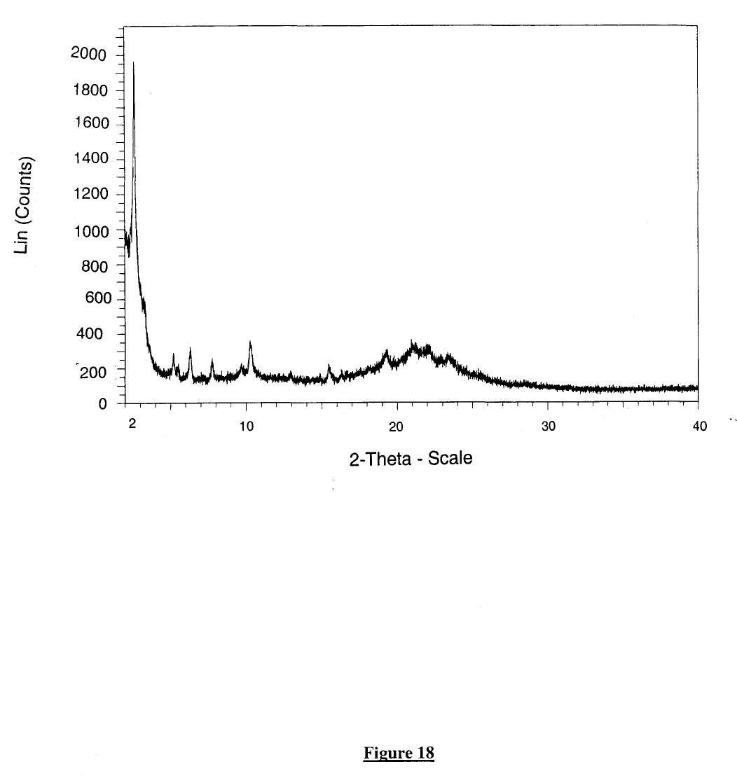

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form A, which has an X-ray powder diffraction pattern substantially as shown in FIG. 17 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form B, which has an X-ray powder diffraction pattern with at least one specific peak at a 2-theta value of about 2.6, 10.2, 6.3, 5.1, 7.7, 15.4 or 5.4 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form B, which has an X-ray powder diffraction pattern with specific peaks at 2-theta values of about 2.6, 10.2, 6.3, 5.1, 7.7, 15.4 and 5.4 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form B, which has an X-ray powder diffraction pattern substantially as shown in FIG. 18 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form C, which has an X-ray powder diffraction pattern with at least one specific peak at a 2-theta value of about 3.6, 5.5, 23.3, 8.4 or 10.2 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form C, which has an X-ray powder diffraction pattern with specific peaks at 2-theta values of about 3.6, 5.5, 23.3, 8.4 and 10.2 when measured using CuKa radiation, more particularly wherein said value may be plus or minus 0.5° 2-theta.

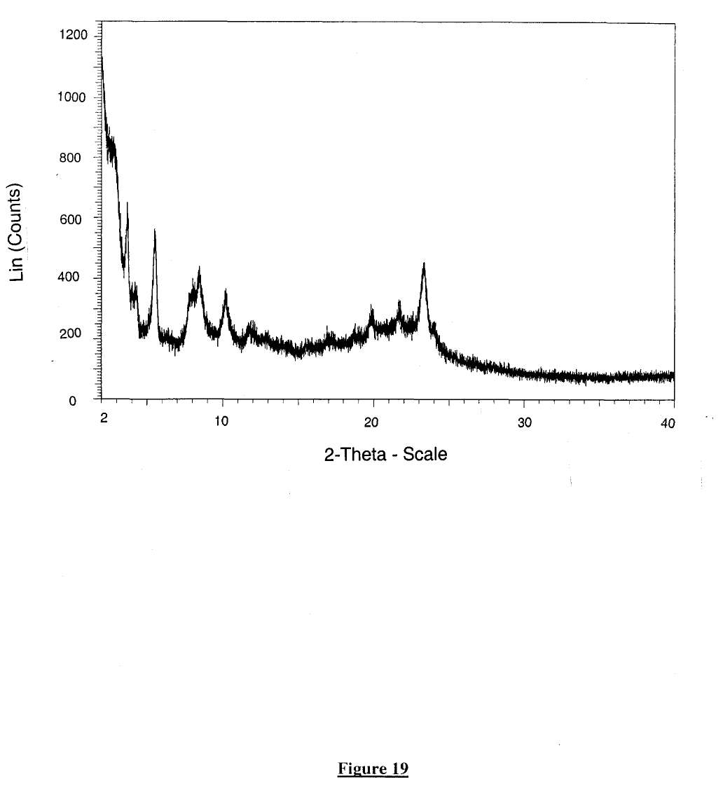

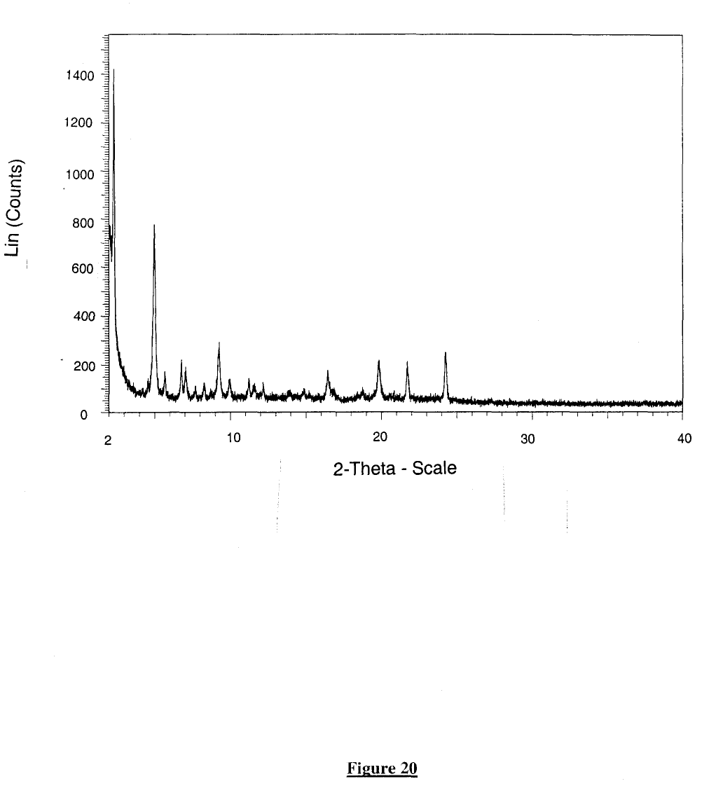

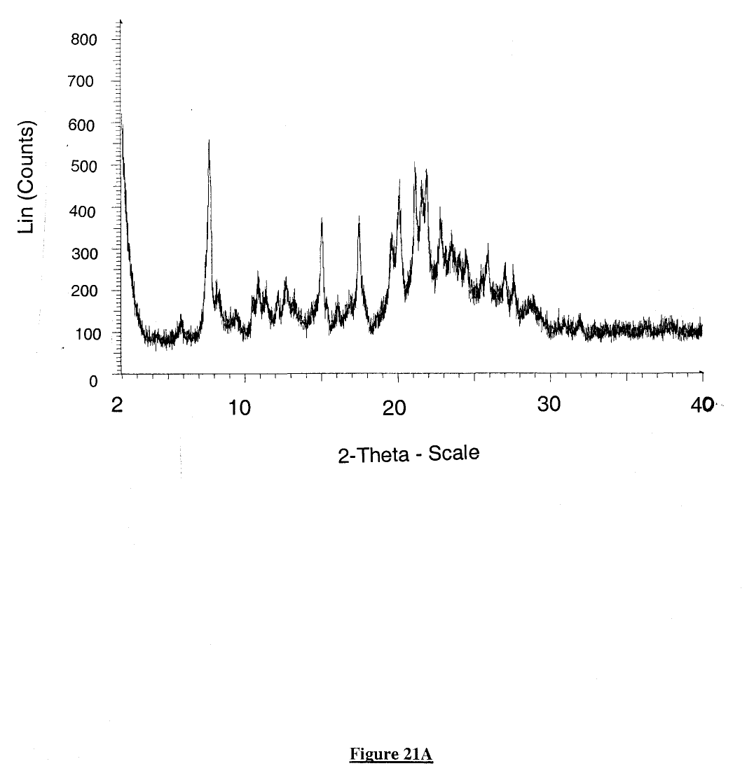

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form C, which has an X-ray powder diffraction pattern substantially as shown in FIG. 19 when measured using CuKa radiation.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form D, which has an X-ray powder diffraction pattern with at least one specific peak at a 2-theta value of about 2.3, 4.9, 9.2, 24.2, 6.7, 21.7, 19.8, 7.0, 16.4 or 5.6 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form D, which has an X-ray powder diffraction pattern with specific peaks at 2-theta values of about 2.3, 4.9, 9.2, 24.2, 6.7, 21.7, 19.8, 7.0, 16.4 and 5.6 when measured using CuKa radiation, more particularly wherein said value may be plus or minus 0.5° 2-theta.

In one embodiment of the invention, there is provided a crystalline form of 1-[[5-(aminomethyl)-1-isopentyl-benzimidazol-2-yl]methyl]-3-cyclopropyl-4H-quinazolin-2-one stearate salt, Form D, which has an X-ray powder diffraction pattern substantially as shown in FIG. 20 when measured using CuKa radiation.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form A, which has an X-ray powder diffraction pattern with at least one specific peak at a 2-theta value of 7.7, 21.2, 21.9, 20.1, 22.8, 15.0 or 17.4° when measured using CuKa radiation, more particularly wherein said value may be plus or minus 0.5° 2-theta.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form A, which has an X-ray powder diffraction pattern with specific peaks at 2-theta values of 7.7, 21.2, 21.9, 20.1, 22.8, 15.0 and 17.4° when measured using CuKa radiation, more particularly wherein said value may be plus or minus 0.5° 2-theta.

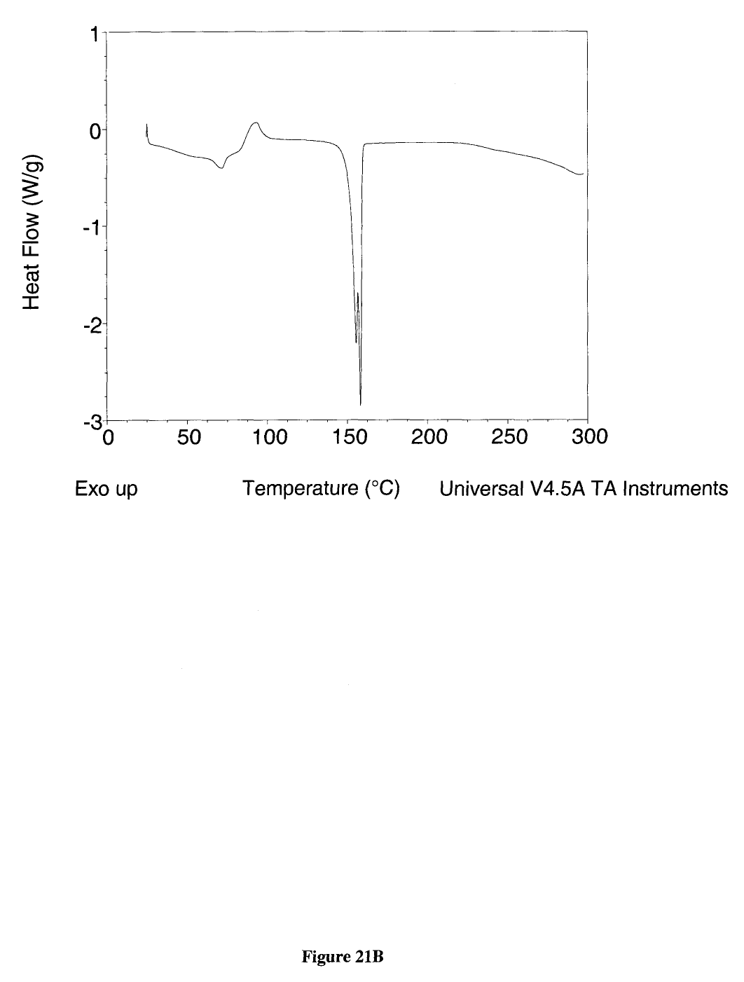

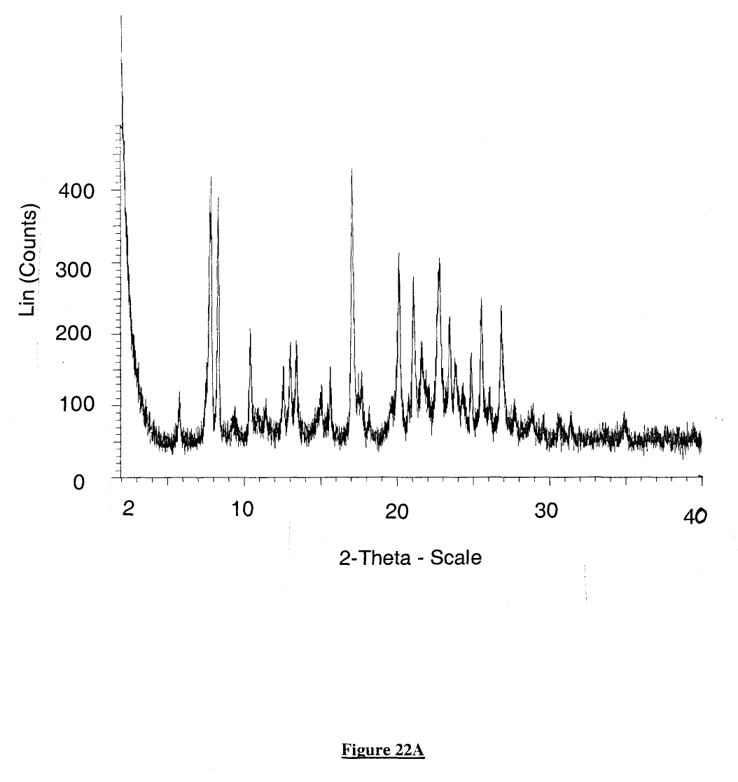

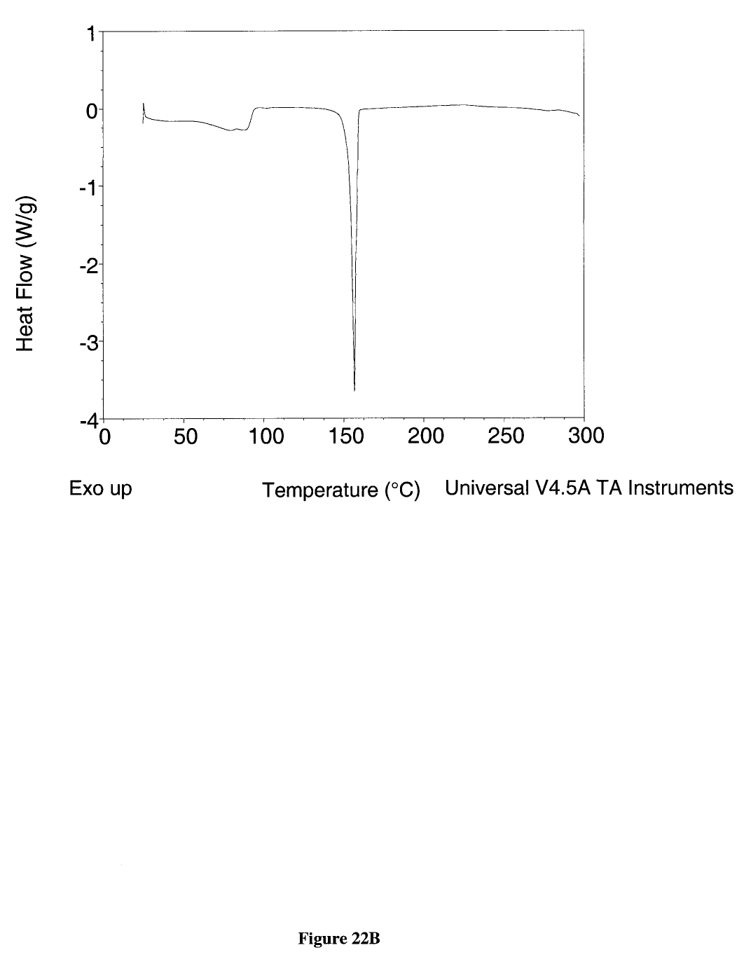

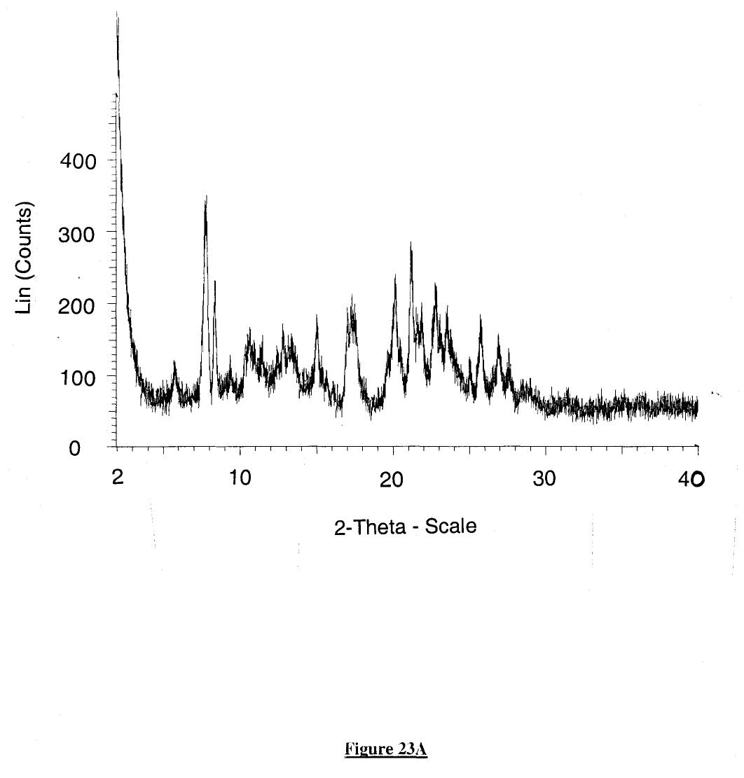

In one embodiment of the invention, there is provided crystalline form of 1-[[5-(aminomethyl)-1-(4,4,4-trifluorobutyl)benzimidazol-2-yl]methyl]-3-cyclopropyl-4H-quinazolin-2-one, Form A which has an X-ray powder diffraction pattern substantially the same as the X-ray powder diffraction pattern shown in FIG. 21A when measured using CuKa radiation.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form B, which has an X-ray powder diffraction pattern with at least one specific peak at a 2-theta value of about 17.1, 7.8, 8.3, 20.1, 22.8, 21.1, 10.4, 13.4, 13.0 or 12.6° when measured using CuKa radiation, more particularly wherein said value may be plus or minus 0.5° 2-theta.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form B, which has an X-ray powder diffraction pattern with specific peaks at 2-theta values of about 17.1, 7.8, 8.3, 20.1, 22.8, 21.1, 10.4, 13.4, 13.0 and 12.6° when measured using CuKa radiation, more particularly wherein said value may be plus or minus 0.5° 2-theta.

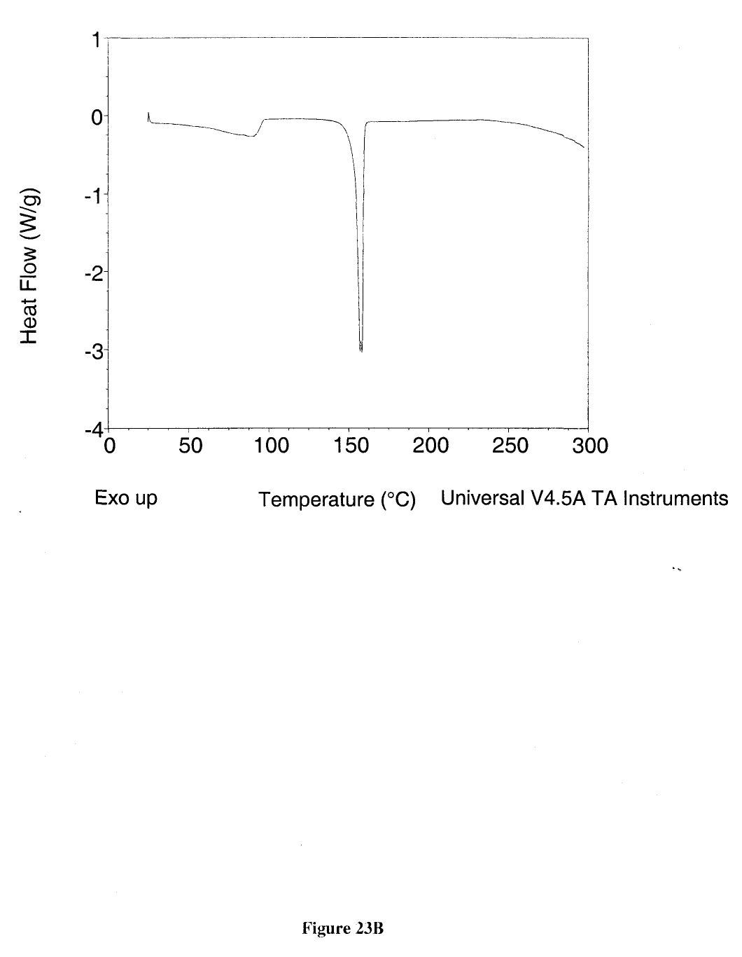

In one embodiment of the invention, there is provided crystalline form of 1-[[5-(aminomethyl)-1-(4,4,4-trifluorobutyl)benzimidazol-2-yl]methyl]-3-cyclopropyl-4H-quinazolin-2-one, Form B which has an X-ray powder diffraction pattern substantially the same as the X-ray powder diffraction pattern shown in FIG. 22A when measured using CuKa radiation.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form C, which has an X-ray powder diffraction pattern with specific peaks at 2-theta values of about 7.8, 21.2, 20.1, 8.3, 15.0, 23.5 and 25.7° when measured using CuKa radiation, more particularly wherein said value may be plus or minus 0.5° 2-theta.

In one embodiment of the invention, there is provided a crystalline form of 1-[[5-(aminomethyl)-1-(4,4,4-trifluorobutyl)benzimidazol-2-yl]methyl]-3-cyclopropyl-4H-quinazolin-2-one, Form C, which has an X-ray powder diffraction pattern with at least one specific peak at a 2-theta value of about 7.8, 21.2, 20.1, 8.3, 15.0, 23.5 or 25.7° when measured using CuKa radiation, more particularly wherein said value may be plus or minus 0.5° 2-theta.

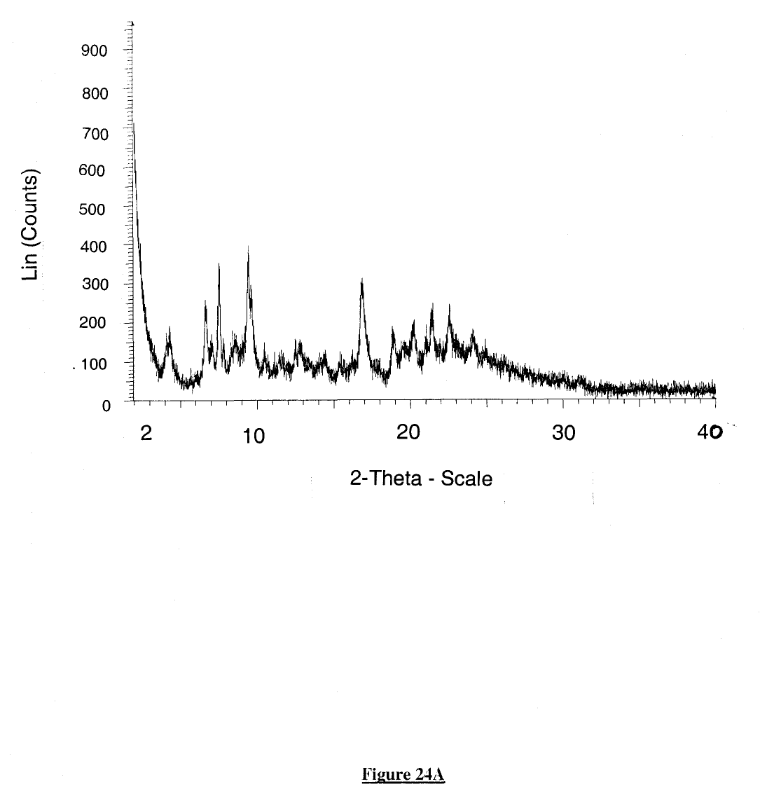

In one embodiment of the invention, there is provided crystalline form of 1-[[5-(aminomethyl)-1-(4,4,4-trifluorobutyl)benzimidazol-2-yl]methyl]-3-cyclopropyl-4H-quinazolin-2-one, Form C which has an X-ray powder diffraction pattern substantially the same as the X-ray powder diffraction pattern shown in FIG. 23A when measured using CuKa radiation.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one IPA solvate, which has an X-ray powder diffraction pattern with at least one specific peak at a 2-theta value of about 9.4, 7.5, 16.8, 6.6, 22.6, 20.2, 4.3, 24.1, 18.9 or 8.5° when measured using CuKa radiation, more particularly wherein said value may be plus or minus 0.5° 2-theta.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one IPA solvate, which has an X-ray powder diffraction pattern with specific is peaks at 2-theta values of about 9.4, 7.5, 16.8, 6.6, 22.6, 20.2, 4.3, 24.1, 18.9 and 8.5° when measured using CuKa radiation, more particularly wherein said value may be plus or minus 0.5° 2-theta.

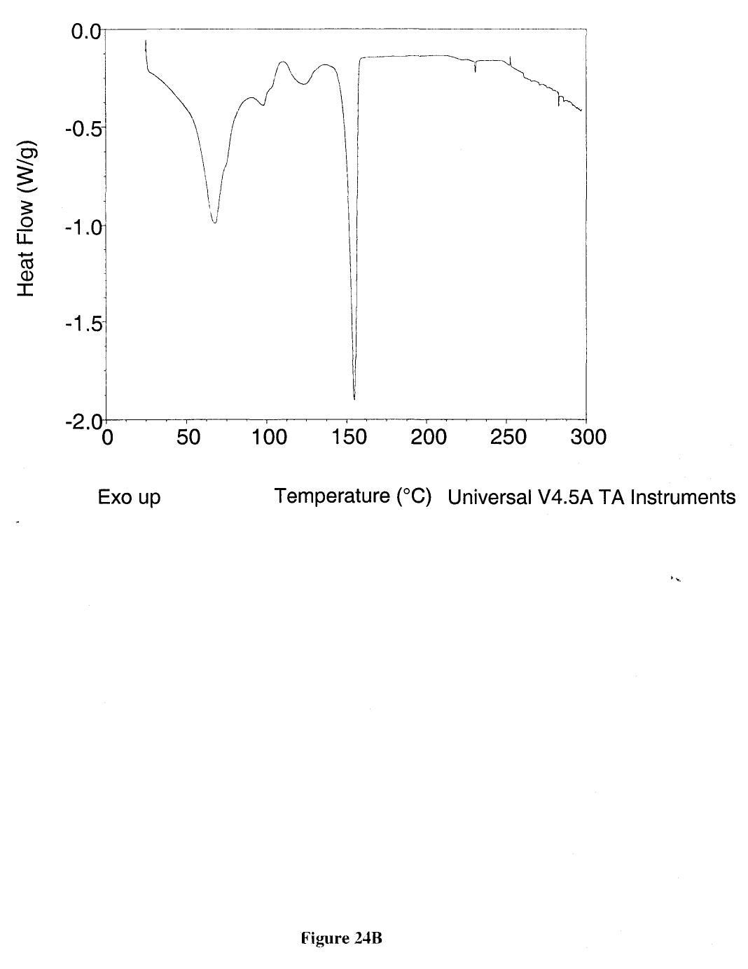

In one embodiment of the invention, there is provided crystalline form, 1-[[5-(aminomethyl)-1-(4,4,4-trifluorobutyl)benzimidazol-2-yl]methyl]-3-cyclopropyl-4H-quinazolin-2-one IPA solvate which has an X-ray powder diffraction pattern substantially the same as the X-ray powder diffraction pattern shown in FIG. 24A when measured using CuKa radiation.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one acetonitrile solvate, which has an X-ray powder diffraction pattern with at least one specific peak at a 2-theta value of about 21.6, 23.5, 7.0, 19.5, 9.5, 17.1, 9.0, 7.5 or 13.0° when measured using CuKa radiation, more particularly wherein said value may be plus or minus 0.5° 2-theta.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one acetonitrile solvate, which has an X-ray powder diffraction pattern with specific peaks at 2-theta values of about 21.6, 23.5, 7.0, 19.5, 9.5, 17.1, 9.0, 7.5 and 13.0° when measured using CuKa radiation, more particularly wherein said value may be plus or minus 0.5° 2-theta.

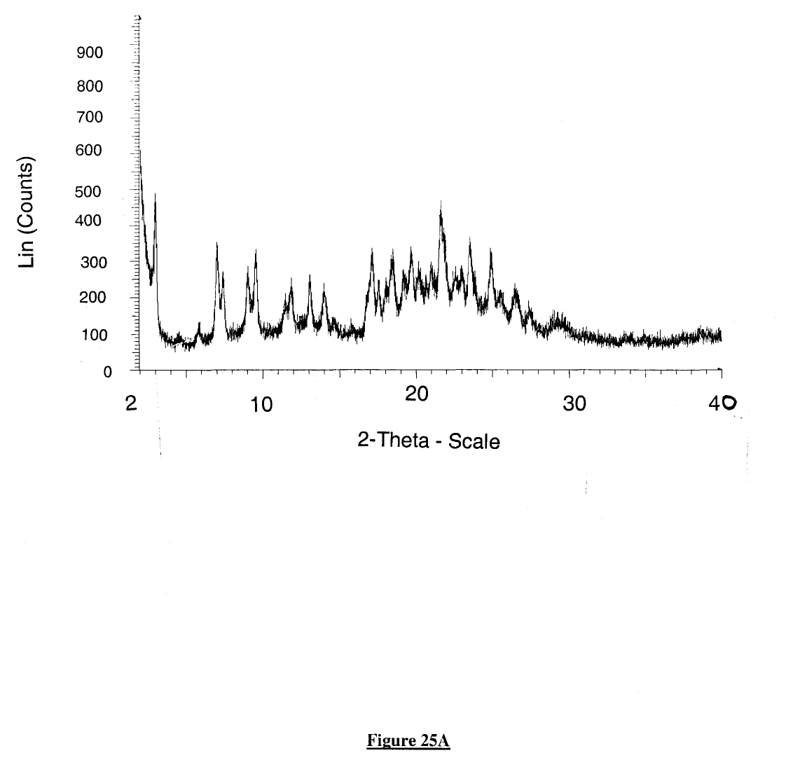

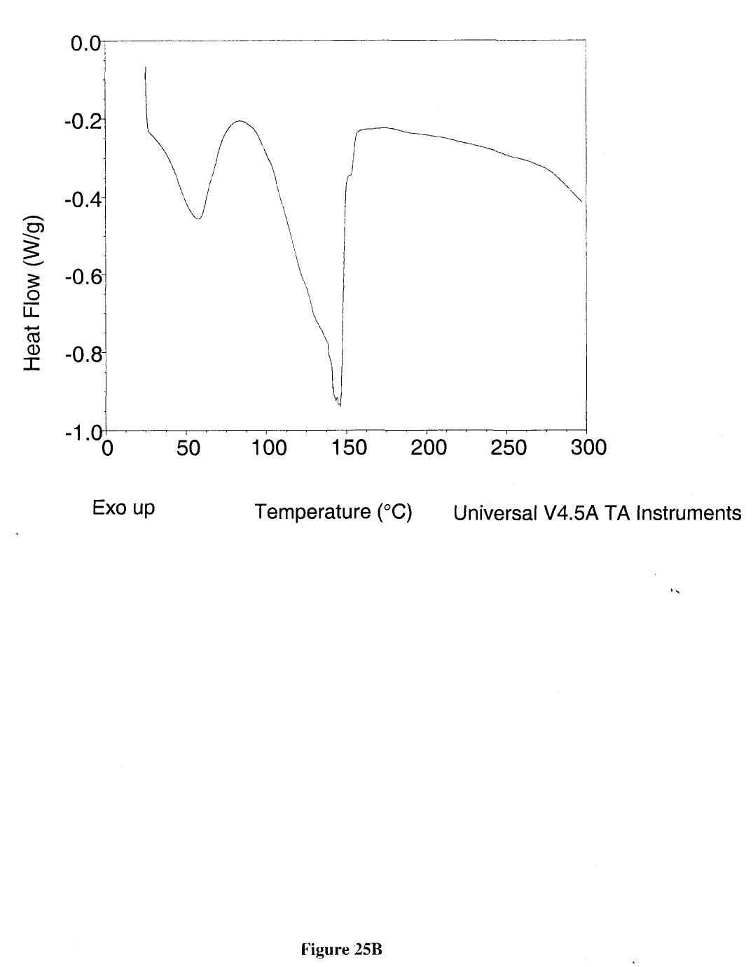

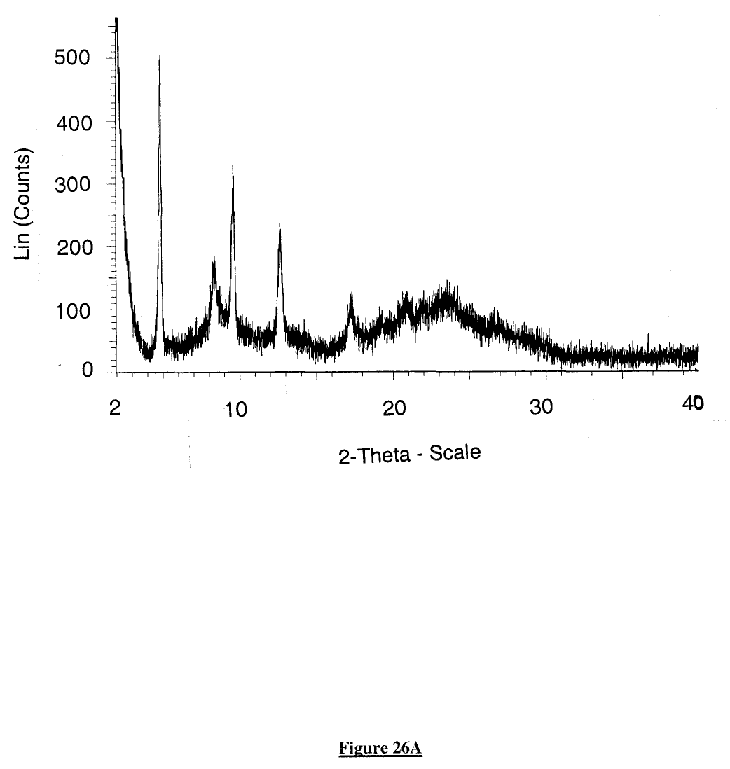

In one embodiment of the invention, there is provided crystalline form, 1-[[5-(aminomethyl)-1-(4,4,4-trifluorobutyl)benzimidazol-2-yl]methyl]-3-cyclopropyl-4H-quinazolin-2-one acetonitrile solvate which has an X-ray powder diffraction pattern substantially the same as the X-ray powder diffraction pattern shown in FIG. 25A when measured using CuKa radiation.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one methanol solvate, which has an X-ray powder diffraction pattern with at least one specific peak at a 2-theta value of about 4.8, 9.5, 12.6 or 8.3° when measured using CuKa radiation, more particularly wherein said value may be plus or minus 0.5° 2-theta.

In one embodiment of the invention, there is provided a crystalline form, 1-[[5-(aminomethyl)-1-(4,4,4-trifluorobutyl)benzimidazol-2-yl]methyl]-3-cyclopropyl-4H-quinazolin-2-one methanol solvate, which has an X-ray powder diffraction pattern with specific peaks at 2-theta values of about 4.8, 9.5, 12.6 and 8.3° when measured using CuKa radiation, more particularly wherein said value may be plus or minus 0.5° 2-theta.

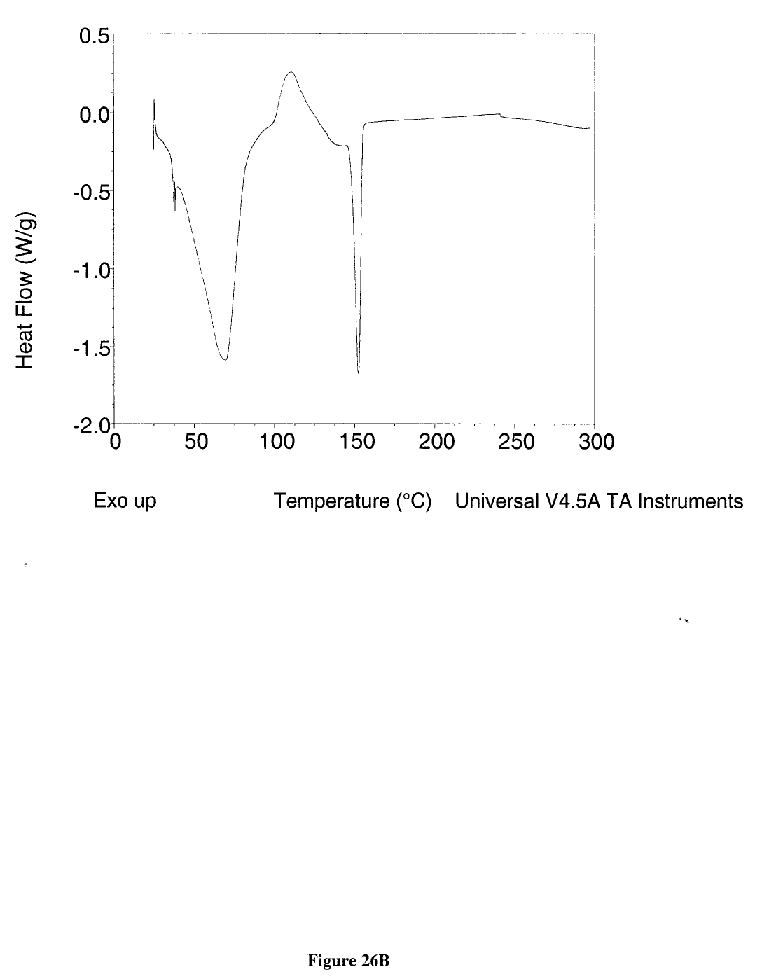

In one embodiment of the invention, there is provided crystalline form, 1-[[5-(aminomethyl)-1-(4,4,4-trifluorobutyl)benzimidazol-2-yl]methyl]-3-cyclopropyl-4H-quinazolin-2-one methanol solvate which has an X-ray powder diffraction pattern substantially the same as the X-ray powder diffraction pattern shown in FIG. 26A when measured using CuKa radiation.

When it is stated that the invention relates to a crystalline form the degree of crystallinity is conveniently greater than about 60%, more conveniently greater than about 80%, preferably greater than about 90% and more preferably greater than about 95%. Most preferably the degree of crystallinity is greater than about 98%.

It will be understood that the 2-theta values of the X-ray powder diffraction pattern may vary slightly from one machine to another or from one sample to another, and so the values quoted are not to be construed as absolute.

It is known in the art that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions and sample preparation. For example, persons skilled in the art of X-ray powder diffraction will realise that the relative intensities of peaks may vary according to the orientation of the sample under test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern data presented herein is not to be construed as absolute and any crystalline form that provides a power diffraction pattern substantially identical to those disclosed herein fall within the scope of the present disclosure (for further information see Jenkins, R & Snyder, R. L. ‘Introduction to X-Ray Powder Diffractometry’ John Wiley & Sons, 1996).

Generally, a measurement error of a diffraction angle in an X-ray powder diffractogram is approximately plus or minus 0.5° 2-theta, and such degree of a measurement error should be taken into account when considering the X-ray powder diffraction pattern in FIGS. 1A to 9A, 10 to 20 and 21A to 26A when reading Tables 1 to 26. Furthermore, it should be understood that intensities might fluctuate depending on experimental conditions and sample preparation (preferred orientation). Preferred orientation occurs when there is a tendency for the crystal morphology (shape) to exhibit a particular orientation such as acicular (needle-like), resulting in a non-random orientation of the crystals when sampled for XRPD analysis. This can result in differences in relative intensity of peaks.

The present invention further provides a process for the preparation of a compound of formula (I) or a pharmaceutically acceptable salt thereof as defined above which comprises,

(a) reacting a compound of formula (II)

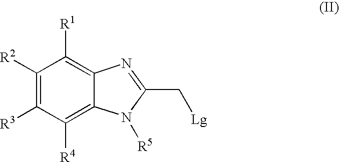

wherein Lg represents a suitable leaving group, such as chloro, bromo, iodo, mesylate or tosylate (particularly chloro), R¹, R³, R⁴ and R⁵ are as defined for formula (I) and R² is as defined for formula (I) or a protected derivative thereof,

with a compound of formula (III)

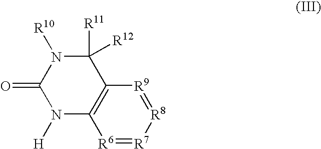

wherein R⁶, R⁷, R⁸, R⁹, R¹⁰, R¹¹ and R¹² are as defined in formula (I); or

(b) when R² represents CH₂NH₂, reduction of a compound of formula (I) wherein R² represents CN

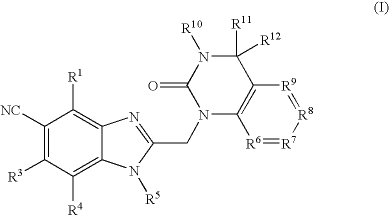

wherein R¹, R³, R⁴, R⁵, R⁶, R⁷, R⁸, R⁹, R¹⁰, R¹¹ and R¹² are as defined in formula (I);

and optionally after (a) or (b) carrying out one or more of the following:
converting the compound obtained to a further compound of the invention
forming a pharmaceutically acceptable salt of the compound
removing any protecting group that is present (by conventional means)
preparing a crystalline form thereof.

In process (a), the coupling reaction may be carried out by reaction of a chloromethyl derivative (II) with the quinazolinone (III) in the presence of a suitable base such as sodium hydride or cesium carbonate or sodium tert-pentoxide. Such processes are well known in the literature and will be readily apparent to the skilled man. In a particular embodiment of process (a), the group R² represents the protected moiety tert-BocNHCH₂—.

Specific processes for the preparation of compounds of formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention.

Compounds of formula (I) can be converted into further compounds of formula (I) using standard procedures. Thus, compounds of formula (I) wherein R² represents CN may be converted into further compounds of formula (I) wherein R² represents CH₂NH₂ by the reduction of a compound of formula (I) wherein R² represents CN

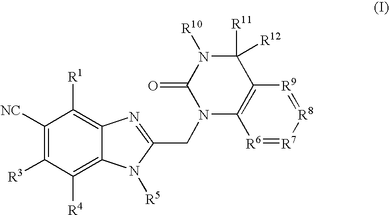

wherein R₁, R³, R⁴, R⁵, R⁶, R⁷, R⁸, R⁹, R¹⁰, R¹¹ and R¹² are as defined in formula (I) (i.e. according to process (b) above). The reduction may be effected by known methods, for example, using catalytic hydrogenation, for example using hydrogen and a palladium or platinum catalyst, in a suitable solvent such as methanol and at a suitable temperature.

Compounds of formula (I) wherein R² represents CN may be converted into further compounds of formula (I) wherein R² represents C(═NOH)NH₂, which compounds may in turn be converted into compounds wherein R² represents C(═NH)NH₂. Compounds of formula (I) wherein R² represents CN may be converted into further compounds of formula (I) wherein R² represents CH₂NH(CH₂)₃NH₂, which compounds may in turn be converted into compounds wherein R² represents C(═NH)NH₂. Such transformations may be achieved using methods that will be readily apparent to the man skilled in the art. Certain intermediates may be novel. Such novel intermediates form another aspect of the invention.

It will be appreciated by those skilled in the art that in the processes of the present invention certain functional groups such as hydroxyl or amino groups may need to be protected by protecting groups. Thus, the preparation of the compounds of formula (I) may involve, at an appropriate stage, the addition and/or removal of one or more protecting groups.

The protecting groups used in the processes above may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1 to 4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); lower alkanoyloxyalkyl groups (for example pivaloyloxymethyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl. For example a tert butoxycarbonyl protecting group may be removed from an amino group by an acid catalysed hydrolysis, for example using trifluoroacetic acid.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted into a pharmaceutically acceptable salt thereof, preferably an acid addition salt such as a hydrochloride, hydrobromide, sulfate, phosphate, acetate, fumarate, maleate, tartrate, lactate, citrate, pyruvate, succinate, oxalate, methanesulfonate, p-toluenesulfonate, adipate, ascorbate, besylate, benzoate, cinnamate, ethanedisulfonate, glutarate, glycolate, napadisylate, oleate or stearate salt. References herein to compounds of formula (I) include pharmaceutically acceptable salts of the compounds of formula (I).

In one embodiment, the acid addition salt may be an acetate, adipate, ascorbate, besylate, benzoate, cinnamate, citrate, ethanedisulfonate, glutarate, glycolate, hydrochloride, lactate, napadisylate, oleate, phosphate, stearate, sulfate, tartrate or p-toluenesulfonate salt, particularly an acetate, adipate, cinnamate or stearate salt. In another embodiment, the acid addition salt is an acetate salt. In another embodiment, the acid addition salt is an adipate salt. In another embodiment, the acid addition salt is a cinnamate salt. In another embodiment, the acid addition salt is a stearate salt.

According to a further aspect of the present invention, there is provided 1-[[5-(aminomethyl)-1-isopentyl-benzimidazol-2-yl]methyl]-3-cyclopropyl-4H-quinazolin-2-one acetate salt.

According to a further aspect of the present invention, there is provided 1-[[5-(aminomethyl)-1-isopentyl-benzimidazol-2-yl]methyl]-3-cyclopropyl-4H-quinazolin-2-one adipate salt.

According to a further aspect of the present invention, there is provided 1-[[5-(aminomethyl)-1-isopentyl-benzimidazol-2-yl]methyl]-3-cyclopropyl-4H-quinazolin-2-one cinnamate salt.

According to a further aspect of the present invention, there is provided 1-[[5-(aminomethyl)-1-isopentyl-benzimidazol-2-yl]methyl]-3-cyclopropyl-4H-quinazolin-2-one stearate salt.

The compounds of formula (I) and their pharmaceutically acceptable salts have activity as pharmaceuticals, in particular as antiviral agents.

More particularly, the compounds of formula (I) and their pharmaceutically acceptable salts may be used in the treatment of RSV. The compounds have the additional advantage that they exhibit improved solubility when compared to similar compounds known in the art.

Thus, the present invention provides a compound of formula (I) or a pharmaceutically acceptable salt thereof as hereinbefore defined for use in therapy.

The present invention provides a compound of the Formula (I) or a pharmaceutically acceptable salt thereof as hereinbefore defined for use as a medicament.

In a further aspect, the present invention provides the use of a compound of formula (I) or a pharmaceutically acceptable salt thereof as hereinbefore defined in the manufacture of a medicament for use in therapy.

In a further aspect, the present invention provides the use of a compound of formula (I) or a pharmaceutically acceptable salt thereof as hereinbefore defined in the manufacture of a medicament for the treatment of RSV.

In a further aspect, the present invention provides a compound of formula (I) or a pharmaceutically acceptable salt thereof as hereinbefore defined for the treatment of RSV.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, RSV which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. The daily dosage of the compound of the invention may be in the range from 0.01 mg/kg to 650 mg/kg. A unit dose form such as a tablet or a capsule will usually contain 1-250 mg of active ingredient. For example, a compound of formula (I), such as 1-[[5-(aminomethyl)-1-isopentyl-benzimidazol-2-yl]methyl]-3-cyclopropyl-4H-quinazolin-2-one, could be administered to a human patient at a dose of between 100-250 mg either once a day, twice or three times a day. For example, a compound of formula (I), such as 1-[[5-(aminomethyl)-1-(4,4,4-trifluorobutyl)benzimidazol-2-yl]methyl]-3-cyclopropyl-4H-quinazolin-2-one, could be administered to a human patient at a dose of between 100-250 mg either once a day, twice or three times a day.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, solutions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulfates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride. Further suitable carriers for suspensions include sterile water, hydroxypropylmethyl cellulose (HPMC), polysorbate 80, polyvinylpyrrolidone (PVP), aerosol AOT (i.e. sodium 1,2-bis(2-ethylhexoxycarbonyl)ethanesulphonate), pluronic F127 and/or captisol (i.e. sulfobutylether-beta-cyclodextrin).

For example, the compounds of the invention may be formulated as aqueous suspensions in one of the following carriers:
(i) 0.5% w/v hydroxypropylmethyl cellulose (HPMC)/0.1% w/v polysorbate 80
(ii) 0.67% w/v polyvinylpyrrolidone (PVP)/0.33% w/v aerosol AOT (sodium 1,2-bis(2-ethylhexoxycarbonyl)ethanesulphonate)
(iii) 1% w/v pluronic F127
(iv) 0.5% w/v polysorbate 80

The carriers may be prepared by standard procedures known to persons skilled in the art. For example, each of the carriers (i) to (iv) may be prepared by weighing the required amount of excipient into a suitable vessel, adding approximately 80% of the final volume of water and magnetically stirring until a solution is formed. The carrier is then made up to volume with water. The aqueous suspensions of compounds of formula I may be prepared by weighing the required amount of a compound of formula I into a suitable vessel, adding 100% of the required volume of carrier and magnetically stirring.

The compounds of the invention may be formulated as solutions in suitable carriers/solvents. For example, compounds of formula I (such as 1-[[5-(aminomethyl)-1-isopentyl-benzimidazol-2-yl]methyl]-3-cyclopropyl-4H-quinazolin-2-one) may be formulated as an aqueous solution in 30% w/v captisol (i.e. sulfobutylether-beta-cyclodextrin) at pH4.

A carrier of 30% w/v captisol (i.e. sulfobutylether-beta-cyclodextrin) may be prepared by weighing the required amount of captisol into a suitable vessel, adding approximately 80% of the final volume of water and magnetically stirring until a solution is formed. The carrier may then be made up to volume with water.

An aqueous solution of a compound of formula I (such as 1-[[5-(aminomethyl)-1-isopentyl-benzimidazol-2-yl]methyl]-3-cyclopropyl-4H-quinazolin-2-one) may be prepared by weighing the required amount of the compound into a suitable vessel and adding approximately 80% of the required volume of the carrier. Using an aqueous solution of hydrochloric acid, the pH may be adjusted to pH2 and the resulting mixture magnetically stirred until a solution is formed. The formulation may then be made up to volume with carrier and pH adjusted to pH4 using an aqueous solution of a suitable base, such as sodium hydroxide.

Solutions for injection or infusion may contain as carrier, for example, sterile water or preferably they may be in the form of sterile, aqueous, isotonic saline solutions.

The compounds of the invention may also be administered in conjunction with other compounds used for the treatment of viral infections.

Thus, the invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of a viral infection, particularly infection by RSV.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Suitable therapeutic agents for use in the combination therapies include (i) RSV nucleocapsid(N)-protein inhibitors, (ii), other protein inhibitors, such as those that inhibit the phosphoprotein (P) protein and large (L) protein; (iii) anti-RSV monoclonal antibodies, such as the F-protein antibodies, (iv) immunomodulating toll-like receptor compounds; (v) other respiratory virus anti-virals, such as anti-influenza and anti-rhinovirus compounds and/or (vi) anti-inflammatory compounds.

The RSV nucleocapsid(N)-protein plays a pivotal role in viral transcription and replication, mediating the interaction between the genomic RNA and the virally encoded RNA-dependent RNA polymerase. The RSV P- and L-proteins are components of RSV's virally encoded RNA-dependent RNA polymerase.

According to a further aspect of the present invention, a compound of the invention, or a pharmaceutically acceptable salt thereof, or a pharmaceutical composition or formulation comprising a compound of the invention, may be administered in conjunction with one or more further therapeutic agent(s) selected from:
(i) RSV N-protein inhibitors;
(ii) other RSV protein inhibitors, such as those that inhibit the phosphoprotein (P) protein and large (L) protein;
(iii) anti-RSV monoclonal antibodies, such as the F-protein targeting antibodies;
(iv) immunomodulating toll-like receptor compounds;
(v) other respiratory virus anti-virals, such as anti-influenza and anti-rhinovirus compounds; and
(vi) anti-inflammatory compounds.

According to a further aspect of the invention, there is provided a compound of the formula (I) or a pharmaceutically acceptable salt thereof as hereinbefore defined in combination with one or more of the therapeutic agents listed as (i) to (vi) above for use in the treatment of RSV.

According to a further aspect of the invention, there is provided 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above for use in the treatment of RSV.

According to a further aspect of the invention, there is provided 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above for use in the treatment of RSV.

According to a further aspect of the invention, there is provided the use of a compound of the formula (I) or a pharmaceutically acceptable salt thereof as hereinbefore defined in combination with one or more of the therapeutic agents listed as (i) to (vi) above in the manufacture of a medicament for use in the treatment of RSV.

According to a further aspect of the invention, there is provided the use of 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above in the manufacture of a medicament for use in the treatment of RSV.

According to a further aspect of the invention, there is provided the use of 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above in the manufacture of a medicament for use in the treatment of RSV.

According to a further aspect of the present invention, there is provided a method for the treatment of RSV in a warm-blooded animal, such as man, in need of such treatment which comprises administering to said animal an effective amount of a compound of formula (I) or a pharmaceutically acceptable salt thereof as hereinbefore defined in combination with a one or more of the therapeutic agents listed as (i) to (vi) above.

According to a further aspect of the present invention, there is provided a method for the treatment of RSV in a warm-blooded animal, such as man, in need of such treatment which comprises administering to said animal an effective amount of 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above.

According to a further aspect of the present invention, there is provided a method for the treatment of RSV in a warm-blooded animal, such as man, in need of such treatment which comprises administering to said animal an effective amount of 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above.

According to a further aspect of the invention, there is provided a compound of the formula (I) or a pharmaceutically acceptable salt thereof as hereinbefore defined in combination with a RSV N-protein inhibitor for use in the treatment of RSV.

According to a further aspect of the invention, there is provided 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor for use in the treatment of RSV.

According to a further aspect of the invention, there is provided 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor for use in the treatment of RSV.

According to a further aspect of the invention, there is provided the use of a compound of the formula (I) or a pharmaceutically acceptable salt thereof as hereinbefore defined in combination with a RSV N-protein inhibitor in the manufacture of a medicament for use in the treatment of RSV.

According to a further aspect of the invention, there is provided the use of 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor in the manufacture of a medicament for use in the treatment of RSV.

According to a further aspect of the invention, there is provided the use of 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor in the manufacture of a medicament for use in the treatment of RSV.

According to a further aspect of the present invention, there is provided a method for the treatment of RSV in a warm-blooded animal, such as man, in need of such treatment which comprises administering to said animal an effective amount of a compound of formula (I) or a pharmaceutically acceptable salt thereof as hereinbefore defined in combination with a RSV N-protein inhibitor.

According to a further aspect of the present invention, there is provided a method for the treatment of RSV in a warm-blooded animal, such as man, in need of such treatment which comprises administering to said animal an effective amount of 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor.

According to a further aspect of the present invention, there is provided a method for the treatment of RSV in a warm-blooded animal, such as man, in need of such treatment which comprises administering to said animal an effective amount of 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor.

According to a further aspect of the present invention, there is provided a pharmaceutical composition which comprises a compound of formula (I) or a pharmaceutically acceptable salt thereof as hereinbefore defined in combination with one or more of the therapeutic agents listed as (i) to (vi) above and in association with a pharmaceutically acceptable diluent or carrier.

According to a further aspect of the present invention, there is provided a pharmaceutical composition which comprises 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above and in association with a pharmaceutically acceptable diluent or carrier.

According to a further aspect of the present invention, there is provided a pharmaceutical composition which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above and in association with a pharmaceutically acceptable diluent or carrier.

According to a further aspect of the present invention, there is provided a pharmaceutical composition which comprises a compound of formula (I) or a pharmaceutically acceptable salt thereof as hereinbefore defined in combination with one or more of the therapeutic agents listed as (i) to (vi) above and in association with a pharmaceutically acceptable diluent or carrier for use in the treatment of RSV.

According to a further aspect of the present invention, there is provided a pharmaceutical composition which comprises 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above and in association with a pharmaceutically acceptable diluent or carrier for use in the treatment of RSV.

According to a further aspect of the present invention, there is provided a pharmaceutical composition which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above and in association with a pharmaceutically acceptable diluent or carrier for use in the treatment of RSV.

According to a further aspect of the present invention, there is provided a pharmaceutical composition which comprises a compound of formula (I) or a pharmaceutically acceptable salt thereof as hereinbefore defined in combination with a RSV N-protein inhibitor and in association with a pharmaceutically acceptable diluent or carrier.

According to a further aspect of the present invention, there is provided a pharmaceutical composition which comprises 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor and in association with a pharmaceutically acceptable diluent or carrier.

According to a further aspect of the present invention, there is provided a pharmaceutical composition which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor and in association with a pharmaceutically acceptable diluent or carrier.

According to a further aspect of the present invention, there is provided a pharmaceutical composition which comprises a compound of formula (I) or a pharmaceutically acceptable salt thereof as hereinbefore defined in combination with a RSV N-protein inhibitor and in association with a pharmaceutically acceptable diluent or carrier for use in the treatment of RSV.

According to a further aspect of the present invention, there is provided a pharmaceutical composition which comprises 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor and in association with a pharmaceutically acceptable diluent or carrier for use in the treatment of RSV.

According to a further aspect of the present invention, there is provided a pharmaceutical composition which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor and in association with a pharmaceutically acceptable diluent or carrier for use in the treatment of RSV.

According to a further aspect of the present invention, there is provided a kit which comprises a compound of formula (I) or a pharmaceutically acceptable salt thereof as hereinbefore defined in combination with one or more of the therapeutic agents listed as (i) to (vi) above.

According to a further aspect of the present invention, there is provided a kit which comprises 1-[[5-(aminomethyl)-1-isopentyl-benzimidazol-2-yl]methyl]-3-cyclopropyl-4H-quinazolin-2-one or a pharmaceutically acceptable salt thereof in combination with one or more of the therapeutic agents listed as (i) to (vi) above.

According to a further aspect of the present invention, there is provided a kit which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with one or more of the therapeutic agents listed as (i) to (vi) above.

According to a further aspect of the present invention, there is provided a kit which is comprises (a) a compound of formula (I) or a pharmaceutically acceptable salt thereof as hereinbefore defined in a first unit dosage form, (b) one or more of the therapeutic agents listed as (i) to (vi) above in a second unit dosage form, and (c) container means for containing said first and second dosage forms.

According to a further aspect of the present invention, there is provided a kit which comprises (a) 1-[[5-(aminomethyl)-1-isopentyl-benzimidazol-2-yl]methyl]-3-cyclopropyl-4H-quinazolin-2-one or a pharmaceutically acceptable salt thereof in a first unit dosage form, (b) one or more of the therapeutic agents listed as (i) to (vi) above in a second unit dosage form, and (c) container means for containing said first and second dosage forms.

According to a further aspect of the present invention, there is provided a kit which comprises (a) 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof, (b) one or more of the therapeutic agents listed as (i) to (vi) above in a second unit dosage form, and (c) container means for containing said first and second dosage forms.

According to a further aspect of the present invention, there is provided a kit which comprises a compound of formula (I) or a pharmaceutically acceptable salt thereof as hereinbefore defined in combination with a RSV N-protein inhibitor.

According to a further aspect of the present invention, there is provided a kit which comprises 1-[[5-(aminomethyl)-1-isopentyl-benzimidazol-2-yl]methyl]-3-cyclopropyl-4H-quinazolin-2-one or a pharmaceutically acceptable salt thereof in combination with a RSV N-protein inhibitor.

According to a further aspect of the present invention, there is provided a kit which comprises 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof in combination with a RSV N-protein inhibitor.

According to a further aspect of the present invention, there is provided a kit which comprises (a) a compound of formula (I) or a pharmaceutically acceptable salt thereof as hereinbefore defined in a first unit dosage form, (b) a RSV N-protein inhibitor in a second unit dosage form, and (c) container means for containing said first and second dosage forms.

According to a further aspect of the present invention, there is provided a kit which comprises (a) 1-[[5-(aminomethyl)-1-isopentyl-benzimidazol-2-yl]methyl]-3-cyclopropyl-4H-quinazolin-2-one or a pharmaceutically acceptable salt thereof in a first unit dosage form, (b) a RSV N-protein inhibitor in a second unit dosage form, and (c) container means for containing said first and second dosage forms.

According to a further aspect of the present invention, there is provided a kit which comprises (a) 1-[[5-(aminomethyl)-1-(4,4,4-trifluorobutyl)benzimidazol-2-yl]methyl]-3-cyclopropyl-4H-quinazolin-2-one or a pharmaceutically acceptable salt thereof, (b) a RSV N-protein inhibitor in a second unit dosage form, and (c) container means for containing said first and second dosage forms.

In the combinations discussed herein, any suitable RSV N-protein inhibitors, other RSV protein inhibitors, such as those that inhibit the phosphoprotein (P) protein and large (L) protein, anti-RSV monoclonal antibodies, such as the F-protein targeting antibodies, immunomodulating toll-like receptor compounds, other respiratory virus anti-virals, such as anti-influenza and anti-rhinovirus compounds, and/or anti-inflammatory compounds may be used, as would be appreciated by persons skilled in the art.

In the combinations discussed herein, any suitable RSV N-protein inhibitor may be used. Examples of suitable RSV N-protein inhibitors are described in WO-2004/026843, any of which inhibitors may be included in a combination treatment as discussed herein. In particular, an example of a suitable RSV N-protein inhibitor for combination with the compounds of formula (I) or a pharmaceutically acceptable salt thereof as hereinbefore defined (for example 1-[[5-(aminomethyl)-1-isopentyl-benzimidazol-2-yl]methyl]-3-cyclopropyl-4H-quinazolin-2-one or 1-[[5-(aminomethyl)-1-(4,4,4-trifluorobutyl)benzimidazol-2-yl]methyl]-3-cyclopropyl-4H-quinazolin-2-one) is (S)-1-(2-fluoro-phenyl)-3-(2-oxo-5-phenyl-2,3-dihydro-1H-benzo[e][1,4]diazepin-3-yl)-urea, also known as RSV604.

Examples of suitable F-protein targeting anti-bodies include palivizumab and motavizumab.

Examples of suitable anti-inflammatory compounds include steroids (for example budesonide or fluticasone), non-steroids such as leukotriene antagonists, phosphodiesterase 4 inhibitors or TNF alpha inhibitors, and interleukin 8 or interleukin 9 inhibitors.

FIGS. 1A to 26A each show characterising data for compounds of the invention, as follows:

FIG. 1A: X-Ray Powder Diffraction Pattern for 1-[[5-(aminomethyl)-1-isopentyl-benzimidazol-2-yl]methyl]-3-cyclopropyl-4H-quinazolin-2-one monohydrate when measured using CuKa radiation.

FIG. 1B: DSC Thermogram for 1-[[5-(aminomethyl)-1-isopentyl-benzimidazol-2-yl]methyl]-3-cyclopropyl-4H-quinazolin-2-one monohydrate

FIG. 2: X-Ray Powder Diffraction Pattern for 1-[[5-(aminomethyl)-1-isopentyl-benzimidazol-2-yl]methyl]-3-cyclopropyl-4H-quinazolin-2-one dihydrate when measured using CuKa radiation.

FIG. 3A: X-Ray Powder Diffraction Pattern for 1-[[5-(aminomethyl)-1-isopentyl-benzimidazol-2-yl]methyl]-3-cyclopropyl-4H-quinazolin-2-one Form B when measured using CuKa radiation.

FIG. 3B: DSC Thermogram for 1-[[5-(aminomethyl)-1-isopentyl-benzimidazol-2-yl]methyl]-3-cyclopropyl-4H-quinazolin-2-one Form B

FIG. 4A: X-Ray Powder Diffraction Pattern for 1-[[5-(aminomethyl)-1-isopentyl-benzimidazol-2-yl]methyl]-3-cyclopropyl-4H-quinazolin-2-one Form C when measured using CuKa radiation.

FIG. 4B: DSC Thermogram for 1-[[5-(aminomethyl)-1-isopentyl-benzimidazol-2-yl]methyl]-3-cyclopropyl-4H-quinazolin-2-one Form C

FIG. 5A: X-Ray Powder Diffraction Pattern for 1-[[5-(aminomethyl)-1-isopentyl-benzimidazol-2-yl]methyl]-3-cyclopropyl-4H-quinazolin-2-one Form D when measured using CuKa radiation.

FIG. 5B: DSC Thermogram for 1-[[5-(aminomethyl)-1-isopentyl-benzimidazol-2-yl]methyl]-3-cyclopropyl-4H-quinazolin-2-one Form D

FIG. 6A: X-Ray Powder Diffraction Pattern for 1-[[5-(aminomethyl)-1-isopentyl-benzimidazol-2-yl]methyl]-3-cyclopropyl-4H-quinazolin-2-one Form E when measured using CuKa radiation.

FIG. 6B: DSC Thermogram for 1-[[5-(aminomethyl)-1-isopentyl-benzimidazol-2-yl]methyl]-3-cyclopropyl-4H-quinazolin-2-one Form E

FIG. 7A: X-Ray Powder Diffraction Pattern for 1-[[5-(aminomethyl)-1-isopentyl-benzimidazol-2-yl]methyl]-3-cyclopropyl-4H-quinazolin-2-one Form F when measured using CuKa radiation.

FIG. 7B: DSC Thermogram for 1-[[5-(aminomethyl)-1-isopentyl-benzimidazol-2-yl]methyl]-3-cyclopropyl-4H-quinazolin-2-one Form F

FIG. 8A: X-Ray Powder Diffraction Pattern for 1-[[5-(aminomethyl)-1-isopentyl-benzimidazol-2-yl]methyl]-3-cyclopropyl-4H-quinazolin-2-one Form G when measured using CuKa radiation.

FIG. 8B: DSC Thermogram for 1-[[5-(aminomethyl)-1-isopentyl-benzimidazol-2-yl]methyl]-3-cyclopropyl-4H-quinazolin-2-one Form G

FIG. 9A: X-Ray Powder Diffraction Pattern for 1-[[5-(aminomethyl)-1-isopentyl-benzimidazol-2-yl]methyl]-3-cyclopropyl-4H-quinazolin-2-one Form H when measured using CuKa radiation.

FIG. 9B: DSC Thermogram for 1-[[5-(aminomethyl)-1-isopentyl-benzimidazol-2-yl]methyl]-3-cyclopropyl-4H-quinazolin-2-one Form H

FIG. 10: X-Ray Powder Diffraction Pattern for 1-[[5-(aminomethyl)-1-isopentyl-benzimidazol-2-yl]methyl]-3-cyclopropyl-4H-quinazolin-2-one acetate salt, Form A, when measured using CuKa radiation.

FIG. 11: X-Ray Powder Diffraction Pattern for 1-[[5-(aminomethyl)-1-isopentyl-benzimidazol-2-yl]methyl]-3-cyclopropyl-4H-quinazolin-2-one acetate salt, Form B, when measured using CuKa radiation.

FIG. 12: X-Ray Powder Diffraction Pattern for 1-[[5-(aminomethyl)-1-isopentyl-benzimidazol-2-yl]methyl]-3-cyclopropyl-4H-quinazolin-2-one adipate salt, Form A, when measured using CuKa radiation.

FIG. 13: X-Ray Powder Diffraction Pattern for 1-[[5-(aminomethyl)-1-isopentyl-benzimidazol-2-yl]methyl]-3-cyclopropyl-4H-quinazolin-2-one adipate salt, Form B, when measured using CuKa radiation.

FIG. 14: X-Ray Powder Diffraction Pattern for 1-[[5-(aminomethyl)-1-isopentyl-benzimidazol-2-yl]methyl]-3-cyclopropyl-4H-quinazolin-2-one adipate salt, Form C, when measured using CuKa radiation.

FIG. 15: X-Ray Powder Diffraction Pattern for 1-[[5-(aminomethyl)-1-isopentyl-benzimidazol-2-yl]methyl]-3-cyclopropyl-4H-quinazolin-2-one cinnamate salt, Form A, when measured using CuKa radiation.

FIG. 16: X-Ray Powder Diffraction Pattern for 1-[[5-(aminomethyl)-1-isopentyl-benzimidazol-2-yl]methyl]-3-cyclopropyl-4H-quinazolin-2-one cinnamate salt, Form B, when measured using CuKa radiation.

FIG. 17: X-Ray Powder Diffraction Pattern for 1-[[5-(aminomethyl)-1-isopentyl-benzimidazol-2-yl]methyl]-3-cyclopropyl-4H-quinazolin-2-one stearate salt, Form A, when measured using CuKa radiation.

FIG. 18: X-Ray Powder Diffraction Pattern for 1-[[5-(aminomethyl)-1-isopentyl-benzimidazol-2-yl]methyl]-3-cyclopropyl-4H-quinazolin-2-one stearate salt, Form B, when measured using CuKa radiation.

FIG. 19: X-Ray Powder Diffraction Pattern for 1-[[5-(aminomethyl)-1-isopentyl-benzimidazol-2-yl]methyl]-3-cyclopropyl-4H-quinazolin-2-one stearate salt, Form C, when measured using CuKa radiation.

FIG. 20: X-Ray Powder Diffraction Pattern for 1-[[5-(aminomethyl)-1-isopentyl-benzimidazol-2-yl]methyl]-3-cyclopropyl-4H-quinazolin-2-one stearate salt, Form D, when measured using CuKa radiation.

FIG. 21A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one Form A when measured using CuKa radiation.

FIG. 21B: DSC Thermogram for 1-[[5-(aminomethyl)-1-(4,4,4-trifluorobutyl)benzimidazol-2-yl]methyl]-3-cyclopropyl-4H-quinazolin-2-one Form A.

FIG. 22A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one Form B when measured using CuKa radiation.

FIG. 22B: DSC Thermogram for 1-[[5-(aminomethyl)-1-(4,4,4-trifluorobutyl)benzimidazol-2-yl]methyl]-3-cyclopropyl-4H-quinazolin-2-one Form B.

FIG. 23A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one Form C when measured using CuKa radiation.

FIG. 23B: DSC Thermogram for 1-[[5-(aminomethyl)-1-(4,4,4-trifluorobutyl)benzimidazol-2-yl]methyl]-3-cyclopropyl-4H-quinazolin-2-one Form C.

FIG. 24A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one IPA solvate when measured using CuKa radiation.

FIG. 24B: DSC Thermogram for 1-[[5-(aminomethyl)-1-(4,4,4-trifluorobutyl)benzimidazol-2-yl]methyl]-3-cyclopropyl-4H-quinazolin-2-one IPA solvate.

FIG. 25A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one acetonitrile solvate when measured using CuKa radiation.

FIG. 25B: DSC Thermogram for 1-[[5-(aminomethyl)-1-(4,4,4-trifluorobutyl)benzimidazol-2-yl]methyl]-3-cyclopropyl-4H-quinazolin-2-one acetonitrile solvate.

FIG. 26A: X-Ray Powder Diffraction Pattern for 1-[[5-(aminomethyl)-1-(4,4,4-trifluorobutyl)benzimidazol-2-yl]methyl]-3-cyclopropyl-4H-quinazolin-2-one methanol solvate when measured using CuKa radiation.

FIG. 26B: DSC Thermogram for 1-[[5-(aminomethyl)-1-(4,4,4-trifluorobutyl)benzimidazol-2-yl]methyl]-3-cyclopropyl-4H-quinazolin-2-one methanol solvate.

The present invention will now be further explained by reference to the following illustrative examples.

General Methods

Unless otherwise stated, ¹H NMR spectra were recorded on a Bruker Avance spectrometer at 250 MHz and chemical shifts are reported in ppm relative to DMSO-d₆ or CDCl₃. Thin-layer chromatography was performed on pre-coated silica gel F-254 plastic plates (0.2 mm, Macherey-Nagel) and was visualized with UV light.

LC/MS spectra were recorded using one of two systems:

System 1:

Liquid Chromatograph: Agilent 1200 series, with PDA detector, scan range 190-400 nm. Mass spectrometer: Agilent MSD 6120 operating in electrospray ionisation mode with +ve/−ve ion switching. LC Conditions: Mobile phase A—0.1% formic acid/10 mM ammonium formate in water; Mobile phase B—acetonitrile.

Gradient:

| Time (mins.) | % B |
|---|---|
| 0 | 5 |
| 4 | 95 |
| 4.9 | 95 |
| 5 | 5 |

Flow rate: 1.0 ml/min. Column: Varian Pursuit Ultra 3 C18 50 mm×2.1 mm. Column temp: 50° C.

System 2:

Liquid Chromatograph: Waters Acquity UPLC, with PDA detector, (scan range 190-400 nm) and ELSD. Mass spectrometer: Waters SQD operating in electrospray ionisation mode with +ve/−ve ion switching. LC Conditions: Mobile phase A—0.1% formic acid in water; Mobile phase B—0.1% formic acid in acetonitrile.

### Gradient:

| Time (mins.) | % B |
|---|---|
| 1 | 5 |
| 0.2 | 5 |
| 4.5 | 95 |
| 6 | 95 |

Flow rate: 0.6 ml/min. Column: Waters Acquity UPLC BEH C18 50 mm×2.1 mm Column temp: 50° C.

High-performance liquid chromatography (HPLC) was carried out using the following system. Liquid Chromatograph: Waters 600 pump, W2700 Sample Manager, W996 PDA detector. Mass spectrometer: Waters ZQ operating in electrospray ionisation mode. LC Conditions: Mobile phase A—0.1% formic acid in water; Mobile phase B—0.1% formic acid in acetonitrile.

Gradient:

| Time (mins.) | % B |
|---|---|
| 2 | 5 |
| 15 | 75 |
| 16 | 100 |
| 18 | 100 |

Flow rate: 20 ml/min. Column: Gemini C18 50 mm×21.2 mm 5 μm 110A Axia (Phenomenex Ltd).

X-Ray Powder Diffraction

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits
Analytical Instrument: Bruker D4.

The X-ray powder diffraction spectra were determined by mounting a sample of the crystalline material on a Bruker single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5418 angstroms. The collimated X-ray source was passed through an automatic variable divergence slit set at V20 and the reflected radiation directed through a 5.89 mm is antiscatter slit and a 9.55 mm detector slit. The sample was exposed for 0.03 seconds per 0.00570° 2-theta increment (continuous scan mode) over the range 2 degrees to 40 degrees 2-theta in theta-theta mode. The running time was 3 minutes and 36 seconds. The instrument was equipped with a Position sensitive detector (Lynxeye). Control and data capture was by means of a Dell Optiplex 686 NT 4.0 Workstation operating with Diffract+software. Persons skilled in the art of X-ray powder diffraction will realise that the relative intensity of peaks can be affected by, for example, grains above 30 microns in size and non-unitary aspect ratios that may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values.

Differential Scanning Calorimetry (DSC)

Analytical Instrument: TA Instruments Q1000 DSC.

Typically less than 5 mg of material contained in a standard aluminium pan fitted with a lid was heated over the temperature range 25° C. to 300° C. at a constant heating rate of 10° C. per minute. A purge gas using nitrogen was used—flow rate 100 ml per minute.

All chemicals were purchased from commercial suppliers and used directly without further purification.

The abbreviations or terms used in the Examples have the following meanings:
AcOH: acetic acid
Boc: tert-butoxycarbonyl
c.: concentrated
CDI: N,N′-carbonyldiimidazole
cPr: cyclopropyl
DMF: N,N-dimethylformamide
DMSO: dimethylsulfoxide
EtOH: ethanol
EtOAc: ethylacetate
eq: molar equivalent
h: hour(s)
LiAlH₄: lithium aluminium hydride
Me: methyl
MeCN: acetonitrile
MeOH: methanol
min: minute(s)
NBS: N-Bromosuccinimide
Piv: pivaloyl
TFA: trifluoroacetic acid
THF: tetrahydrofuran
IPA: isopropylalcohol
MTBE: methyl tert-butyl ether
DIPEA: N,N-diisopropylethylamine
MsCl: methanesulfonyl chloride
TGA: thermogravimetric analysis

### EXAMPLES 1 to 4

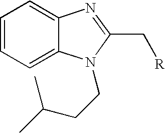

| Ex. | R | ¹H NMR | LC/MS |
|---|---|---|---|
| 1 | 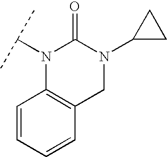 | (250 MHz, DMSO-d₆): δ0.58 (m, 2H), 0.75 (m, 2H), 0.97 (d, 6H), 1.62 (m, 2H), 1.75 (m, 1H), 2.61 (m, 1H), 4.25 (t, 2H), 4.39 (s, 2H), 5.32 (s, 2H), 6.93 (m, 1H), 7.12 (m, 3H), 7.15 (m, 2H), 7.51 (t, 2H) | 389 (MH⁺) |
| 2 | 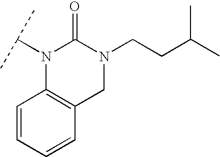 | (250 MHz, DMSO-d₆): δ0.86 (d, 12H), 1.61 (m, 6H), 4.25 (m, 4H), 4.90 (m, 4H), 7.20 (m, 1H), 7.26 (m, 2H), 7.31 (m, 1H), 7.54 (m, 2H), 7.62 (m, 2H) | 419 (MH⁺) |
| 3 | 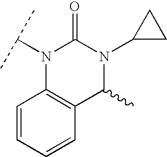 | (250 MHz, DMSO-d₆): δ0.55 (m, 2H), 0.68 (m, 1H), 0.84 (m, 1H), 0.98 (dd, 6H), 1.37 (d, 3H), 1.64 (m, 2H), 1.71 (m, 1H), 2.67 (m, 1H), 4.32 (m, 2H), 4.55 (q, 1H), 5.34 (dd, 2H), 6.95 (m, 1H), 7.11 (m, 3H), 7.15 (m, 2H), 7.49 (m, 2H) | 404 (MH⁺) |
| 4 | 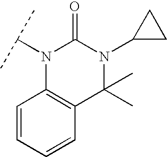 | (250 MHz, DMSO-d₆): δ0.46 (m, 2H), 0.81 (m, 2H), 0.98 (d, 6H), 1.66 (s, 6H), 2.36 (m, 1H), 3.34 (s, 2H), 4.33 (t, 2H), 5.36 (s, 2H), 6.99 (m, 1H), 7.13 (m, 3H), 7.21 (m, 1H), 7.31 (m, 1H), 7.46 (m, 1H), 7.51 (m, 1H) | 418 (MH⁺) |

Examples 1 to 4 were prepared by reacting the 2-chloromethylbenzimidazole derivative with the indicated quinazolinone compound using a procedure analogous to that described in WO 01/95910 (page 85).

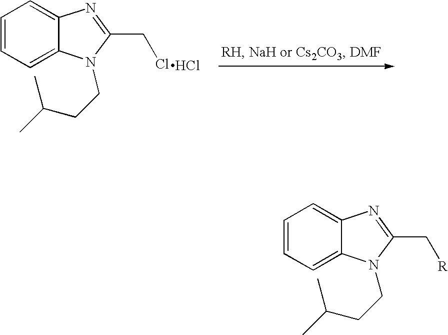

Starting materials for Examples 1 to 4:

### 2-(Chloromethyl)-1-isopentyl-benzimidazole, HCl salt

Prepared using the procedure described in WO 01/95910 (page 21).

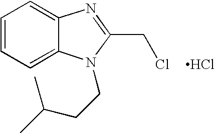

### 3-Cyclopropyl-1,4-dihydroquinazolin-2-one

Prepared using a method analogous to that described by Coyne et al., J. Med. Chem. 1968, 11, 1208:

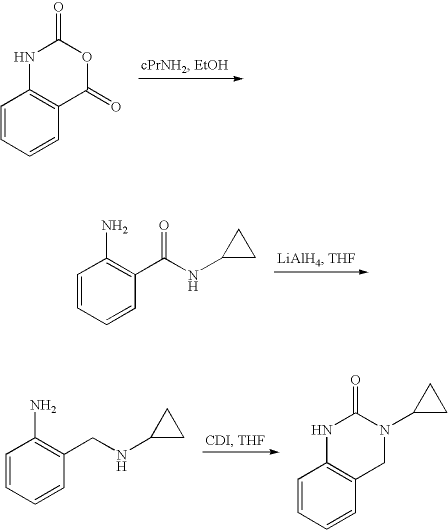

### 2-Amino-N-cyclopropylbenzamide

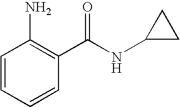

To a stirred solution of isatoic anhydride (167 g, 1.02 mol, 1 eq) in EtOH (1 L) was added triethylamine (142 mL, 1.02 mol, 1 eq). Cyclopropylamine (71 mL, 1.02 mol, 1 eq) was added drop wise at such a rate that the temperature did not rise above 30° C. Once the addition was complete the reaction was heated to 70° C. for 16 h. The reaction was allowed to cool to room temperature, resulting in the formation of a precipitate. This was collected by filtration and washed with diethyl ether (500 mL). The solid was then slurried in diethyl ether (1.5 L) for 1 h, filtered and washed with diethyl ether to give of the title compound (25.5 g). The filtrate from the first precipitation was evaporated and slurried in diethyl ether (1 L) for 1 h, filtered and washed with diethyl ether (500 mL) to give further is title compound (12.0 g). Again the filtrate was evaporated down and slurried in diethyl ether (500 mL), filtered and washed with diethyl ether (250 mL) to give further title compound (6.9 g, 44.2 g in total, 25%).

¹H NMR (250 MHz, DMSO-d₆): δ0.35 (m, 2H), 0.46 (m, 2H), 2.59 (m, 1H), 6.09 (br.s, 2H), 6.26 (m, 1H), 6.46 (m, 1H), 6.89 (m, 1H), 7.20 (dd, 1H), 7.82 (br.s, 1H). LC/MS 177 (MH⁺).

### 2-[(Cyclopropylamino)methyl]aniline

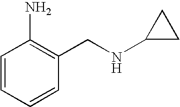

To a stirred 1 M solution of LiAlH₄ in THF (85 mL, 85 mmol, 2.7 eq), cooled in an ice bath, was added a solution of 2-amino-N-cyclopropylbenzamide (5 g, 31 mmol, 1 eq) in THF (70 mL) at such a rate that the reaction temperature did not rise above 10° C. Once the addition was complete the reaction was heated at 60° C. for 16 h. The reaction was cooled to 0° C. and a 1:1 mixture of water:THF (25 mL) was added at such rate that the temperature of the reaction did not rise above 30° C. A 1 M aqueous solution of NaOH (40 mL) was added cautiously. The reaction was filtered and washed with THF (2×25 mL). To the filtrate was added EtOAc (150 mL) and water (50 mL). The aqueous layer was separated and extracted with EtOAc (2×50 mL). The combined organic extracts were dried (Na₂SO₄), filtered and the solvent evaporated in vacuo to afford the title compound as a crude brown solid (4.6 g).

¹H NMR (250 MHz, DMSO-d₆): δ0.03 (m, 1H), 0.11 (m, 1H), 1.22 (m, 1H), 1.82 (m, 3.38 (d, 2H), 4.72 (br.s, 2H), 4.88 (br.s, 1H), 6.24 (m, 1H), 6.35 (d, 1H), 6.69 (m, 2H). LC/MS 163 (MH⁺).

### 3-Cyclopropyl-1,4-dihydroquinazolin-2-one

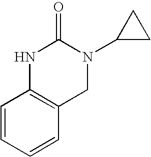

To a stirred solution of 2-[(cyclopropylamino)methyl]aniline (60 g, 0.37 mol, 1 eq) in THF (600 mL) at room temperature, under an atmosphere of nitrogen, was added CDI (90 g, 0.55 mol, 1.5 eq) portion wise. Once the addition was complete the reaction was heated at 60° C. for 16 h. The reaction was allowed to cool to room temperature and then concentrated in vacuo to afford a brown oil. The oil was dissolved in CH₂Cl₂ (750 mL) and washed with water (750 mL). The water was separated and back extracted with CH₂Cl₂ (2×250 mL). The combined organic layers were washed with a 1 M aqueous solution of HCl (750 mL), brine (750 mL), dried (MgSO₄), filtered and concentrated in vacuo to give an off-white solid. The solid was purified by flash column chromatography eluting with 1:1 EtOAc:heptane to give the title compound as a white solid (23.1 g, 33%).

¹H NMR (250 MHz, DMSO-d₆): δ0.33 (m, 2H), 0.48 (m, 2H), 2.32 (m, 1H), 4.09 (s, 2H), 6.53 (m, 1H), 6.61 (m, 1H), 6.87 (m, 2H), 8.88 (br.s, 1H). LC/MS 189 (MH⁺).

### 3-Cyclopropyl-4-methyl-1,4-dihydroquinazolin-2-one

Prepared using an analogous method to that described by Hasegawa et al., Biorg. Med. Chem., 2005, 13, 3721 to prepare 4-methyl-1,4-dihydroquinazolin-2-ones.

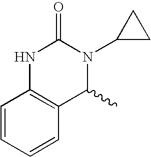

¹H NMR (250MHz, DMSO-d₆): δ0.28 (m, 2H), 0.41 (m, 1H), 0.62 (m, 1H), 1.03 (d, 3H). 2.35 (m, 1H), 4.25 (q, 1H), 6.52 (m, 1H), 6.62 (m, 1H), 6.86 (m, 1H), 9.08 (br.s, 1H). LC/MS 203 (MH⁺).

### 3-Cyclopropyl-4,4-dimethyl-1H-quinazolin-2-one

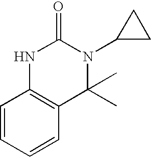

A mixture of 3-cyclopropyl-4-methyl-4-(trichloromethyl)-1H-quinazolin-2-one (5 g, 15.6 mmol, 1 eq), triethylamine (1.31 mL, 93.6 mmol, 6 eq) and 10% palladium on carbon (1 g) in MeOH (30 mL) was stirred at room temperature under an atmosphere of hydrogen. After 48 h, the reaction mixture was filtered through a pad of Celite. The filtrate was then concentrated in vacuo to dryness. The residue was purified by column chromatography using 500:8:1 through to 200:8:1 of CH₂Cl₂:EtOH:NH₃ to give the title compound as a clear oil that formed a pale yellow solid on standing (982 mg, 29%).

¹H NMR (250 MHz, DMSO-d₆): δ0.55 (2H, m), 0.82 (2H, m), 1.55 (s, 6H), 2.19 (m, 1H), 6.80 (m, 1H), 6.89 (m, 1H), 7.12 (m, 1H), 7.22 (m, 1H), 9.46 (br.s, 1H). LC/MS 217 (MH⁺).

### EXAMPLES 5 to 23

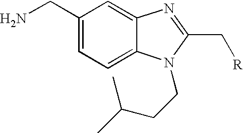

| Ex. | R | ¹H NMR | LC/MS |
|---|---|---|---|
| 5 | 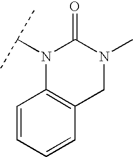 | (250 MHz, DMSO-d₆): δ0.99 (d, 6H), 1.71 (br.s, 3H), 2.95 (s, 3H), 4.19 (d, 2H), 4.50 (m 2H), 4.55 (s, 2H), 5.59 (s, 2H), 7.07 (m, 2H), 7.22 (m, 2H), 7.68 (d, 2H), 7.91 (d, 2H), 8.62 (br.s, 2H) | 392 (MH⁺) |
| 6 | 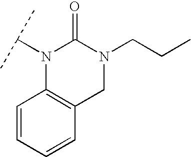 | (250 MHz, DMSO-d₆): δ0.88 (t, 3H), 0.95 (d, 6H), 1.51-1.68 (m, 5H), 3.33 (t, 2H), 3.95 (s, 2H), 4.26 (t, 2H), 4.46 (s, 2H), 5.33 (s, 2H), 6.94 (m, 1H), 7.10-7.16 (m, 3H), 7.25 (dd, 1H), 7.48 (d, 1H), 7.57 (d, 1H), 8.39 (s, 1H) | 419 (MH⁺) |
| 7 | 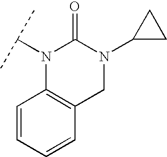 | (250 MHz, DMSO-d₆): δ0.61 (m, 2H), 0.76 (m, 2H), 0.99 (d, 6H), 1.72 (br.s, 3H), 2.63 (m, 1H), 4.19 (d, 2H), 4.51 (s, 4H), 5.58 (s, 2H), 7.07 (m, 2H), 7.27 (m, 2H), 7.70 (d, 1H), 7.93 (m, 2H), 8.64 (br.s, 3H) | 418 (MH⁺) |
| 8 | 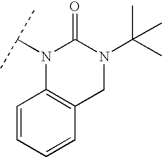 | (250 MHz, DMSO-d₆): δ0.0.95 (d, 6H), 1.43 (s, 9H), 1.59 (m, 2H), 1.68 (m, 1H), 4.01 (s, 2H), 4.26 (t, 2H), 4.35 (s, 2H), 5.32 (s, 2H), 6.94 (m, 1H), 7.10 (m, 2H), 7.20 (m, 1H), 7.28 (m, 2H), 7.51 (d, 1H), 7.61 (s, 1H). | 434 (MH⁺) |
| 9 | 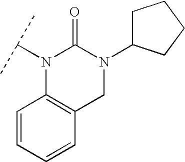 | (250 MHz, DMSO-d₆): δ0.94 (d, 6H), 1.48-1.55 (m, 5H), 1.58-1.71 (m, 6H), 3.78 (s, 2H), 4.23 (t, 2H), 4.32 (s, 2H), 4.72 (m, 1H), 5.32 (s, 2H), 6.93 (m, 1H), 7.10-7.26 (m, 5H), 7.36-7.43 (m, 2H), 7.48 (m, 1H). | 446 (MH⁺) |
| 10 | 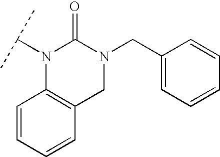 | (250 MHz, DMSO-d₆): δ0.97 (d, 6H), 1.71 (br.s, 3H), 4.17 (d, 2H), 4.61 (s, 2H), 5.63 (s, 2H), 7.05 (m, 2H), 7.20 (m, 7H), 7.68 (d, 1H), 7.93 (s, 2H), 8.60 (br.s, 3H) | 468 (MH⁺) |
| 11 | 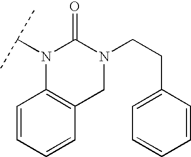 | (250 MHz, DMSO-d₆): δ0.98 (d, 6H), 1.60 (d, 3H), 2.90 (t, 2H), 3.57 (m, 2H), 3.75 (s, 2H), 4.25 (t, 2H), 4.48 (s, 2H), 5.35 (s, 2H), 6.94 (m, 1H), 7.28 (m, 8H), 7.38 (d, 1H), 7.48 (s, 1H) | 482 (MH⁺) |
| 12 | 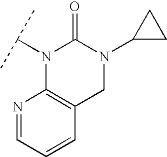 | (250 MHz, DMSO-d₆): δ 0.65 (m, 2H), 0.77 (m, 2H), 0.97 (d, 6H), 1.68 (m, 3H), 2.69 (m, 1H), 3.75 (s, 2H), 4.30 (s, 2H), 5.42 (s, 2H), 6.97 (m, 1H), 7.16 (1 dd, H), 7.40 (m, 2H), 7.63 (dd, 1H), 8.06 (dd, 1H) | 419 (MH⁺) |
| 13 | 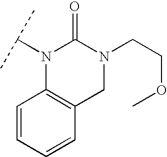 | (250 MHz, DMSO-d₆): δ0.98 (d, 6H), 1.71 (m, 3H), 3.26 (s, 3H), 3.54 (m, 5H), 4.18 (d, 2H), 4.47 (m, 2H), 4.62 (s, 2H), 5.57 (s, 2H), 7.08 (m, 2H), 7.23 (m, 2H), 7.66 (d, 1H), 7.90 (m, 2H), 8.60 (br.s, 3H) | 434 (MH⁺) |
| 14 | 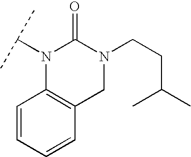 | (250 MHz, DMSO-d₆): δ0.93 (m, 14H), 1.54 (m, 6H), 3.40 (m, 2H), 3.75 (s, 2H), 4.23 (t, 2H), 4.45 (s, 2H), 5.33 (s, 2H), 6.94 (m, 1H), 7.15 (m, 4H), 7.36 (d, 1H), 7.47 (s, 1H) | 448 (MH⁺) |
| 15 | 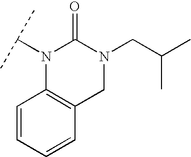 | (250 MHz, DMSO-d₆): δ0.90 (d, 6H), 0.97 (d, 6H), 1.59 (m, 4H), 2.00 (m, 2H), 3.75 (s, 2H), 4.23 (t, 2H), 4.45 (s, 2H), 5.34 (s, 2H), 6.94 (m, 1H), 7.19 (m, 4H), 7.37 (d, 1H), 7.47 (s, 1H) | 434 (MH⁺) |
| 16 | 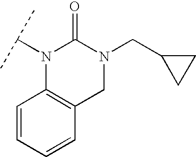 | (250 MHz, DMSO-d₆): δ0.29 (m, 2H), 0.51 (m, 2H), 1.59 (m, 4H), 1.97 (br.s, 1H), 3.30 (d, 3H), 3.75 (s, 2H), 4.25 (t, 2H), 4.55 (s, 2H), 5.35 (s, 2H), 6.98 (m, 1H), 7.15 (m, 4H), 7.37 (d, 1H), 7.48 (s, 1H) | 432 (MH⁺) |
| 17 | 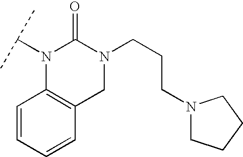 | (250 MHz, DMSO-d₆): δ0.97 (d, 6H), 1.67 (m, 11H), 2.40 (m, 4H), 3.37 (m, 4H), 3.76 (s, 2H), 4.23 (t, 2H), 4.48 (s, 2H), 5.34 (s, 2H), 6.93 (m, 1H), 7.15 (m, 4H), 7.37 (d, 1H), 7.48 (s, 1H) | 489 (MH⁺) |
| 18 | 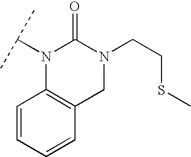 | (250 MHz, DMSO-d₆): δ0.97 (d, 6H), 1.59 (m, 3H), 2.11 (s, 3H), 2.73 (t, 2H), 3.26 (br.s, 2H), 3.57 (t, 2H), 3.76 (s, 2H), 4.25 (t, 2H), 4.54 (s, 2H), 5.34 (s, 2H), 6.95 (m, 1H), 7.15 (m, 4H), 7.38 (d, 1H), 7.48 (s, 1H) | 452 (MH⁺) |
| 19 | 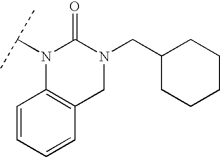 | (250 MHz, DMSO-d₆): δ0.97 (d, 6H), 1.18 (m, 3H), 1.64 (m, 9H), 3.27 (m, 4H), 3.75 (s, 2H), 4.23 (t, 2H), 4.45 (s, 2H), 5.35 (s, 2H), 6.94 (m, 1H), 7.19 (m, 4H), 7.37 (d, 1H), 7.48 (s, 1H) | 474 (MH⁺) |
| 20 | 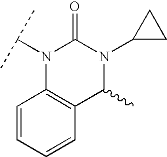 | (250 MHz, DMSO-d₆): δ0.55 (m, 2H), 0.69 (m, 1H), 0.83 (m, 1H), 0/97 (dd, 6H), 1.37 (d, 3H), 1.70 (m, 3H), 2.66 (m, 1H), 3.74 (m, 1H), 4.30 (m, 2H), 4.54 (q, 1H), 5.34 (dd, 2H), 6.94 (m, 1H), 7.15 (m, 4H), 7.39 (m, 2H) | 433 (MH⁺) |
| 21 | 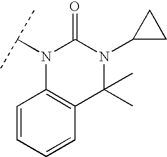 | (250 MHz, DMSO-d₆): δ0.45 (m, 2H), 0.82 (m, 2H), 0.98 (d, 6H), 1.63 (m, 9H), 2.36 (m, 1H), 3.97 (s, 2H), 4.32 (m, 2H), 5.37 (s, 2H), 7.04 (m, 2H), 7.16 (m, 1H), 7.28 (m, 2H), 7.51 (m, 2H), 8.36 (br.s) | 447 (MH⁺) |
| 22 | 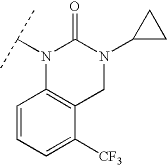 | (250 MHz, DMSO-d₆): δ0.53 (m, 2H), 0.77 (m, 2H), 0.98 (d, 6H), 1.72 (m, 3H), 2.65 (m, 1H), 4.15 (m, 2H), 4.47 (t, 2H), 4.60 (s, 2H), 5.56 (s, 2H), 7.42-7.46 (m, 3H), 7.61 (m, 1H), 7.86 (m, 2H) | 486 (MH⁺) |
| 23 | 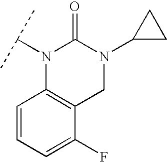 | (250 MHz, DMSO-d₆): δ0.74 (m, 2H), 0.92 (m, 2H), 0.96 (m, 6H), 1.63 (m, 3H), 2.66 (m, 1H), 4.18 (m, 2H), 4.45 (t, 2H), 4.53 (s, 2H), 5.56 (s, 2H), 6.92 (m, 2H), 7.26 (m, 1H), 7.64 (m, 1H), 7.89-7.94 (m, 2H) | 436 (MH⁺) |

Examples 5 to 23 were prepared using a procedure analogous to that described in WO 03/053344 (page 20; Scheme XIII).

The process involved coupling of an appropriately protected aminomethyl derivative with the indicated quinazolinone followed by deprotection:

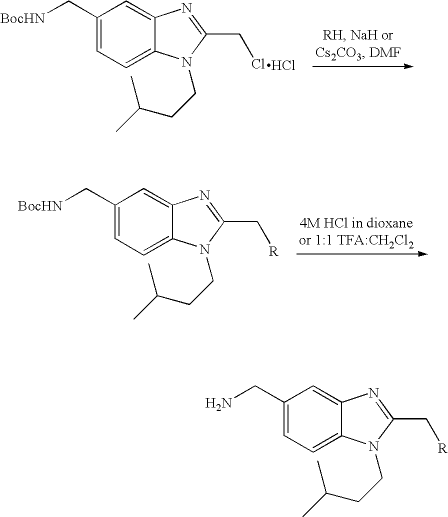

In some cases, the compounds were also prepared by reduction of the corresponding cyano derivative:

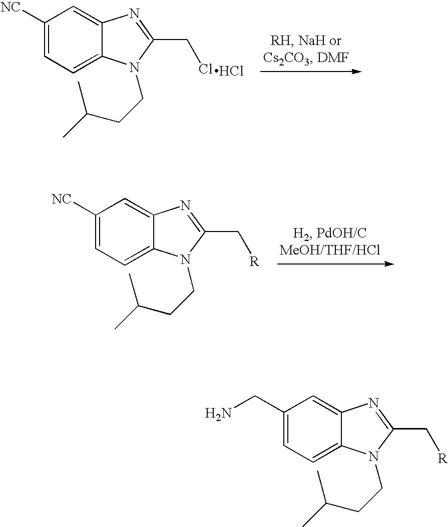

Typical Reaction of a Benzimidazole with a Quinazolinone:

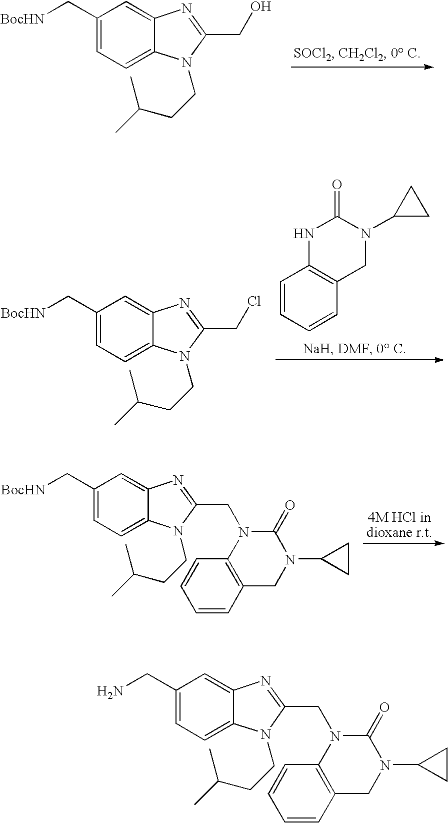

### tert-Butyl N-[[2-[(3-cyclopropyl-2-oxo-4H-quinazolin-1-yl)methyl]-1-isopentyl-benzimidazol-5-yl]methyl]carbamate

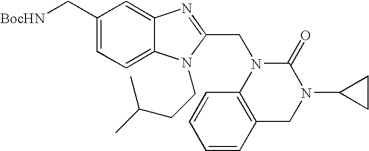

To as stirred solution of tent-butyl N-[[2-(hydroxymethyl)-1-isopentyl-benzimidazol-5-yl]methyl]carbamate (1 g, 2.88 mmol, 1 eq) in CH₂Cl₂ (10 mL) at 0° C. was added thiony chloride (420 μL, 5.76 mmol, 2 eq) drop wise via syringe. The reaction was then allowed to warm to room temperature. After 30 min the reaction was concentrated in vacuo to dryness affording an off-white solid. The solid was dissolved in DMF (4 mL) and added to a stirred suspension of 3-cyclopropyl-1,4-dihydroquinazolin-2-one (542 mg, 2.88 mmol, 1 is eq) and a 60% dispersion of NaH in mineral oil (346 mg, 8.64 mmol, 3 eq) in DMF (6 mL), under an atmosphere of nitrogen. The reaction was then stirred at room temperature for 16 h. The reaction was poured onto water (100 mL) and the resulting suspension was filtered. The residue was recrystallised from boiling EtOH (4 mL) to give the title compound as a pale yellow solid (831 mg, 57%).

¹H NMR (250 MHz, DMSO-d₆): δ059 (m, 2H), 0.73 (m, 2H), 0.96 (d, 6H), 1.36 (s, 9H), 1.58 (m, 2H), 1.66 (m, 1H), 2.61 (m, 1H), 4.15 (d, 2H), 4.25 (t, 2H), 4.39 (s, 2H), 5.32 (s, 2H), 6.93 (m, 1H) 7.12-7.20 (m, 4H), 7.34-7.43 (m, 3H). LC/MS 519 (MH⁺).

### 1-[[5-(Aminomethyl)-1-isopentyl-benzimidazol-2-yl]methyl]-3-cyclopropyl-4H-quinazolin-2-one (Example 7)

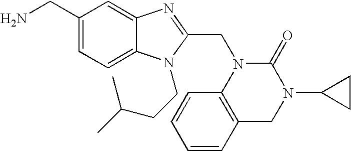

To a stirred solution of tert-butyl N-[[2-[(3-cyclopropyl-2-oxo-4H-quinazolin-1-yl)methyl]-1-isopentyl-benzimidazol-5-yl]methyl]carbamate (830 mg, 1.60 mmol, 1 eq) in CH₂Cl₂ (10 mL) at room temperature was added a 4 M solution of HCl in dioxane (5 mL). After 2 h the reaction was concentrated in vacuo to dryness. The residue was dissolved in is water (10 mL) and basified with a saturated aqueous solution of NaHCO₃ (8 mL). This mixture was extracted with CH₂Cl₂ (3×8 mL) and the combined extracts dried (MgSO₄), filtered and concentrated in vacuo to dryness to give the title compound as white solid (471 mg, 71%).

¹H NMR (250 MHz, DMSO-d₆): δ0.61 (m, 2H), 0.76 (m, 2H), 0.99 (d, 6H), 1.72 (br.s, 3H), 2.63 (m, 1H), 4.19 (d, 2H), 4.51 (s, 4H), 5.58 (s, 2H), 7.07 (m, 2H), 7.27 (m, 2H), 7.70 (d, 1H), 7.93 (m, 2H), 8.64 (br.s, 3H). LC/MS 418 (MH⁺).

Starting materials for Examples 5 to 23:

Benzimidazole Formation:

Prepared by the following route:

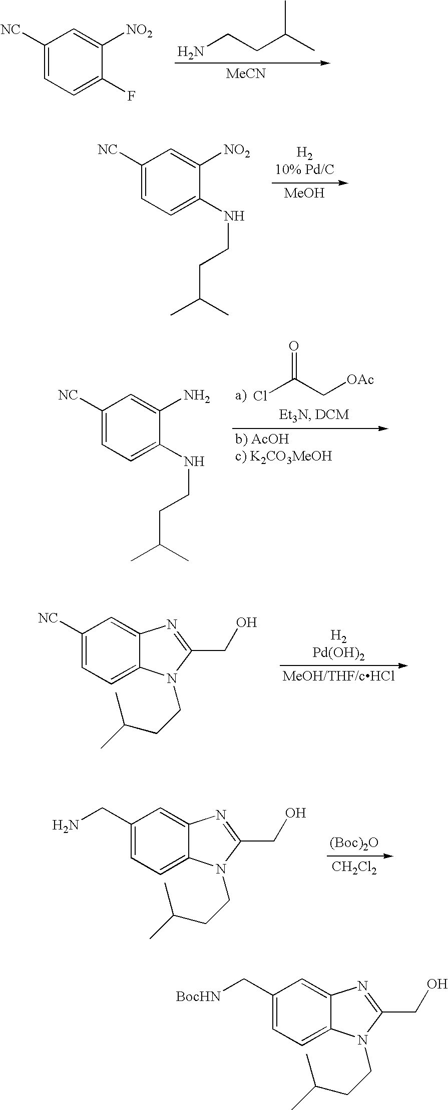

### 4-(Isopentylamino)-3-nitro-benzonitrile

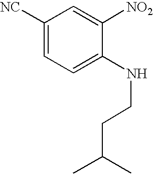

To a stirred solution of 4-fluoro-3-nitrobenzonitrile (110 g, 0.663 mol, 1 eq) in acetonitrile (1100 mL), under an atmosphere of nitrogen, was added isoamylamine (153.5 mL, 1.32 mol, 2 eq) drop wise over 20 minutes with cooling (ice/water bath). The temperature was kept below 30° C. The mixture was then stirred at room temperature for 1 h. The mixture was poured onto water (2000 mL) and the resulting solid was collected by filtration, washed with water (2×500 mL) and pulled dry. The solid was dried overnight in vacuo to give the title compound as a yellow solid (149.7 g, 97%).

¹H NMR (250 MHz, DMSO-d₆) δ0.93 (d, 6H), 1.53 (q, 2H), 1.68 (m, 1H), 3.44 (q, 2H), 7.18 (d, 1H), 7.83 (dd, 1H), 8.50 (m, 1H), 8.53 (m, 1H). LC/MS 234 (MH⁺).

### 3-Amino-4-(isopentylamino)benzonitrile

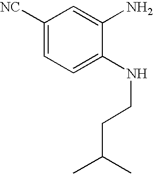

To a mixture of 10% Pd/C (60% wet, 82.2 g, 30.7 mmol Pd) in MeOH (1150 mL), under an atmosphere of nitrogen, was added 4-(isopentylamino)-3-nitro-benzonitrile (143 g, 0.614 mol). Hydrogen gas was bubbled through the mixture for 6 h with stirring. The mixture was filtered through Celite. The Celite was washed with MeOH (2×400 mL) and the combined filtrate/washings were concentrated in vacuo to give a wet red solid. The solid was dissolved in CH₂Cl₂ (700 mL) and EtOAc (300 mL) and the resulting solution dried over MgSO₄ and concentrated in vacuo to give the title compound as a dark red solid (121 g, 97%).

¹H NMR (250 MHz, DMSO-d₆) δ0.94 (d, 6H), 1.51 (q, 2H), 1.72 (m, 1H), 3.11 (q, 2H), 4.96 (br.s, 2H), 5.27 (br.t, 1H), 6.47 (d, 1H), 6.78 (d, 1H), 6.93 dd, 1H). LC/MS 204 (MH⁺).

### 2-(Hydroxymethyl)-1-isopentyl-benzimidazole-5-carbonitrile

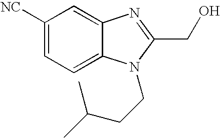

To a stirred solution of 3-amino-4-(isopentylamino)benzonitrile (127.8 g, 0.63 mol, 1 eq) in CH₂Cl₂ (1900 mL), under an atmosphere of nitrogen, was added triethylamine (175 mL, 1.26 mol, 1 eq). Acetoxyacetyl chloride (71.1 mL, 0.66 mol, 1.05 eq) was added drop wise with cooling (ice/water bath) keeping the temperature below 25° C. The mixture was then stirred at room temperature for 2 h. The mixture was concentrated in vacuo and the resulting solid suspended in AcOH (640 mL). The mixture was stirred at 80° C. overnight. Excess AcOH was removed in vacuo and the crude residue dissolved in MeOH (1.5 L). K₂CO₃ (437 g, 3.17 mol, 5 eq) was added and the mixture stirred at room temperature overnight. The reaction mixture was filtered and the filtrate concentrated in vacuo to dryness. The resulting solid was slurried in water (2000 mL) for 30 min then filtered, washed with water (2×500 mL) and pulled dry to give the title compound as a pale brown solid (144 g, 94% yield over 3 steps). This material was used directly in the next stage without further purification.

¹H NMR (250 MHz, DMSO-d₆) δ 0.97 (d, 6H), 1.67 (m, 3H), 4.35 (t, 2H), 4.77 (d, 2H), 5.76 (t, 1H), 7.67 (dd, 1H), 7.77 (dd, 1H), 8.16 (d, 1H). LC/MS 244 (MH⁺).

### [5-(Aminomethyl)-1-isopentyl-benzimidazol-2-yl]methanol

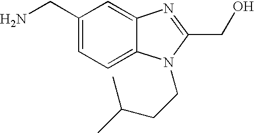

A mixture of 20% Pd(OH)₂/C (50% wet, 28.9 g) and MeOH (5350 mL), THF (2700 mL) and c.HCl (168 mL) was stirred at room temperature and 2-(hydroxymethyl)-1-isopentyl-benzimidazole-5-carbonitrile (144 g, 0.59 mol) was added. Hydrogen gas was bubbled is through the mixture for 2 h then the mixture was left to stir under a blanket of hydrogen overnight. The reaction mixture was purged with nitrogen and the mixture concentrated in vacuo. The residue was dissolved in water (250 mL) and basified to pH 9 with a saturated aqueous solution of Na₂CO₃ (2000 mL). The mixture was extracted with EtOAc (3×500 mL) and the aqueous fraction left to stand overnight. A precipitate formed in the aqueous fraction and this was collected by filtration, the aqueous filtrate was then concentrated in vacuo to dryness. The solids were combined and slurried in EtOH (2000 mL) for 30 min and filtered. The filtrate was then concentrated in vacuo to dryness to give the title compound as a pale brown solid (144.4 g, 97%).

¹H NMR (250 MHz, DMSO-d₆) δ 0.96 (d, 6H), 1.65 (m, 3H), 3.84 (s, 2H), 4.27 (t, 2H), 4.71 (s, 2H), 7.24 (dd, 1H), 7.45 (d, 1H), 7.51 (d, 1H). LC/MS 248 (MH⁻).

### tert-Butyl N-[[2-(hydroxymethyl)-1-isopentyl-benzimidazol-5-yl]methyl]-carbamate

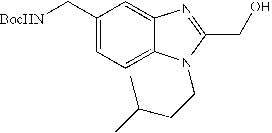

To a stirred suspension of [5-(aminomethyl)-1-isopentyl-benzimidazol-2-yl]methanol (141.4 g, 0.572 mol) in CH₂Cl₂ (3000 mL), under an atmosphere of nitrogen, was added diisopropylethylamine (198 mL, 1.144 mol, 2 eq). A solution of di-tert-butyl dicarbonate (124.8 g, 0.572 mol, 1 eq) in CH₂Cl₂ (1000 mL) was added drop wise over 30 min and the mixture stirred at room temperature. The mixture was filtered and the filtrate washed with water (1000 mL). The aqueous fraction was extracted with CH₂Cl₂ (1000 mL). The combined CH₂Cl₂ layers were washed with brine (1000 mL), dried (MgSO₄) and filtered. The organics were concentrated in vacuo. The resulting solid was slurried in EtOAc (200 mL) and heptanes (800 mL) for 30 min then filtered and washed with heptane (500 mL). The solid were suspended in CH₂Cl₂ (300 mL) and loaded onto a pad of silica (1 kg). The silica was eluted with 50% EtOAc/heptane (5×2000 mL) followed by 70% is EtOAc/heptanes (2×2000 mL). The second batch of 70% EtOAc/heptanes was found to contain only product with no by-product. The silica was eluted with 10% MeOH/EtOAc (10,000 mL) to flush the product from the silica. All batches of eluent containing only clean product were then concentrated in vacuo to dryness to give the title compound as a white solid (90.5 g, 46%).

¹H NMR (250 MHz, DMSO-d₆) δ 0.96 (d, 6H), 1.41 (s, 9H), 1.65 (m, 3H), 4.22 (d, 2H), 4.27 (t, 2H), 4.71 (d, 2H), 7.14 (d, 1H), 7.41-7.46 (m, 3H). LC/MS 348 (MH⁺).

### tert-Butyl N-[[2-(chloromethyl)-1-isopentyl-benzimidazol-5-yl]methyl]carbamate, HCl salt

Prepared using the procedure described in WO 03/053344 (page 82).

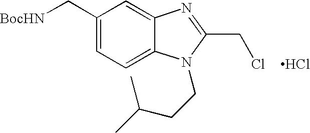

### 2-(Chloromethyl)-1-isopentyl-benzimidazole-5-carbonitrile, HCl salt

Prepared using the procedure described in WO 03/053344 (page 27).

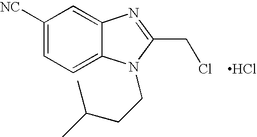

Quinazolinone Formation:

3-Substituted-1,4-dihydroquinazolin-2-ones were prepared using a method analogous to that described by Coyne et al., J. Med. Chem. 1968, 11, 1208 except for those of Examples 12, 22, and 23

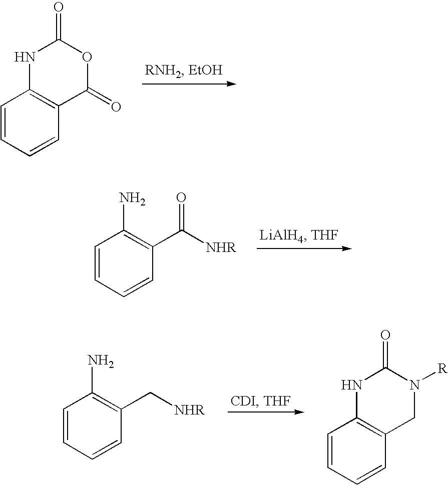

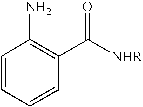

| R | ¹H NMR | LC/MS |
|---|---|---|
| 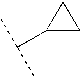 | (250 MHz, DMSO-d₆): δ 0.35(m, 2H), 0.46(m, 2H), 2.59(m, 1H), 6.09(br.s, 2H), 6.26(m, 1H), 6.46(m, 1H), 6.89(m, 1H), 7.20(dd, 1H), 7.82 (br.s, 1H) | 177 (MH⁺) |
| 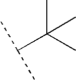 | | 193 (MH⁺) |
| 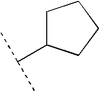 | (250 MHz, DMSO-d₆): δ 1.54(m, 4H), 1.69(m, 2H), 1.86(m, 2H), 4.19(m, 1H), 6.32(s, 2H), 6.52 (m, 1H), 6.68(m, 1H), 7.12(m, 1H), 7.46(m, 1H), 8.02(d, 1H) | 205 (MH⁺) |
| 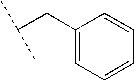 | (250 MHz, DMSO-d₆): δ 3.96(d, 2H), 5.95(br.s, 2H), 6.04(m, 1H), 6.24(dd, 1H), 6.66(m, 1H), 6.77(m, 1H), 6.81(m, 5H), 7.08(dd, 1H), 8.30(t, 1H) | 227 (MH⁺) |
| 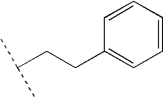 | | 241 (MH⁺) |
| 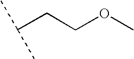 | (250 MHz, DMSO-d₆): δ 3.29(s, 3H), 3.43(m, 4H), 6.33(br.s, 2H), 6.52(m, 1H), 6.75(dd, 1H), 7.13 (m, 1H), 7.49(dd, 1H), 8.11(br.s, 1H) | 195 (MH⁺) |
| 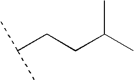 | | 207 (MH⁺) |
| 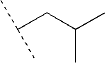 | | 193 (MH⁺) |
| 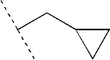 | | 191 (MH⁺) |
| 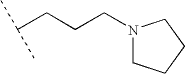 | | 248 (MH⁺) |
| 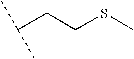 | | 211 (MH⁺) |
| 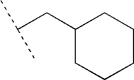 | | 233 (MH⁺) |

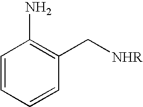

| R | ¹H NMR | LC/MS |
|---|---|---|
| 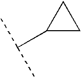 | (250 MHz, DMSO-d6): δ 0.03(m, 1H), 0.11(m, 1H), 1.22(m, 1H), 1.82(m, 1H), 3.38(d, 2H), 4.72(br.s, 2H), 4.88(br.s, 1H), 6.24(m, 1H), 6.35 (d, 1H), 6.69(m, 2H) | 163 (MH⁺) |
| 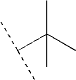 | (250 MHz, DMSO-d₆): δ 1.05(s, 9H), 1.87(br.s, 1H), 3.88(s, 2H), 7.48(m, 1H), 7.66(m, 1H), 7.73(m, 1H), 7.85(dd, 1H) | 179 (MH⁺) |
| 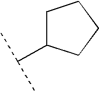 | | 191 (MH⁺) |
| 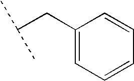 | (250 MHz, DMSO-d₆): δ 2.33(br.s, 1H), 3.61(s, 2H), 3.66(s, 2H), 5.12(br.s, 2H), 6.49(m, 1H), 6.62(dd, 1), 6.94(t, 2H), 7.22(m, 6H) | 213 (MH⁺) |
| 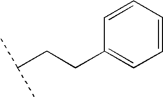 | | 227 (MH⁺) |
| 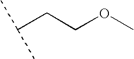 | (250 MHz, DMSO-d₆): δ 3.23(s, 3H), 3.39(t, 4H), 3.62(s, 2H), 4.24(br.s, 1H), 5.09(br.s, 1H), 6.47 (m, 1H), 6.60(d, 1H), 6.92(m, 2H) | 181 (MH⁺) |
| 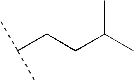 | | 193 (MH⁺) |
| 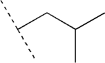 | | 179 (MH⁺) |
| 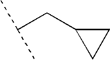 | | 177 (MH⁺) |
| 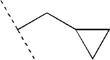 | | 177 (MH⁺) |
| 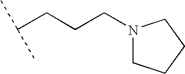 | | 234 (MH⁺) |
| 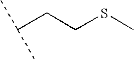 | | 197 (MH⁺) |
| 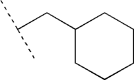 | | 219 (MH⁺) |

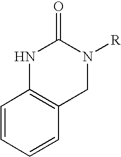

| R | ¹H NMR | LC/MS |
|---|---|---|
| 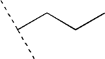 | (250 MHz, DMSO-d₆): δ 0.82(t, 3H), 1.53(m, 2H), 3.24(t, 2H), 4.38(s, 2H), 6.75(m, 1H), 6.84(m, 1H), 7.05(m, 2H), 9.11(br.s, 1H) | 190 (MH⁺) |
| 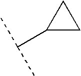 | (250 MHz, DMSO-d₆): δ 0.33(m, 2H), 0.48(m, 2H), 2.32(m, 1H), 4.09(s, 2H), 6.53(m, 1H), 6.61 (m, 1H), 6.87(m, 2H), 8.88(br.s, 1H) | 189 (MH⁺) |
| 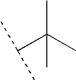 | (250 MHz, DMSO-d₆): δ 1.43(s, 9H), 4.33(s, 2H), 6.76(m, 1H), 6.84(m, 1H), 7.12(m, 2H), 9.05 (br.s, 1H) | 205 (MH⁺) |
| 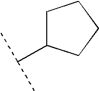 | (250 MHz, DMSO-d₆): δ 1.55-1.67(m, 8H), 4.29(s, 2H), 4.73(m, 1H), 6.78(d, 1H), 6.87(m, 1H), 7.13 (m, 2H), 9.17(br.s, 1H) | 217 (MH⁺) |
| 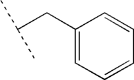 | (250 MHz, DMSO-d₆): δ 4.40(s, 2H), 4.54(s, 2H), 6.82(t, 2H), 7.03(m, 1H), 7.10(m, 1H), 7.29(m, 5H), 9.20(br.s, 1H) | 239 (MH⁺) |
| 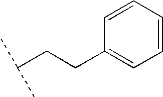 | | 253 (MH⁺) |
| 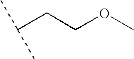 | (250 MHz, DMSO-d₆): δ 3.26(s, 3H), 3.48(m, 4H), 4.46(s, 2H), 6.77(d, 1H), 6.84(m, 1H), 7.06(d, 1H), 7.11(d, 1H), 9.09(br.s, 1H) | 207 (MH⁺) |
| 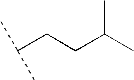 | | 219 (MH⁺) |
| 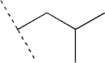 | | 205 (MH⁺) |
| 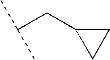 | | 203 (MH⁺) |
| 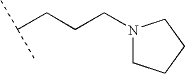 | | 260 (MH⁺) |
| 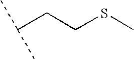 | | 223 (MH⁺) |
| 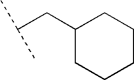 | | 245 (MH⁺) |

### 3-Cyclopropyl-1,4-dihydropyrido[2,3-d]pyrimidin-2-one

Prepared according to the following scheme:

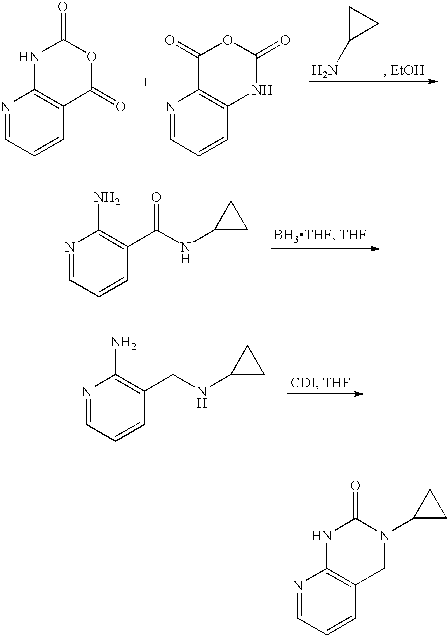

### 1H-Pyrido[2,3-d][1,3]oxazine-2,4-dione and 1H-pyrido[3,2-d][1,3]oxazine-2,4-dione

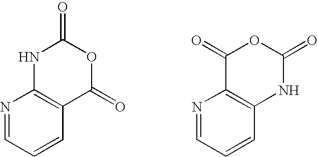

The title compounds were synthesised according to the procedure described in Le Count, D. J.; Dewsbury, D. J. Synthesis 1982, 11, 972-973. A 9:1 mixture of 1H-pyrido[2,3-d][1,3]oxazine-2,4-dione and 1H-pyrido[3,2-d][1,3]oxazine-2,4-dione was obtained as an off-white solid (6.14 g, 56%).

¹H NMR (250 MHz, DMSO-d₆) δ7.31 (dd, 1H), 8.31 (dd, 1H), 8.65 (dd, 1H), 12.28 (br.s, 1H) for 1H-pyrido[2,3-d][1,3]oxazine-2,4-dione; 87.54 (dd, 1H), 7.71 (dd, 1H), 8.51 (dd, 1H), 11.78 (br.s, 1H) for 1H-pyrido[3,2-d][1,3]oxazine-2,4-dione. LC/MS 163 (M−H).

### 2-Aminopyridine-3-carboxamide

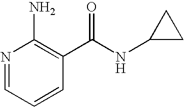

To a stirred 9:1 mixture of 1H-pyrido[2,3-d][1,3]oxazine-2,4-dione and 1H-pyrido[3,2-d][1,3]oxazine-2,4-dione (6 g, 37 mmol, 1 eq) in EtOH at room temperature was added cyclopropylamine (2.54 g, 44 mmol, 1.2 eq). The reaction was heated at 50° C. After 30 min the reaction was allowed to cool to room temperature and then concentrated in vacuo to dryness. The reside was purified by flash column chromatography, eluting with initially 400:8:1 CH₂Cl₂:EtOH:NH₃ and then 200:8:1 CH₂Cl₂:EtOH:NH₃, to give the title compound as a white solid (5.13 g, 78%). R_{f}=0.36 (200:8:1 CH₂Cl₂:EtOH:NH₃).

¹H NMR (250 MHz, DMSO-d₆) δ0.52 (m, 2H), 0.65 (m, 2H), 2.79 (m, 1H), 6.53 (dd, 1H), 7.05 (br.s, 2H), 7.81 (dd, 1H), 8.01 (dd, 1H), 8.35 (br.d, 1H). LC/MS 178 (MH⁺).

### 3-[(Cyclopropylamino)methyl]pyridin-2-amine

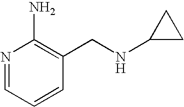

To a stirred suspension of 2-aminopyridine-3-carboxamide (4 g, 22.6 mmol, 1 eq) in THF (40 mL) at 0° C., under nitrogen, was added a 1 M solution of BH₃THF in THF (67.8 mL, 67.8 mmol, 3 eq) drop wise via a dropping funnel. Once the addition was complete the reaction was allowed to warm to room temperature and then heated at reflux. After 2 h the reaction was allowed to cool to room temperature and then further cooled in an ice-water bath. MeOH was added cautiously until no further effervescence was seen. The reaction was concentrated in vacuo to give a pale yellow oil. The oil was filtered through a pad of silica, eluting with 100:8:1 CH₂Cl₂:EtOH:NH₃. The filtrate was concentrated in vacuo to provide the title compound as a pale yellow oil (1.85 g, 50%).

¹H NMR (250 MHz, DMSO-d₆) δ0.0 (m, 2H), 0.08 (m, 2H), 1.76 (m, 1H), 2.55 (br.s, 1H), 3.46 (s, 2H), 6.37 (t, 1H), 6.55 (br.s, 2H), 7.35 (d, 1H), 7.65 (d, 1H). LC/MS no mass ion detected.

### 3-Cyclopropyl-1,4-dihydropyrido[2,3-d]pyrimidin-2-one

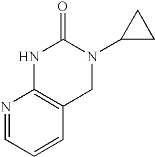

A stirred mixture of 3-[(cyclopropylamino)methyl]pyridin-2-amine (1.85 g, 15 mmol, 1 eq) and N,N′-carbonyldiimidazole (7.31 g, 45 mmol, 3 eq) in THF (30 mL) was heated at reflux. After 16 h the reaction was allowed to cool to room temperature and concentrated in vacuo to dryness to afford a yellow oil. The oil was purified by flash column chromatography eluting with 200:8:1 CH₂Cl₂:EtOH:NH₃ to give a tacky off-white solid. Trituration with EtOH yielded the title compound as a white solid (890 mg, 41%). R_{f}=0.23 (200:8:1 CH₂Cl₂:EtOH:NH₃).

¹H NMR (250 MHz, DMSO-d₆): δ0.33 (m, 2H), 0.46 (m, 2H), 2.30 (m, 1H), 4.11 (s, 2H), 6.64 (dd, 1H), 7.26 (dd, 1H), 7.78 (dd, 1H), 9.39 (br.s, 1H). LC/MS 190 (MH⁺).

### 3-Cyclopropyl-5-fluoro-1,4-dihydroquinazolin-2-one

Prepared according to the following scheme:

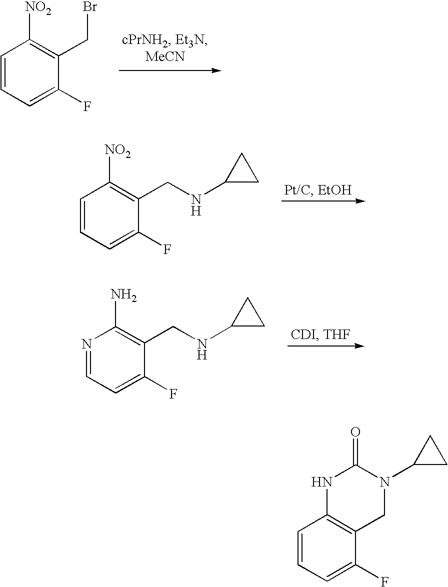

### N-[(2-Fluoro-6-nitro-phenyl)methyl]cyclopropanamine

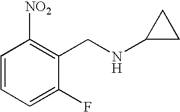

To a stirred solution of cyclopropylamine (1.47 mL, 1.71 mmol, 2 eq) and triethylamine (2.95 mL, 1.71 mmol, 2 eq) in MeCN (20 mL) at 0° C. was added a solution of 2-fluoro-6-nitrobenzyl bromide (2 g, 8.54 mmol, 1 eq) in MeCN (10 mL) drop wise via syringe. Once the addition was complete the reaction was allowed to warm to room temperature. After 16 h the reaction was filtered and the filtrate concentrated in vacuo to dryness. The residue was purified by flash column chromatography, eluting with 100% CH₂Cl₂, to give the title to compound as an off-white solid.

¹H NMR (250 MHz, CDCl₃): δ0.0.30 (m, 2H), 0.35 (m, 2H), 1.99 (m, 1H), 2.20 (br.s, 1H), 3.99 (d, 2H), 7.25-7.34 (m, 2H), 7.62 (m, 1H). LC/MS 211 (MH⁺)

### 2-[(Cyclopropylamino)methyl]-3-fluoro-aniline

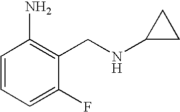

A solution of N-[(2-fluoro-6-nitro-phenyl)methyl]cyclopropanamine (1.3 g, 6.18 mmol, 1 eq) in EtOH (130 mL) was hydrogenated using H-cube apparatus fitted with a 10% Pt/C cartridge. The reaction was concentrated in vacuo to dryness to afford the title compound as an off-white solid (1.12g, 100%).

¹H NMR (250 MHz, CDCl₃): δ0.22 (m, 2H), 0.33 (m, 2H), 1.54 (br.s, 1H), 2.10 (m, 1H), 3.77 (d, 2H), 4.48 (br.s, 2H), 6.28 (m, 2H), 6.84 (m, 1H). LC/MS 180 (MH⁺)

### 3-Cyclopropyl-5-fluoro-1,4-dihydroquinazolin-2-one

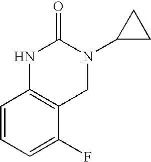

To a stirred solution of 2-[(cyclopropylamino)methyl]-3-fluoro-aniline (960 mg, 5.33 mmol, 1 eq) in THF (50 mL) at room temperature was added CDI (1.04 g, 6.41 mmol, 1.2 eq) drop wise via syringe. After heating at reflux for 16 h the reaction was allowed to cool to room temperature and then concentrated in vacuo to dryness. The residue was dissolved in CH₂Cl₂ (20 mL) and washed with water (3×15 mL). The organic fraction was dried (MgSO₄), filtered and concentrated in vacuo to afford the title compound as an off-white solid.

¹H NMR (250 MHz, CDCl₃): δ0.76 (m, 2H), 0.94 (m, 2H), 2.67 (m, 1H), 4.50 (s, 2H), 6.46 (m, 1H), 6.68 (m, 1H), 716 (m, 1H). LC/MS 206 (MH⁻)

### 3-cyclopropyl-5-(trifluoromethyl)-1,4-dihydroquinazolin-2-one

Prepared according to the following scheme:

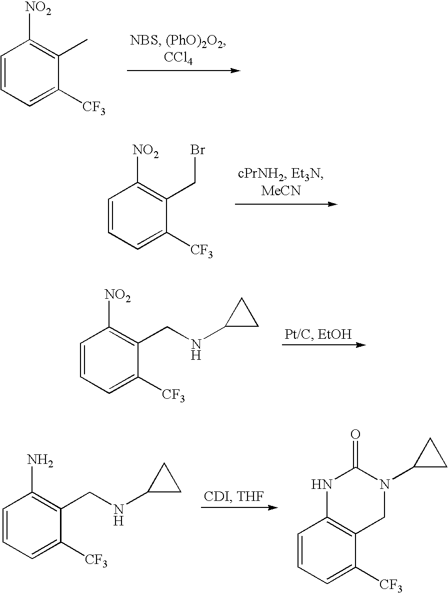

### 2-(Bromomethyl)-1-nitro-3-(trifluoromethyl)benzene

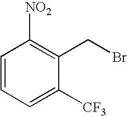

To a stirred solution of NBS (1.2 g, 6.78 mmol, 1.1 eq) and benzoyl peroxide (146 mg, 0.616 mmol, 0.1 eq) in CCl₄ (15 mL), under nitrogen, at room temperature was added a solution of 2-methyl-3-nitrobenzonitrile (1 g, 6.17 mmol, 1 eq) via syringe. The reaction was then heated at reflux. After 16 h the reaction was allowed to cool to room temperature s and filtered. The filtrate was concentrated in vacuo to dryness to give the title compound as a pale yellow oil.

¹H NMR (250 MHz, CDCl₃): δ4.84 (s, 2H), 7.85 (m, 1H), 8.16 (m, 1H), 8.32 (m, 1H). LC/MS no mass ion detected

### N-[[2-nitro-6-(trifluoromethyl)phenyl]methyl]cyclopropanamine

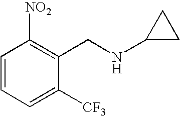

The title compound was prepared in an analogous manner to that described for N-[(2-fluoro-6-nitro-phenyl)methyl]cyclopropanamine.

¹H NMR (250 MHz, CDCl₃): δ0.29 (m, 2H), 0.37 (m, 2H), 2.14 (m, 1H), 4.21 (d, 2H), 7.54 (m, 1H), 7.85-7.90 (m, 2H LC/MS 261 (MH⁺)

### 2-[(Cyclopropylamino)methyl]-3-(trifluoromethyl)aniline

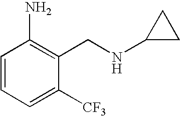

The title compound was prepared in an analogous manner to that described for 2-[(cyclopropylamino)methyl]-3-fluoro-aniline

¹H NMR (250 MHz, CDCl₃): δ0.34 (m, 2H), 0.46 (m, 2H), 2.20 (m, 1H), 3.91 (br.s, 2H), 6.82 (m, 1H), 6.98 (m, 1H), 7.10 (m, 1H). LC/MS 231 (MH⁻)

### 3-cyclopropyl-5-(trifluoromethyl)-1,4-dihydroquinazolin-2-one

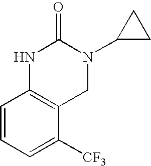

The title compound was prepared in an analogous manner to that described for 3-cyclopropyl-5-fluoro-1,4-dihydroquinazolin-2-one.

¹H NMR (250MHz, CDCl₃): δ0.63 (m, 2H), 0.85 (m, 2H), 2.59 (m, 1H), 4.49 (s, 2H), 6.88 (m, 1H), 7.19 (m, 2H), 7.95 (br.s, 1H). LC/MS 257 (MH⁺)

### EXAMPLE 7(I)

### Larger Scale Preparation of 1-[[5-(Aminomethyl)-1-isopentyl-benzimidazol-2-yl]methyl]-3-cyclopropyl-4H-quinazolin-2-one Monohydrate

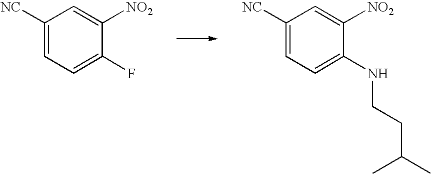

In a 50 L vessel under a nitrogen atmosphere were charged 4-fluoro-3-nitrobenzonitrile is (2880 g, 17.3 mol) and DMF (28.8 L). The contents were cooled to 5° C. before isopentylamine (4030 mL, 34.6 mol) was added dropwise over 70 minutes. The reaction mixture was allowed to warm to room temperature and stirred for 1 hour. The vessel contents were divided evenly into two portions and each portion was added to water (26.2 L). A solid subsequently precipitated from the solution and was filtered off. The filter cakes from each portion were combined and washed with water (2×13 L). The solid was re-charged into the 50 L vessel and slurried in 20 L water before being filtered and rinsed with heptane (20 L). The solids were dried under vacuum at 45° C. for 72 hours to provide 4-(isopentylamino)-3-nitro-benzonitrile (3944.8 g, 98% yield).

¹H NMR (300 MHz, CDCl₃) δH 0.98(d, 6H), 1.67(m, 3H), 3.34(m, 2H), 6.90(d, 1H), 7.58(dd, 1H), 8.36(br. S, 1H) and 8.48(d, 1H).

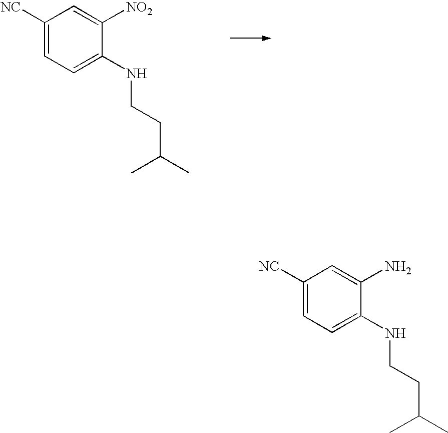

10% Pd/C (266 g, 0.15 mol) was charged to the reaction vessel followed by MeOH (13.4 L) and 4-(isopentylamino)-3-nitro-benzonitrile (1680 g, 7.2 mol). The reaction mixture was stirred vigorously under a H₂ atmosphere overnight, after which the catalyst was filtered off. The filtrate was concentrated under reduced pressure and once dry, MeOH (5 L) was added to the reaction flask and this was concentrated under reduced pressure. The resulting solid was collected and dried under vacuum at 40° C. to provide 3-amino-4-(isopentylamino)benzonitrile (1442.1 g, 99%) with a purity of >95% by ¹H NMR and 98.2% by HPLC.

¹H NMR (300MHz, CDCl₃) δH 0.96(d, 6H), 1.56(m, 2H), 1.73(m, 1H), 3.15(m, 2H), 3.27(br. s, 2H), 3.88(br. s, 1H), 6.60(d, 1H), 6.92(d, 1H) and 7.16(dd, 1H).

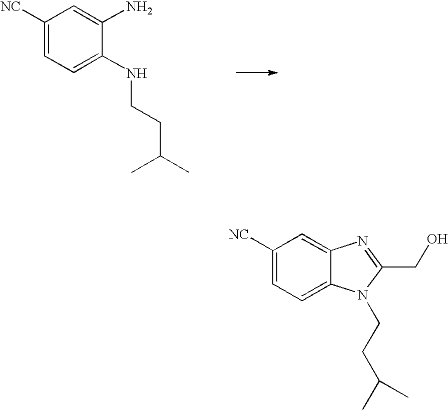

To a 50 L vessel were charged 3-amino-4-(isopentylamino)benzonitrile (2378 g, 11.7 mol), THF (24.5 L), and triethylamine (3.27 L, 23.4 mol). The solution was cooled to 0-5° C. before acetoxyacetyl chloride (1.32 L, 12.3 mol) in THF (11.55 L) was added dropwise, whilst maintaining the reaction temperature below 15° C. The reaction was then stirred at room temperature for 40 minutes. The suspension was filtered and the filtrate concentrated in vacuo. The resulting solid was re-suspended in acetic acid (12.3 L) and heated at 80° C. for 18 hours. The dark solution was concentrated under reduced pressure and toluene (3×2 L) was added to azeotrope the residual acetic acid from the oil. This crude oil was then added to a suspension of methanol (28 L) and potassium carbonate (6 kg). On complete consumption of starting material, water (28 L) was added to the suspension portion wise. The reaction was neutralised using acetic acid (1.4 L) before a second charge of water (28 L) was added. The suspension was filtered and the filter cake rinsed with water (10 L). The solid was dried in a vacuum oven at 40° C. to provide 2-(hydroxymethyl)-1-isopentyl-benzimidazole-5-carbonitrile (2471.7 g, 87%), with an HPLC purity of 98%,

¹H NMR (300 MHz, CDCl₃) δH 1.02(d, 6H), 1.71(m, 3H), 4.34 (m, 2H), 4.90(s, 2H), 5.12(br. s, 1H), 7.37(d,1H), 7.51(dd, 1H) and 7.98(s, 1H).

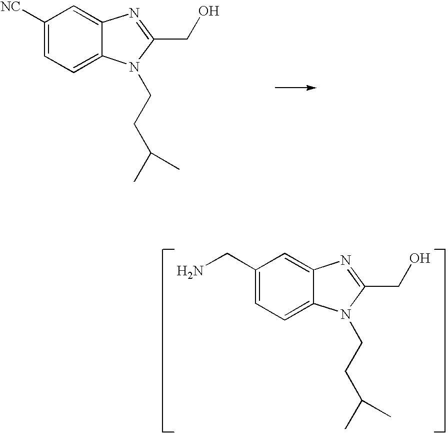

Wet Raney Ni (386 g) was allowed to settle before the water was decanted off. Methanol/ammonia (300 mL) was added to rinse residual water from the catalyst. The catalyst was subsequently charged to the vessel using methanol/ammonia (1 L) under a positive stream of nitrogen gas. 2-(hydroxymethyl)-1-isopentyl-benzimidazole-5-carbonitrile (800 g, 3.3 mol) was washed into the vessel with methanol/ammonia (14 L). The reaction was stirred at room temperature under a hydrogen atmosphere (5 bar) for 18 is hours. The catalyst was filtered off, to afford the crudeproduct, [5-(aminomethyl)-1-isopentyl-benzimidazol-2-yl]methanol, in solution, with an LC purity of 94%. This was concentrated to approximately half volume in vacuo, and used directly in the next stage of the preparation.

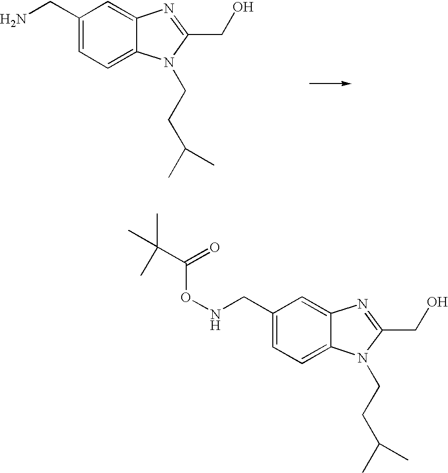

To a 50 L vessel was charged crude 5-(aminomethyl)-1-isopentyl-benzimidazol-2-yl]methanol (theoretically 2436 g, 9.9 mol) in methanol (18 L) A solution of di-tert-butyl dicarbonate (1930 g, 8.9 mol) in methanol (1.5 L) was added dropwise to the reaction mixture over 90 minutes whilst maintaining the reaction temperature below 20° C. The reaction was stirred at room temperature for 16 hours before the addition of 10% w/w aqueous sodium bisulfite (20 L). The suspension was allowed to stir for 16 hours before being filtered. The filter cake was washed with water (6 L) and pulled dry to afford the crude product, which was combined with two further batches and recrystallised from toluene (11.5 L). The resulting precipitate was washed with toluene (2×5.75 L) and heptane (3×5.75 L) then suction dried for 2 hours. The material was subjected to a further recrystallisation from toluene (10.5 L), similarly washing the filter cake with toluene (2×5.75 L.) and heptane (3×5.75 L). Following drying (vacuum oven at 45° C., 48 h), the product tent-butyl N-[[2-(hydroxymethyl)-1-isopentyl-benzimidazol-5-yl]methyl]-carbamate was obtained as a light brown solid (4353 g, 53%), with an LC purity of 98.22%.

¹H NMR (300 MHz, CDCl₃) δ 0.99 (d, 6H), 1.45 (s, 9H), 1.68 (m, 3H), 4.20 (t, 2H), 4.34 (d, 2H), 4.83 (s, 2H), 4.92(m, 1H), 5.41 (br. s, 1H), 7.20 (m, 2H), 7.49-7.46 (m, 1H).

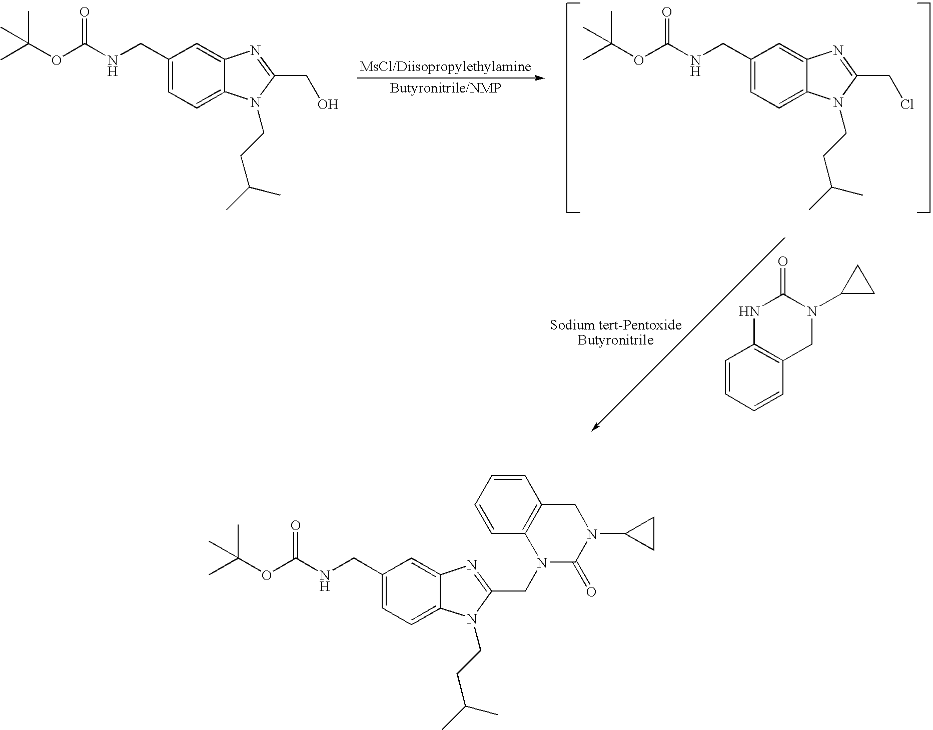

To a stirred suspension of tert-butyl {[2-(hydroxymethyl)-1-isopentyl-1H-benzimidazol-yl]methyl}carbamate (150 g, 431.53 mmol, 1.00 mol eq) in a mixture of butyronitrile (1.05 L) and N-methylpyrolidinone (150 ml) under an inert atmosphere was added diisopropylethylamine (135.5 ml, 776.76 mmol, 1.80 mol eq). The mixture was cooled to 0° C. and a solution of methanesulphonyl chloride (46.76 ml, 604.15 mmol, 1.40 mol eq) in butyronitrile (75 ml) was added at such a rate so as the temperature did not exceed 5° C. This was followed by a butyronitrile (75 ml) line wash. The resultant solution was allowed to warm to 25° C. and stirred for 3 hours to complete the conversion to the intermediate tert-butyl {[2-(chloromethyl)-1-isopentyl-1H-benzimidazol-5-yl]methyl}carbamate. A solution of 18.5% wt/vol aqueous citric acid solution (450 ml) was added and the contents were agitated for 15 minutes. The mixture was allowed to settle and the lower aqueous phase separated and discarded. A solution of 15% wt/vol potassium bicarbonate (450 ml) was added and the contents agitated for 15 minutes. The mixture was allowed to settle and the lower aqueous phase separated and discarded. Water (450 ml) was added and the is contents agitated for 15 minutes. The mixture was allowed to settle and the lower aqueous phase separated and discarded. Butyronitrile (375 ml) was added and the contents were distilled at 100 mmHg, collecting 900 ml of distillates. The remaining solution was held at 60° C. 3-Cyclopropyl-3,4-dihydroquinazolin-2(1H)-one (91.71 g, 453.11 mmol, 1.05 mol eq), sodium tert-pentoxide (52.53 g, 453.11 mmol, 1.05 mol eq) and butyronirile (712.5 ml) were charged to a second vessel and the contents placed under an inert atmosphere and warmed to 40° C. until a complete solution was obtained. The solution was then cooled to 25° C. This solution was added to the solution of tert-butyl {[2-(chloromethyl)-1-isopentyl-1H-benzimidazol-5-yl]methyl}carbamate in butyronitrile at 60° C. over 60 minutes followed by a 30 minute stir after the addition was complete. A solution of sodium tert-pentoxide (2.50 g, 21.58 mmol, 0.05 mol eq) in butyronitrile (37.5 ml) was then added and the contents stirred for 30 minutes to complete the conversion to the desired product, tert-butyl({2-[(3-cyclopropyl-2-oxo3,4-dihydroquinazolin-1(2H)-yl)methyl]-1-isopentyl-1H-bezimidazol-5-yl}methyl)carbamate. The reaction was quenched by adding a solution of glacial acetic acid (60 ml, 1046.5 mmol) in water (390 ml). The contents were taken to reflux and held at reflux for 15 minutes. The mixture was then cooled to 85° C. and allowed to settle for 15 minutes. The lower aqueous phase was separated and discarded. The contents were then cooled to 70° C. and seeded with tert-butyl({2-[(3-cyclopropyl-2-oxo3,4-dihydroquinazolin-1(2H)-yl)methyl]-1-isopentyl-1H-bezimidazol-5-yl}methyl)carbamate to initiate crystallisation. The mixture was held at 70° C. for 2 hours to fully establish crystallisation and then cooled over 6 hours to 25° C. Isohexane (450 ml) was added over 2 hours and the slurry stirred at 25° C. for a further 2 hours. The mixture was then filtered and washed twice with isohexane (300 ml). The product was dried in the vacuum oven at <200 mHg and 40° C. to constant weight.

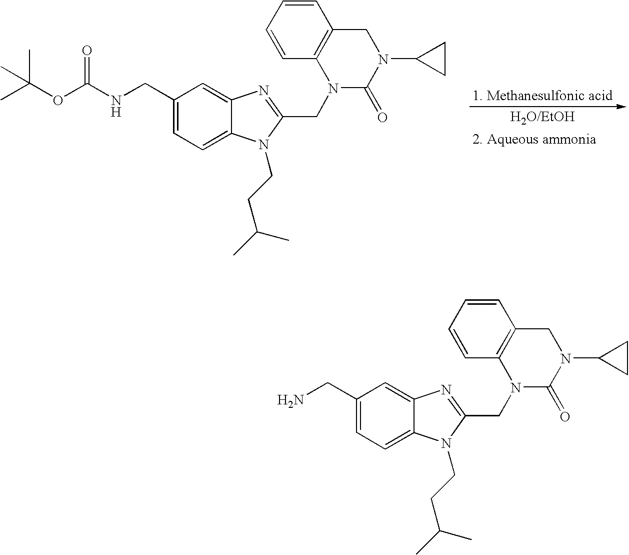

tert-Butyl N-[[2-[(3-cyclopropyl-2-oxo-4H-quinazolin-1-yl)methyl]-1-isopentyl-benzimidazol-5-yl]methyl]carbamate (150 g, 289.77 mmol, 1.0 mol eq) was suspended in a mixture of ethanol (675 ml) and water (375 ml) and the contents placed under an inert atmosphere. The slurry was warmed to 60° C. and a solution of methanesulphonic acid (47.5 ml, 724.42 mmol, 2.50 mol eq) added in such a way that the temperature did not exceed 65° C. Water (75 ml) was added as a line wash and the solution stirred at 60° C. for is 10 hours to complete the reaction. The solution was cooled to 20° C. and screened through a 1μ filter, followed by a line wash made up of ethanol (75 ml) and water (75 ml). The solution was warmed to 40° C. and aqueous ammoinia (197.83 ml, 1.74 mol, 6.0 mol eq) added followed by a water (75 ml) line wash. The contents were seeded with 1-[[5-(aminomethyl)-1-isopentyl-benzimidazol-2-yl]methyl]-3-cyclopropyl-4H-quinazolin-2-one and stirred at 40° C. for 2 hours to establish crystallization. The slurry was cooled to 20° C. over 2 hours and then water (300 ml) was added over a further 2 hours. The slurry was stirred for 2 hours at 20° C. and then filtered. The solid was washed twice with water (300 ml) and dried in the vacuum oven at 300 mbar with an air bleed to a water content of approximately 4% w/w.

¹H NMR (250 MHz, DMSO-d₆): δ0.61 (m, 2H), 0.76 (m, 2H), 0.99 (d, 6H), 1.72 (br.s, 3H), 2.63 (m, 1H), 4.19 (d, 2H), 4.51 (s, 4H), 5.58 (s, 2H), 7.07 (m, 2H), 7.27 (m, 2H), 7.70 (d, 1H), 7.93 (m, 2H), 8.64 (br.s, 3H).

TGA analysis indicated a weight loss of approximately 4.0%, suggesting a monohydrated form.

For this larger scale synthesis (except for the final product), ¹H NMR spectra were recorded on a Bruker AC3000 Series spectrometer at 300 MHz and chemical shifts are reported in ppm relative to CDCl₃.

HPLC method and LC conditions were as follows:

System: Agilent1100 series liquid chromatograph

Column: XBridge Phenyl 3.5 μm 4.6×150 mm

Mobile phase A: Acetonitrile: Water: Trifluoroacetic acid (5:95:0.1)

Mobile phase B: Acetonitrile: Water: Trifluoroacetic acid (95:5:0.1)

Flow rate: 1.0 ml.min⁻¹

Detection: UV at 210/246 nm

Column temperature: 30° C.

Gradient:

| Time (min) | % A | % B |
|---|---|---|
| 0 | 100 | 0 |
| 15 | 0 | 100 |
| 20 | 0 | 100 |
| 22 | 100 | 0 |

The product obtained in Example 7(i) was analysed by XRPD. The X-ray powder diffraction spectra showed the material to be crystalline.

1-[[5-(aminomethyl)-1-isopentyl-benzimidazol-2-yl]methyl]-3-cyclopropyl-4H-quinazolin-2-one monohydrate is characterised by providing an X-ray powder diffraction pattern, substantially as shown in FIG. 1A. The ten most prominent peaks are shown in Table 1:

TABLE 1
Ten most Prominent X-Ray Powder Diffraction peaks for
1-[[5-(aminomethyl)-1-isopentyl-benzimidazol-2-
yl]methyl]-3-cyclopropyl-4H-quinazolin-2-one monohydrate

| Angle 2- | | Relative |
|---|---|---|
| Theta (2θ) | Intensity % | Intensity |
| 8.157 | 100.0 | vs |
| 9.697 | 52.5 | vs |
| 16.342 | 41.3 | vs |
| 8.424 | 34.1 | vs |
| 24.591 | 24.5 | vs |
| 19.451 | 20.7 | s |
| 22.672 | 18.5 | s |
| 21.918 | 16.1 | s |
| 23.331 | 15.7 | s |
| 14.611 | 13.2 | s |

vs = very strong,
s = strong

DSC analysis of 1-[[5-(aminomethyl)-1-isopentyl-benzimidazol-2-yl]methyl]-3-cyclopropyl-4H-quinazolin-2-one monohydrate showed an initial event with an onset at 70.6° C. and a peak at 96.0° C. followed by a subsequent melt with an onset of 121.2° C. and a peak at 123.9° C. followed by a further event with an onset of 140.0° C. and a peak at 141.3° C. (FIG. 1B). Thus, DSC analysis shows 1-[[5-(aminomethyl)-1-isopentyl-benzimidazol-2-yl]methyl]-3-cyclopropyl-4H-quinazolin-2-one monohydrate is a high melting solid with an onset of melting at about 121.2° C. and a peak at about 123.9° C.

### EXAMPLE 7(II)

### Preparation of 1-[[5-(Aminomethyl)-1-isopentyl-benzimidazol-2-yl]methyl]-3-cyclopropyl-4H-quinazolin-2-one dihydrate

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset).

1-[[5-(aminomethyl)-1-isopentyl-benzimidazol-2-yl]methyl]-3-cyclopropyl-4H-quinazolin-2-one dihydrate was produced by slurrying the 1-[[5-(aminomethyl)-1-isopentyl-benzimidazol-2-yl]methyl]-3-cyclopropyl-4H-quinazolin-2-one monohydrate material in aqueous methanol. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of aqueous methanol added (aqueous methanol consists of approximately 20% to 30% methanol in water). The vial was then sealed tightly with a cap and left to stir on a magnetic stirrer plate. After 3 days, the sample was removed from the plate, the sample was analysed wet by XRPD.

1-[[5-(aminomethyl)-1-isopentyl-benzimidazol-2-yl]methyl]-3-cyclopropyl-4H-quinazolin-2-one dihydrate is characterised by providing an X-ray powder diffraction pattern substantially as shown in FIG. 2. The ten most prominent peaks are shown in Table 2:

TABLE 2
Ten most Prominent X-Ray Powder Diffraction peaks for
1-[[5-(aminomethyl)-1-isopentyl-benzimidazol-2-yl]methyl]-3-
cyclopropyl-4H-quinazolin-2-one dihydrate

| Angle 2- | | Relative |
|---|---|---|
| Theta (2θ) | Intensity % | Intensity |
| 8.850 | 100.0 | vs |
| 8.474 | 39.5 | vs |
| 21.362 | 34.9 | vs |
| 8.173 | 33.8 | vs |
| 17.741 | 30.0 | vs |
| 21.681 | 27.0 | vs |
| 12.578 | 26.6 | vs |
| 22.479 | 20.8 | s |
| 17.225 | 20.7 | s |
| 12.299 | 20.2 | s |

vs = very strong,
s = strong

### EXAMPLE 7(III)

### Preparation of 1-[[5-(Aminomethyl)-1-isopentyl-benzimidazol-2-yl]methyl]-3-cyclopropyl-4H-quinazolin-2-one Form B

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset).

1-8 [5-(aminomethyl)-1-isopentyl-benzimidazol-2-yl]methyl]-3-cyclopropyl-4H-quinazolin-2-one Form B material was produced by slurrying the 1-[[5-(aminomethyl)-1-isopentyl-benzimidazol-2-yl]methyl]-3-cyclopropyl-4H-quinazolin-2-one monohydrate material in ethyl acetate. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of ethyl acetate added. The vial was then sealed tightly with a cap and left to stir on a magnetic stirrer plate. After 7 days, the sample was removed from the plate, the cap taken off and the slurry left to dry under ambient conditions before it was analysed by XRPD and DSC. This form was determined to be crystalline by XRPD and had an initial melting point of 116° C. (onset) followed by an exothermic event, followed by a further melting point of 134° C. TGA analysis indicated a weight loss of approximately 0.46%, suggesting partial solvation.

1-[[5-(aminomethyl)-1-isopentyl-benzimidazol-2-yl]methyl]-3-cyclopropyl-4H-quinazolin-2-one Form B is characterised by providing an X-ray powder diffraction pattern, substantially as shown in FIG. 3A. The ten most prominent peaks are shown in Table 3:

TABLE 3
Ten most Prominent X-Ray Powder Diffraction peaks for
1-[[5-(aminomethyl)-1-isopentyl-benzimidazol-2-
yl]methyl]-3-cyclopropyl-4H-quinazolin-2-one Form B

| Angle 2- | | Relative |
|---|---|---|
| Theta (2θ) | Intensity % | Intensity |
| 19.934 | 100.0 | vs |
| 21.208 | 85.6 | vs |
| 20.335 | 84.8 | vs |
| 8.709 | 81.3 | vs |
| 10.165 | 76.6 | vs |
| 15.465 | 57.8 | vs |
| 11.825 | 52.6 | vs |
| 21.977 | 49.8 | vs |
| 13.023 | 47.6 | vs |
| 13.833 | 43.8 | vs |

vs = very strong,
s = strong

DSC analysis of 1-[[5-(aminomethyl)-1-isopentyl-benzimidazol-2-yl]methyl]-3-cyclopropyl-4H-quinazolin-2-one Form B shows an initial event with an onset at 30.8° C. and a peak at 57.7° C. followed by a subsequent melting endotherm with an onset of 116.1° C. and a peak at 121.0° C., followed by an exothermic event, followed by a further melting endotherm with an onset of 133.7° C. and a peak at 137.6° C. (FIG. 3B).

### EXAMPLE 7(IV)

### Preparation of 1-[[5-(Aminomethyl)-1-isopentyl-benzimidazol-2-yl]methyl]-3-cyclopropyl-4H-quinazolin-2-one Form C

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be is crystalline. This material had a melting point of 121.2° C. (onset).

1-[[5-(aminomethyl)-1-isopentyl-benzimidazol-2-yl]methyl]-3-cyclopropyl-4H-quinazolin-2-one Form C material was produced by slurrying the 1-[[5-(aminomethyl)-1-isopentyl-benzimidazol-2-yl]methyl]-3-cyclopropyl-4H-quinazolin-2-one monohydrate material in acetonitrile. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of acetonitrile added. The vial was then sealed tightly with a cap and left to stir on a magnetic stirrer plate. After 7 days, the sample was removed from the plate, the cap taken off and the slurry left to dry s under ambient conditions before it was analysed by XRPD and DSC. This form was determined to be crystalline by XRPD and had a melting point of 136.9° C. (onset). TGA analysis indicated a weight loss of approximately 4.0%, suggesting partial solvation.

1-[[5-(aminomethyl)-1-isopentyl-benzimidazol-2-yl]methyl]-3-cyclopropyl-4H-quinazolin-2-one Form C is characterised by providing an X-ray powder diffraction pattern, substantially as shown in FIG. 4A. The ten most prominent peaks are shown in Table 4:

TABLE 4
Ten most Prominent X-Ray Powder Diffraction peaks for
1-[[5-(aminomethyl)-1-isopentyl-benzimidazol-2-yl]methyl]-
3-cyclopropyl-4H-quinazolin-2-one monohydrate Form C

| Angle 2- | | Relative |
|---|---|---|
| Theta (2θ) | Intensity % | Intensity |
| 9.839 | 100.0 | vs |
| 6.942 | 64.6 | vs |
| 20.467 | 59.0 | vs |
| 14.297 | 53.3 | vs |
| 22.866 | 48.2 | vs |
| 24.404 | 40.3 | vs |
| 20.889 | 39.7 | vs |
| 24.652 | 35.9 | vs |
| 9.441 | 32.9 | vs |
| 11.192 | 25.5 | vs |

vs = very strong

DSC analysis of 1-[[5-(aminomethyl)-1-isopentyl-benzimidazol-2-yl]methyl]-3-cyclopropyl-4H-quinazolin-2-one Form C shows an initial event with an onset at 56.8° C. and a peak at 85.2° C. followed by a subsequent melting endotherm with an onset of 136.9° C. and a peak at 141.0° C. (FIG. 4B). DSC analysis shows 1-[[5-(aminomethyl)-1-isopentyl-benzimidazol-2-yl]methyl]-3-cyclopropyl-4H-quinazolin-2-one Form C is a high melting solid with an onset of melting at about 136.9° C. and a peak at about 141.0° C.

### EXAMPLE 7(V)

### Preparation of 1-[[5-(Aminomethyl)-1-isopentyl-benzimidazol-2-yl]methyl]-3-cyclopropyl-4H-quinazolin-2-one Form D

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset)

1-[[5-(aminomethyl)-1-isopentyl-benzimidazol-2-yl]methyl]-3-cyclopropyl-4H-quinazolin-2-one monohydrate Form D was produced by slurrying the 1-[[5-(aminomethyl)-1-isopentyl-benzimidazol-2-yl]methyl]-3-cyclopropyl-4H-quinazolin-2-monohydrate material in acetonitrile at 50° C. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of acetonitrile added. The vial was then sealed tightly with a cap and left to stir on a magnetic stirrer plate. After 7 days, the sample was removed from the plate, the cap taken off and the slurry left to dry under ambient conditions before it was analysed by XRPD and DSC. This form was determined to be crystalline by XRPD and had a melting point of 127.3° C. (onset). TGA analysis indicated a weight loss of approximately 2.3%, suggesting partial solvation.

1-[[5-(aminomethyl)-1-isopentyl-benzimidazol-2-yl]methyl]-3-cyclopropyl-4H-quinazolin-2-one Form D is characterised by providing an X-ray powder diffraction pattern, substantially as shown in FIG. 5A. The ten most prominent peaks are shown in Table 5:

TABLE 5
Ten most Prominent X-Ray Powder Diffraction peaks for
1-[[5-(aminomethyl)-1-isopentyl-benzimidazol-2-yl]methyl]-
3-cyclopropyl-4H-quinazolin-2-one Form D

| Angle 2- | | Relative |
|---|---|---|
| Theta (2θ) | Intensity % | Intensity |
| 10.366 | 100.0 | vs |
| 12.195 | 40.6 | vs |
| 18.241 | 30.4 | vs |
| 17.132 | 28.3 | vs |
| 19.611 | 24.6 | vs |
| 7.513 | 22.3 | s |
| 19.306 | 22.0 | s |
| 22.478 | 20.3 | s |
| 20.151 | 19.4 | s |
| 8.734 | 18.8 | s |

vs = very strong,
s = strong

DSC analysis of 1-[[5-(aminomethyl)-1-isopentyl-benzimidazol-2-yl]methyl]-3-cyclopropyl-4H-quinazolin-2-one Form D shows a melting endotherm with an onset at 127.3° C. and a peak at 132.9° C. (FIG. 5B). DSC analysis shows 1-[[5-(aminomethyl)-1-isopentyl-benzimidazol-2-yl]methyl]-3-cyclopropyl-4H-quinazolin-2-one Form D is a high melting solid with an onset of melting at about 127.3° C. and a peak at 132.9° C.

### EXAMPLE 7(VI)

### Preparation of 1-[[5-(Aminomethyl)-1-isopentyl-benzimidazol-2-yl]methyl]-3-cyclopropyl-4H-quinazolin-2-one Form E

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset).

1-[[5-(aminomethyl)-1-isopentyl-benzimidazol-2-yl]methyl]-3-cyclopropyl-4H-quinazolin-2-one monohydrate Form E material was produced by slurrying the 1-[[5-(aminomethyl)-1-isopentyl-benzimidazol-2-yl]methyl]-3-cyclopropyl-4H-quinazolin-2-one monohydrate material in methanol. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of methanol added. The vial was then sealed tightly with a cap and left to stir on a magnetic stirrer plate. After 7 days, the sample was removed from the plate, the cap taken off and the slurry left to dry under ambient conditions before it was analysed by XRPD and DSC. This form was determined to be crystalline by XRPD and had a melting point of 69.5° C. (onset). TGA analysis indicated a weight loss of approximately 3.4%, suggesting partial solvation.

1-[[5-(aminomethyl)-1-isopentyl-benzimidazol-2-yl]methyl]-3-cyclopropyl-4H-quinazolin-2-one Form E is characterised by providing an X-ray powder diffraction pattern, substantially as shown in FIG. 6A. The ten most prominent peaks are shown in Table 6:

TABLE 6
Ten most Prominent X-Ray Powder Diffraction peaks for
1-[[5-(aminomethyl)-1-isopentyl-benzimidazol-2-yl]methyl]-
3-cyclopropyl-4H-quinazolin-2-one Form E

| Angle 2- | | Relative |
|---|---|---|
| Theta (2θ) | Intensity % | Intensity |
| 8.683 | 100.0 | vs |
| 21.570 | 79.3 | vs |
| 17.243 | 36.4 | vs |
| 8.243 | 32.0 | vs |
| 12.826 | 25.7 | vs |
| 18.735 | 20.3 | s |
| 12.262 | 16.7 | s |
| 19.700 | 13.5 | s |
| 24.777 | 12.4 | s |
| 19.286 | 12.4 | s |

vs = very strong,
s = strong

DSC analysis of 1-[[5-(aminomethyl)-1-isopentyl-benzimidazol-2-yl]methyl]-3-cyclopropyl-4H-quinazolin-2-one Form E shows a melting endotherm with an onset at 69.5° C. and a peak at 94.0° C. (FIG. 6B). DSC analysis shows 1-[[5-(aminomethyl)-1-isopentyl-benzimidazol-2-yl]methyl]-3-cyclopropyl-4H-quinazolin-2-one Form E is a high melting solid with an onset of melting at about 69.5° C. and a peak at about 94.0° C.

### EXAMPLE 7(VII)

### Preparation of 1-[[5-(Aminomethyl)-1-isopentyl-benzimidazol-2-yl]methyl]-3-cyclopropyl-4H-quinazolin-2-one Form F

The X-ray powder diffraction spectra for 1-[[5-(aminomethyl)-1-isopentyl-benzimidazol-2-yl]methyl]-3-cyclopropyl-4H-quinazolin-2-one Form D showed the material to be crystalline. This material had a melting point of 127.3° C. (onset).

1-[[5-(Aminomethyl)-1-isopentyl-benzimidazol-2-yl]methyl]-3-cyclopropyl-4H-quinazolin-2-one Form F material was produced by slurrying the 1-[[5-(aminomethyl)-1-isopentyl-benzimidazol-2-yl]methyl]-3-cyclopropyl-4H-quinazolin-2-one Form D material in aqueous methanol (aqueous methanol consists of approximately 20% to 30% methanol in water). Approximately 20 mg of the 1-[[5-(aminomethyl)-1-isopentyl-benzimidazol-2-yl]methyl]-3-cyclopropyl-4H-quinazolin-2-one Form D material was placed in a vial with a magnetic flea, and approximately 2 ml of aqueous methanol added. The vial was then sealed tightly with a cap and left to stir on a magnetic stirrer plate. After 7 days, the sample was removed from the plate, the cap taken off and the slurry left to dry under ambient conditions before it was analysed by XRPD and DSC. This form was determined to be crystalline by XRPD and had a melting point of 132.0° C. (onset).

1-[[5-(Aminomethyl)-1-isopentyl-benzimidazol-2-yl]methyl]-3-cyclopropyl-4H-quinazolin-2-one Form F is characterised by providing an X-ray powder diffraction pattern, substantially as shown in FIG. 7A. The seven most prominent peaks are shown in Table 7:

TABLE 7
Seven most Prominent X-Ray Powder Diffraction peaks for
1-[[5-(Aminomethyl)-1-isopentyl-benzimidazol-2-yl]methyl]-
3-cyclopropyl-4H-quinazolin-2-one Form F

| Angle 2- | | Relative |
|---|---|---|
| Theta (2θ) | Intensity % | Intensity |
| 8.831 | 100.0 | vs |
| 8.396 | 91.7 | vs |
| 8.164 | 88.6 | vs |
| 4.119 | 69.5 | vs |
| 21.350 | 55.1 | vs |
| 22.502 | 42.9 | vs |
| 21.762 | 40.2 | vs |

vs = very strong

DSC analysis of 1-[[5-(aminomethyl)-1-isopentyl-benzimidazol-2-yl]methyl]-3-cyclopropyl-4H-quinazolin-2-one Form F shows an initial event with an onset at 58.1° C. and a peak at 80.9° C. followed by a subsequent melt with an onset of 132.0° C. and a peak at 136.1° C. (FIG. 7B). DSC analysis shows 1-[[5-(aminomethyl)-1-isopentyl-benzimidazol-2-yl]methyl]-3-cyclopropyl-4H-quinazolin-2-one Form F is a high melting solid with an onset of melting at about 132.0° C. and a peak at about 136.1° C.

### EXAMPLE 7(VIII)

### Preparation of 1-[[5-(Aminomethyl)-1-isopentyl-benzimidazol-2-yl]methyl]-3-cyclopropyl-4H-quinazolin-2-one Form G

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset).

1-[[5-(Aminomethyl)-1-isopentyl-benzimidazol-2-yl]methyl]-3-cyclopropyl-4H-quinazolin-2-one Form G material was produced by placing approximately 20 mg of the 1-[[5-(aminomethyl)-1-isopentyl-benzimidazol-2-yl]methyl]-3-cyclopropyl-4H-quinazolin-2-one monohydrate material in a vial and storing in a vacuum oven for approximately 2 days at 60° C. The resulting material was analysed by XRPD and DSC. This form was determined to be crystalline by XRPD and had a melting point of 112.9° C. (onset).

1-[[5-(Aminomethyl)-1-isopentyl-benzimidazol-2-yl]methyl]-3-cyclopropyl-4H-quinazolin-2-one Form G is characterised by providing an X-ray powder diffraction pattern, substantially as shown in FIG. 8A. The ten most prominent peaks are shown in Table 8:

TABLE 8
Ten most Prominent X-Ray Powder Diffraction peaks for
1-[[5-(aminomethyl)-1-isopentyl-benzimidazol-2-yl]methyl]-
3-cyclopropyl-4H-quinazolin-2-one Form G

| Angle 2- | | Relative |
|---|---|---|
| Theta (2θ) | Intensity % | Intensity |
| 10.364 | 100.0 | vs |
| 12.180 | 65.5 | vs |
| 19.612 | 59.0 | vs |
| 18.199 | 58.3 | vs |
| 17.171 | 52.1 | vs |
| 21.893 | 51.0 | vs |
| 24.551 | 45.3 | vs |
| 9.748 | 40.5 | vs |
| 7.502 | 34.4 | vs |
| 25.709 | 32.6 | vs |

vs = very strong

DSC analysis of 1-[[5-(aminomethyl)-1-isopentyl-benzimidazol-2-yl]methyl]-3-cyclopropyl-4H-quinazolin-2-one Form G shows an initial event with an onset at 76.8° C. and a peak at 86.1° C. followed by a subsequent melt with an onset of 112.9° C. and a peak at 130.8° C. (FIG. 8B). DSC analysis shows 1-[[5-(aminomethyl)-1-isopentyl-benzimidazol-2-yl]methyl]-3-cyclopropyl-4H-quinazolin-2-one Form G is a high melting solid with an onset of melting at about 112.9° C. and a peak at about 130.8° C.

### EXAMPLE 7(IX)

### Preparation of 1-[[5-(Aminomethyl)-1-isopentyl-benzimidazol-2-yl]methyl]-3-cyclopropyl-4H-quinazolin-2-one Form H

The X-ray powder diffraction spectra for 1-[[5-(aminomethyl)-1-isopentyl-benzimidazol-2-yl]methyl]-3-cyclopropyl-4H-quinazolin-2-one monohydrate showed the material to be crystalline. This material had a melting point of 121.2° C. (onset).

1-[[5-(aminomethyl)-1-isopentyl-benzimidazol-2-yl]methyl]-3-cyclopropyl-4H-quinazolin-2-one Form H material was produced by slurrying the 1-[[5-(aminomethyl)-1-isopentyl-benzimidazol-2-yl]methyl]-3-cyclopropyl-4H-quinazolin-2-one monohydrate material in isopropyl alcohol. Approximately 20 mg of the 1-[[5-(aminomethyl)-1-isopentyl-benzimidazol-2-yl]methyl]-3-cyclopropyl-4H-quinazolin-2-one monohydrate material was placed in a vial with a magnetic flea, and approximately 2 ml of methanol added. The vial was then sealed tightly with a cap and left to stir on a magnetic stirrer is plate. After 3 days, the sample was removed from the plate, the cap taken off and the slurry left to dry under ambient conditions before it was analysed by XRPD and DSC. This form was determined to be crystalline by XRPD and had a melting point of 75.0° C. (onset).

1-[[5-(aminomethyl)-1-isopentyl-benzimidazol-2-yl]methyl]-3-cyclopropyl-4H-quinazolin-2-one Form H is characterised by providing an X-ray powder diffraction pattern, substantially as shown in FIG. 9A. The ten most prominent peaks are shown in Table 9:

TABLE 9
Ten most Prominent X-Ray Powder Diffraction peaks for
1-[[5-(aminomethyl)-1-isopentyl-benzimidazol-2-yl]methyl]-
3-cyclopropyl-4H-quinazolin-2-one Form H

| Angle 2- | | Relative |
|---|---|---|
| Theta (2θ) | Intensity % | Intensity |
| 19.869 | 100.0 | vs |
| 9.758 | 85.0 | vs |
| 21.926 | 62.7 | vs |
| 13.543 | 59.4 | vs |
| 25.630 | 26.4 | vs |
| 13.850 | 23.6 | s |
| 17.233 | 17.6 | s |
| 10.237 | 16.4 | s |
| 22.873 | 14.6 | s |
| 13.111 | 12.1 | s |

vs = very strong,
s = strong

DSC analysis of 1-[[5-(aminomethyl)-1-isopentyl-benzimidazol-2-yl]methyl]-3-cyclopropyl-4H-quinazolin-2-one Form H shows a melting endotherm with an onset at 75.0° C. and a peak at 78.9° C. (FIG. 9B). Thus DSC analysis shows 1-[[5-(aminomethyl)-1-isopentyl-benzimidazol-2-yl]methyl]-3-cyclopropyl-4H-quinazolin-2-one Form H is a high melting solid with an onset of melting at about 75.0° C. and a peak at about 78.9° C.

### EXAMPLE7(X)

### Preparation of 1-[[5-(Aminomethyl)-1-isopentyl-benzimidazol-2-yl]methyl]-3-cyclopropyl-4H-quinazolin-2-one Acetate Salt Form A

100 mg of 1-[[5-(aminomethyl)-1-isopentyl-benzimidazol-2-yl]methyl]-3-cyclopropyl-4H-quinazolin-2-one was dissolved in 2 ml of acetonitrile with gentle heating to aid dissolution. This was added to a solution of acetic acid (1.1 molar equivalent); this is resultant mixture was heated gently and stirred for 10 minutes then allowed to cool. On cooling a solid precipitated out, this was then left to stir at ambient temperature overnight, after which the precipitate was filtered and analysed by XRPD to give a material with a powder pattern as shown in FIG. 10 with peaks listed in Table 10. The XRPD diffractogram differed from any of the known forms of the free form.

NMR data indicated a 1.0:1 molar equivalent of acetate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one acetate salt Form A is characterised in providing an X-ray powder diffraction pattern, substantially as shown in FIG. 10. The most prominent peaks are shown in Table 10:

TABLE 10
Most Prominent X-Ray Powder Diffraction peaks for
1-[[5-(Aminomethyl)-1-isopentyl-benzimidazol-2-yl]methyl]-
3-cyclopropyl-4H-quinazolin-2-one acetate salt Form A

| Angle/° 2θ (λ = 1.5418 Å) | d-spacing/Å | Relative Intensity/% |
|---|---|---|
| 22.4 | 4.0 | 100 |
| 12.6 | 7.0 | 74 |
| 27.7 | 3.2 | 59 |
| 18.1 | 4.9 | 55 |
| 20.7 | 4.3 | 54 |
| 6.9 | 12.7 | 49 |
| 18.8 | 4.7 | 49 |
| 9.4 | 9.4 | 46 |
| 10.9 | 8.1 | 41 |
| 13.8 | 6.4 | 39 |

### EXAMPLE 7(XI)

### Preparation of 1-[[5-(Aminomethyl)-1-isopentyl-benzimidazol-2-yl]methyl]-3-cyclopropyl-4H-quinazolin-2-one Acetate Salt Form B

10-30 mg of 1-[[5-(aminomethyl)-1-isopentyl-benzimidazol-2-yl]methyl]-3-cyclopropyl-4H-quinazolin-2-one acetate salt Form A was added to a sample vial, along with a sufficient volume of methanol to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 11 with peaks listed in Table 11. The XRPD diffractogram differed from any of the known forms of the free form.

NMR data indicated a 2.1:1 molar equivalent of acetate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one acetate salt Form B is characterised in providing an X-ray powder diffraction pattern, substantially as shown in FIG. 11. The most prominent peaks are shown in Table 11:

TABLE 11
Most Prominent X-Ray Powder Diffraction peaks for
1-[[5-(Aminomethyl)-1-isopentyl-benzimidazol-2-
yl]methyl]-3-cyclopropyl-4H-quinazolin-2-one acetate salt Form B

| Angle/° 2θ (λ = 1.5418 Å) | d-spacing/Å | Relative Intensity/% |
|---|---|---|
| 8.2 | 10.7 | 100 |
| 9.4 | 9.4 | 37 |
| 14.7 | 6.0 | 35 |
| 19.8 | 4.5 | 35 |
| 4.1 | 21.4 | 32 |
| 7.3 | 12.0 | 31 |
| 14.1 | 6.3 | 29 |
| 18.8 | 4.7 | 28 |
| 22.1 | 4.0 | 28 |
| 12.5 | 7.1 | 25 |

### EXAMPLE 7(XII)

### Preparation of 1-[[5-(Aminomethyl)-1-isopentyl-benzimidazol-2-yl]methyl]-3-cyclopropyl-4H-quinazolin-2-one Adipate Salt Form A

100 mg of 1-[[5-(aminomethyl)-1-isopentyl-benzimidazol-2-yl]methyl]-3-cyclopropyl-4H-quinazolin-2-one was dissolved in 2 ml of acetonitrile with gentle heating to aid dissolution. This was added to a solution of adipic acid (1.1 molar equivalent) in 3 mls of acetonitrile. The resultant mixture was gently heated and stirred for 10 minutes then allowed to cool. On cooling a solid precipitated out, this was then left to stir at ambient temperature overnight, after which the precipitate was filtered and analysed by XRPD to give a material with a powder pattern as shown in FIG. 12 with peaks listed in Table 12. The XRPD diffractogram differed from any of the known forms of the free form. HPLC data indicated that the assay of the material was 99% with respect to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one adipate salt Form A is characterised in providing an X-ray powder diffraction pattern, substantially as shown in FIG. 12. The most prominent peaks are shown in Table 12:

TABLE 12
Most Prominent X-Ray Powder Diffraction peaks for
1-[[5-(Aminomethyl)-1-isopentyl-benzimidazol-2-
yl]methyl]-3-cyclopropyl-4H-quinazolin-2-one adipate salt Form A

| Angle/° 2θ (λ = 1.5418 Å) | d-spacing/Å | Relative Intensity/% |
|---|---|---|
| 18.2 | 4.9 | 100 |
| 22.6 | 3.9 | 73 |
| 12.7 | 7.0 | 63 |
| 21.6 | 4.1 | 60 |
| 9.4 | 9.4 | 57 |
| 22.0 | 4.0 | 52 |
| 18.7 | 4.7 | 50 |
| 20.6 | 4.3 | 44 |
| 9.7 | 9.1 | 37 |
| 11.8 | 7.5 | 36 |

### EXAMPLE 7(XIII)

### Preparation of 1-[[5-(Aminomethyl)-1-isopentyl-benzimidazol-2-yl]methyl]-3-cyclopropyl-4H-quinazolin-2-one Adipate Salt Form B

The preparation of Example 7(xii) above was repeated except after the precipitate was filtered and analysed by XRPD a material with a powder pattern as shown in FIG. 13 with peaks listed in Table 13 was provided. The XRPD diffractogram differed from any of the known forms of the free form.

NMR data indicated a 1.2:1 molar equivalent of adipate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one adipate salt Form B is characterised in providing an X-ray powder diffraction pattern, substantially as shown in FIG. 13. The most prominent peaks are is shown in Table 13:

TABLE 13
Most Prominent X-Ray Powder Diffraction peaks for
1-[[5-(Aminomethyl)-1-isopentyl-benzimidazol-2-
yl]methyl]-3-cyclopropyl-4H-quinazolin-2-one adipate salt Form B

| Angle/° 2θ (λ = 1.5418 Å) | d-spacing/Å | Relative Intensity/% |
|---|---|---|
| 9.1 | 9.7 | 100 |
| 22.0 | 4.0 | 63 |
| 11.8 | 7.5 | 50 |
| 22.4 | 4.0 | 39 |
| 10.7 | 8.2 | 39 |
| 23.5 | 3.8 | 30 |
| 24.1 | 3.7 | 27 |
| 6.0 | 14.6 | 25 |

### EXAMPLE 7(XIV)

### Preparation of 1-[[5-(Aminomethyl)-1-isopentyl-benzimidazol-2-yl]methyl]-3-cyclopropyl-4H-quinazolin-2-one Adipate Salt Form C

10-30 mg of 1-[[5-(Aminomethyl)-1-isopentyl-benzimidazol-2-yl]methyl]-3-cyclopropyl-4H-quinazolin-2-one adipate salt Form B material was added to a sample vial, along with a sufficient volume of water to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 14 with peaks listed in Table 14. The XRPD diffractogram differed from any of the known forms of the free form.

1-[[5-(Aminomethyl)-1-isopentyl-benzimidazol-2-yl]methyl]-3-cyclopropyl-4H-quinazolin-2-one adipate salt Form C is characterised in providing an X-ray powder diffraction pattern, substantially as shown in FIG. 14. The most prominent peaks are shown in Table 14:

TABLE 14
Most Prominent X-Ray Powder Diffraction peaks for
1-[[5-(Aminomethyl)-1-isopentyl-benzimidazol-2-
yl]methyl]-3-cyclopropyl-4H-quinazolin-2-one adipate salt Form C

| Angle/° 2θ (λ = 1.5418 Å) | d-spacing/Å | Relative Intensity/% |
|---|---|---|
| 23.3 | 3.8 | 100 |
| 9.4 | 9.4 | 84 |
| 10.3 | 8.6 | 84 |
| 22.6 | 3.9 | 77 |
| 18.3 | 4.8 | 69 |
| 21.6 | 4.1 | 69 |
| 23.0 | 3.9 | 69 |
| 25.4 | 3.5 | 61 |
| 18.7 | 4.7 | 61 |
| 12.7 | 7.0 | 59 |

### EXAMPLE 7(XV)

### Preparation of 1-[[5-(Aminomethyl)-1-isopentyl-benzimidazol-2-yl]methyl]-3-cyclopropyl-4H-quinazolin-2-one Cinnamate Salt Form A

100 mg of 1-[[5-(aminomethyl)-1-isopentyl-benzimidazol-2-yl]methyl]-3-cyclopropyl-4H-quinazolin-2-one was dissolved in 2 ml of acetonitrile with gentle heating to aid dissolution. This was added to a solution of cinnamic acid (1.1 molar equivalent) in 2 ml of acetonitrile. The resultant mixture was heated gently and stirred for 10 minutes then allowed to cool. On cooling a solid precipitated out, this was then left to stir at ambient temperature overnight, after which the precipitate was filtered and analysed by XRPD to give a material with a powder pattern as shown in FIG. 15 with peaks listed in Table 15. The XRPD diffractogram differed from any of the known forms of the free form.

NMR data indicated a 1.0:1 molar equivalent of cinnamate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one cinnamate salt Form A is characterised in providing an X-ray powder diffraction pattern, substantially as shown in FIG. 15. The most prominent peaks are shown in Table 15:

TABLE 15
Most Prominent X-Ray Powder Diffraction peaks for
1-[[5-(Aminomethyl)-1-isopentyl-benzimidazol-2-
yl]methyl]-3-cyclopropyl-4H-quinazolin-2-one cinnamate salt Form A

| Angle/° 2θ (λ = 1.5418 Å) | d-spacing/Å | Relative Intensity/% |
|---|---|---|
| 9.3 | 9.5 | 100 |
| 17.7 | 5.0 | 29 |
| 5.3 | 16.8 | 27 |
| 16.9 | 5.2 | 22 |
| 13.5 | 6.5 | 20 |
| 17.2 | 5.1 | 17 |
| 22.0 | 4.0 | 15 |
| 14.5 | 6.1 | 15 |
| 8.8 | 10.0 | 15 |
| 24.2 | 3.7 | 15 |

### EXAMPLE 7(XVI)

### Preparation of 1-[[5-(Aminomethyl)-1-isopentyl-benzimidazol-2-yl]methyl]-3-cyclopropyl-4H-quinazolin-2-one Cinnamate Salt Form B

10-30 mg of 1-[[5-(Aminomethyl)-1-isopentyl-benzimidazol-2-yl]methyl]-3-cyclopropyl-4H-quinazolin-2-one cinnamate salt Form A material was added to a sample vial, along with a sufficient volume of water to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 16 with peaks listed in Table 16. The XRPD diffractogram differed from any of the known forms of the free form.

1-[[5-(Aminomethyl)-1-isopentyl-benzimidazol-2-yl]methyl]-3-cyclopropyl-4H-quinazolin-2-one cinnamate salt Form B is characterised in providing an X-ray powder diffraction pattern, substantially as shown in FIG. 16. The most prominent peaks are shown in Table 16:

TABLE 16
Most Prominent X-Ray Powder Diffraction peaks for
1-[[5-(Aminomethyl)-1-isopentyl-benzimidazol-2-
yl]methyl]-3-cyclopropyl-4H-quinazolin-2-one cinnamate salt Form B

| Angle/° 2θ (λ = 1.5418 Å) | d-spacing/Å | Relative Intensity/% |
|---|---|---|
| 23.5 | 3.8 | 100 |
| 7.0 | 12.7 | 94 |
| 20.9 | 4.2 | 77 |
| 12.7 | 6.9 | 74 |
| 19.3 | 4.6 | 66 |
| 26.4 | 3.4 | 51 |
| 17.4 | 5.1 | 50 |
| 9.9 | 9.0 | 49 |
| 24.1 | 3.7 | 44 |
| 14.8 | 6.0 | 44 |

### EXAMPLE 7(XVII)

### Preparation of 1-[[5-(Aminomethyl)-1-isopentyl-benzimidazol-2-yl]methyl]-3-cyclopropyl-4H-quinazolin-2-one Stearate Salt Form A

100 mg of 1-[[5-(aminomethyl)-1-isopentyl-benzimidazol-2-yl]methyl]-3-cyclopropyl-4H-quinazolin-2-one was dissolved in 2 ml of acetonitrile with heating to aid dissolution. This was added to a solution of stearic acid (1.1 molar equivalent) in 2 mls of acetonitrile. The resultant mixture was heated and stirred for 10 minutes then allowed to cool. On cooling a solid precipitated out, this was then left to stir at ambient temperature overnight, after which the precipitate was filtered and analysed by XRPD to give a material with a powder pattern as shown in FIG. 17 with peaks listed in Table 17. The XRPD diffractogram differed from any of the known forms of the free form.

NMR data indicated a 1.2:1 molar equivalent of stearate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one stearate salt Form A is characterised in providing an X-ray powder diffraction pattern, substantially as shown in FIG. 17. The most prominent peaks are shown in Table 17:

TABLE 17
Most Prominent X-Ray Powder Diffraction peaks for
1-[[5-(Aminomethyl)-1-isopentyl-benzimidazol-2-
yl]methyl]-3-cyclopropyl-4H-quinazolin-2-one stearate salt Form A

| Angle/° 2θ (λ = 1.5418 Å) | d-spacing/Å | Relative Intensity/% |
|---|---|---|
| 3.0 | 29.2 | 100 |
| 4.5 | 19.6 | 52 |
| 6.3 | 14.0 | 40 |
| 5.4 | 16.3 | 23 |
| 7.5 | 11.8 | 22 |
| 7.8 | 11.3 | 21 |
| 9.7 | 9.1 | 20 |
| 19.4 | 4.6 | 20 |
| 14.7 | 6.0 | 19 |
| 10.4 | 8.5 | 18 |

### EXAMPLE 7(XVIII)

### Preparation of 1-[[5-(Aminomethyl)-1-isopentyl-benzimidazol-2-yl]methyl]-3-cyclopropyl-4H-quinazolin-2-one Stearate Salt Form B

10-30 mg of 1-[[5-(aminomethyl)-1-isopentyl-benzimidazol-2-yl]methyl]-3-cyclopropyl-4H-quinazolin-2-one stearate salt Form A material was added to a sample vial, along with a sufficient volume of water to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 18 with peaks listed in Table 18. The XRPD diffractogram differed from any of the known forms of the free form.

1-[[5-(Aminomethyl)-1-isopentyl-benzimidazol-2-yl]methyl]-3-cyclopropyl-4H-quinazolin-2-one stearate salt Form B is characterised in providing an X-ray powder diffraction pattern, substantially as shown in FIG. 18. The most prominent peaks are shown in Table 18:

TABLE 18
Most Prominent X-Ray Powder Diffraction peaks for
1-[[5-(Aminomethyl)-1-isopentyl-benzimidazol-2-
yl]methyl]-3-cyclopropyl-4H-quinazolin-2-one stearate salt Form B

| Angle/° 2θ (λ = 1.5418 Å) | d-spacing/Å | Relative Intensity/% |
|---|---|---|
| 2.6 | 34.1 | 100 |
| 10.2 | 8.6 | 21 |
| 6.3 | 14.1 | 18 |
| 5.1 | 17.1 | 18 |
| 7.7 | 11.4 | 16 |
| 15.4 | 5.7 | 15 |
| 5.4 | 16.2 | 13 |

### EXAMPLE 7(XIX)

### Preparation of 1-[[5-(Aminomethyl)-1-isopentyl-benzimidazol-2-yl]methyl]-3-cyclopropyl-4H-quinazolin-2-one Stearate Salt Form C

10-30 mg of 1-[[5-(Aminomethyl)-1-isopentyl-benzimidazol-2-yl]methyl]-3-cyclopropyl-4H-quinazolin-2-one Stearate Salt Form A material was added to a sample vial, along with a sufficient volume of methanol to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 19 with peaks listed in Table 19. The XRPD diffractogram differed from any of the known forms of the free form.

NMR data indicated a 1.2:1 molar equivalent of stearate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one stearate salt Form C is characterised in providing an X-ray powder diffraction pattern, substantially as shown in FIG. 19. The most prominent peaks are shown in Table 19:

TABLE 19
Most Prominent X-Ray Powder Diffraction peaks for
1-[[5-(Aminomethyl)-1-isopentyl-benzimidazol-2-
yl]methyl]-3-cyclopropyl-4H-quinazolin-2-one stearate salt Form C

| Angle/° 2θ (λ = 1.5418 Å) | d-spacing/Å | Relative Intensity/% |
|---|---|---|
| 3.6 | 24.2 | 100 |
| 5.5 | 16.1 | 88 |
| 23.3 | 3.8 | 82 |
| 8.4 | 10.5 | 77 |
| 10.2 | 8.7 | 73 |

### EXAMPLE 7(XX)

### Preparation of 1-[[5-(Aminomethyl)-1-isopentyl-benzimidazol-2-yl]methyl]-3-cyclopropyl-4H-quinazolin-2-one Stearate Salt Form D

10-30 mg of 1-[[5-(aminomethyl)-1-isopentyl-benzimidazol-2-yl]methyl]-3-cyclopropyl-4H-quinazolin-2-one Stearate Salt Form A material was added to a sample vial, along with a sufficient volume of ethyl acetate to achieve mobility without completely dissolving the sample. A magnetic flea was then added and the vial was placed on the stirrer plate at ambient temperature to stir at approximately 200-300 rpm for 2 weeks. The precipitate was filtered and analysed by XRPD to give a material with a powder pattern as shown in FIG. 20 with peaks listed in Table 20. The XRPD diffractogram differed from any of the known forms of the free form.

NMR data indicated a 2.0:1 molar equivalent of stearate to 1-[[5-(aminomethyl)-1-isopentyl-benzimidazol-2-yl]methyl]-3-cyclopropyl-4H-quinazolin-2-one.

1-[[5-(Aminomethyl)-1-isopentyl-benzimidazol-2-yl]methyl]-3-cyclopropyl-4H-quinazolin-2-one stearate salt Form D is characterised in providing an X-ray powder diffraction pattern, substantially as shown in FIG. 20. The most prominent peaks are shown in Table 20:

TABLE 20
Most Prominent X-Ray Powder Diffraction peaks for
1-[[5-(Aminomethyl)-1-isopentyl-benzimidazol-2-
yl]methyl]-3-cyclopropyl-4H-quinazolin-2-one stearate salt Form D

| Angle/° 2θ (λ = 1.5418 Å) | d-spacing/Å | Relative Intensity/% |
|---|---|---|
| 2.3 | 38.5 | 100 |
| 4.9 | 17.9 | 59 |
| 9.2 | 9.6 | 31 |
| 24.2 | 3.7 | 30 |
| 6.7 | 13.1 | 28 |
| 21.7 | 4.1 | 27 |
| 19.8 | 4.5 | 27 |
| 7.0 | 12.6 | 26 |
| 16.4 | 5.4 | 25 |
| 5.6 | 15.7 | 24 |

### EXAMPLES 24 TO 27

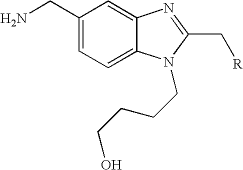

| # | R | ¹H NMR | LC/MS |
|---|---|---|---|
| 24 | 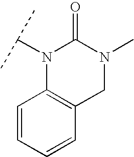 | (250 MHz, DMSO-d₆): δ 1.42(m, 2H), 1.70(m, 2H), 2.94(s, 3H), 3.41(t, 2H), 3.92(s, 2H), 4.29 (t, 2H), 4.45(s, 2H), 5.32(s, 2H), 6.91(t, 1H), 6.95(d, 1H) 7.15(dd, 1H), 7.19(dd, 1H), 7.52 (m, 2H), 8.38(s, 1H). | 394 (MH⁺) |
| 25 | 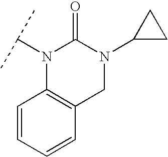 | (250 MHz, DMSO-d₆): δ 0.58(m, 2H), 0.75(m, 2H), 1.49(m, 2H), 1.75(m, 2H), 2.66(m, 1H), 3.42(t, 2H), 3.93(s, 2H), 4.30(t, 2H), 4.40(s, 2H), 5.33(s, 2H), 6.91(m, 1H), 7.12(m, 2H), 7.21(m, 2H), 7.51(m, 2H), 8.38(m, 1H) | 421 (MH⁺) |
| 26 | 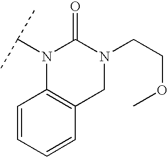 | (250 MHz, DMSO-d6): δ 1.46(m, 2H), 1.74(m, 2H), 3.27(s, 3H), 3.41(t, 2H), 3.55(s, 4H), 3.93 (s, 2H), 4.28(t, 2H), 4.53(s, 2H), 5.33(s, 2H), 6.93(m, 1H), 7.12(m, 2H), 7.22(m, 2H), 7.52 (m, 2H), 8.39(br.s, 1H) | 439 (MH⁺) |
| 27 | 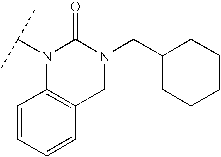 | (250 MHz, DMSO-d₆): δ 0.97(2H, m), 1.15(m, 2H), 1.46(m, 2H), 1.66(m, 8H), 3.25(d, 2H), 3.42(t, 2H), 3.87(d, 2H), 3.97(s, 2H), 4.28(t, 2H), 5.34(s, 2H), 6.91(m, 1H), 7.12(m, 2H), 7.25(m, 2H), 7.51(m, 2H), 8.36(s, 1H) | 477 (MH⁺) |

Examples 24 to 27 were prepared using a procedure analogous to that described in WO 03/053344 (page 21; Scheme XIV)

The process involved coupling of an appropriately bis-protected aminomethyl-alcohol with the indicated quinazolinone, followed by deprotection:

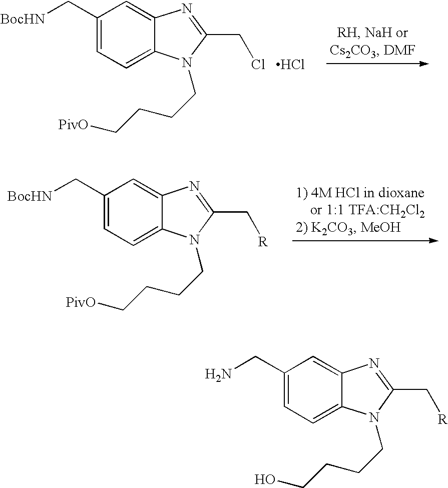

Starting materials for Examples 24 to 27:

### 4-[5-[(tert-Butoxycarbonylamino)methyl]-2-(chloromethyl)benzimidazol-1-yl]butyl 2,2-dimethylpropanoate, HCl salt

Prepared using the procedure described in WO 03/053344 (page 97).

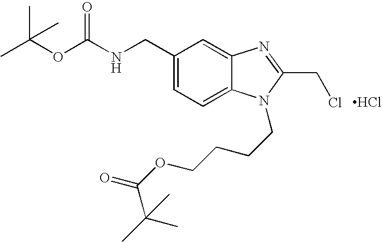

The synthesis of precursors to 4-[5-[(tert-Butoxycarbonylamino)methyl]-2-(chloromethyl)benzimidazol-1-yl]butyl 2,2-dimethylpropanoate, HCl salt, are also described in WO 03/053344.

### EXAMPLES 28 TO 30

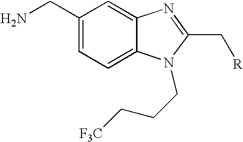

| | | | |
|---|---|---|---|
| # | R | ¹H NMR | LC/MS |

| | | | |
|---|---|---|---|
| 28 | 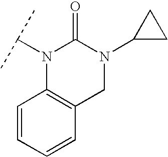 | (250 MHz, DMSO-d₆): δ 0.58(m, 2H), 1.99(m, 2H), 2.41(m, 2H), 2.64(m, 1H), 3.77(s, 2H), 4.39(m, 4H), 5.30(s, 2H), 6.97(m, 1H), 7.21 (m, 4H), 7.51(m, 2H) | 458 (MH⁺) |
| 29 | 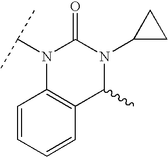 | (250 MHz, DMSO-d₆): δ 0.51(m, 2H), 0.67(m, 2H), 0.82(m, 1H), 1.36(d, 3H), 1.99(m, 2H), 2.43(m, 2H), 2.65(m, 1H), 3.97(s, 2H), 4.34(m, 2H), 4.50 (m, 2H), 4.99(m, 2H), 6.96(m, 1H), 7.11(m, 1H), 7.13(m, 1H), 7.17(m, 1H), 7.26(m, 1H), 7.58(m, 2H) | 471 (MH⁺) |
| 30 | 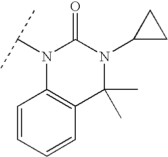 | (250 MHz, DMSO-d₆): δ 0.47(m, 2H), 0.80(m, 2H), 1.63(s, 6H), 2.00(m, 2H), 2.36(m, 2H), 2.42(m, 1H), 3.96(s, 2H), 4.43(t, 2H), 5.35(s, 2H), 7.00(m, 1H), 7.11(m, 1H), 7.16(m, 1H), 7.29(m, 2H), 7.52 (s, 1H), 7.60(d, 1H) | 486 (MH⁺) |

Examples 28 to 30 were prepared in the same manner as Examples 5 to 23 but substituting the appropriate benzimidazole derivative. The process involved coupling of an appropriately protected aminomethyl derivative with the indicated quinazolinone followed by deprotection:

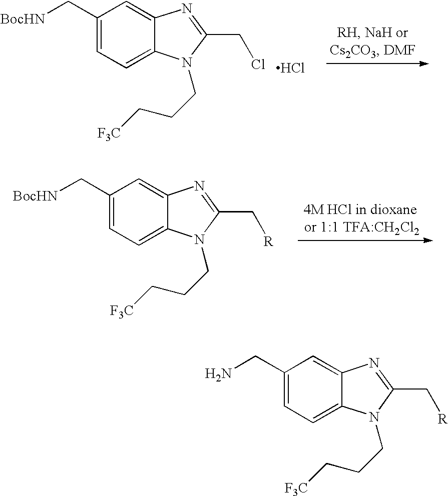

Example 28 was also prepared by reduction of the corresponding cyano derivative:

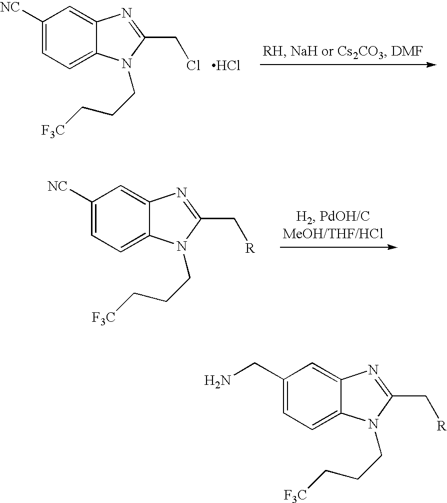

Typical synthesis using a protected aminomethyl derivative:

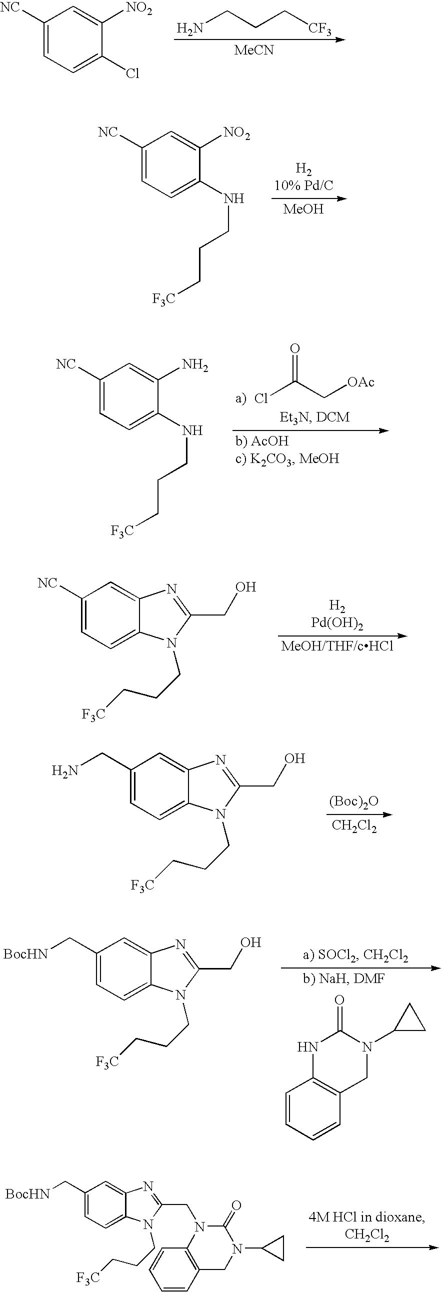

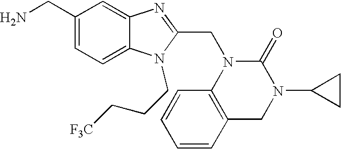

### 3-Nitro-4-(4,4,4-trifluorobutylamino)benzonitrile

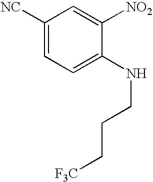

A mixture of 4-chloro-3-nitro-benzonitrile (1.36 g, 7.43 mmol, 1 eq), 4,4,4-trifluorobutan-1-amine (945 mg, 7.43 mmol, 1 eq) and triethylamine (1.04 mL, 7.43 mmol, 1 eq) in acetonitrile (30 mL) was stirred at room temperature. After 16 h the reaction was concentrated in vacuo to dryness. The residue was dissolved in CH₂Cl₂ (30 mL), washed with water (3×15 mL), dried (MgSO₄), filtered and concentrated in vacuo to yield the title compound as a yellow solid (1.99 g, 98%).

¹H NMR (250 MHz, CDCl₃): δ2.10 (m, 2H), 2.33 (m, 2H), 3.51 (q, 2H), 6.95 (d, 1H), 7.68 (m, 1H), 8.44 (br.s, 1H), 8.57 (d, 1H). LC/MS 274 (MH⁻).

### 3-Amino-4-(4,4,4-trifluorobutylamino)benzonitrile

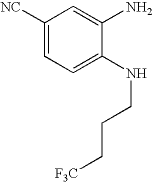

A mixture of 3-nitro-4-(4,4,4-trifluorobutylamino)benzonitrile (1.99 g, 7.29 mmol, 1 eq) and 10% palladium on carbon (400 mg) in MeOH (200 mL) was stirred under an atmosphere of hydrogen at room temperature. After 16 h the reaction was filtered through a pad of Celite and the filtrate concentrated in vacuo to afford the title compound as a black solid (1.74 g, 98%).

¹H NMR (250 MHz, CDCl₃): δ1.88 (m, 2H), 2.17 (m, 2H), 3.19 (q, 2H), 3.91 (br.s, 2H), 6.51 (d, 1H), 6.89 (d, 1H), 7.10 (dd, 1H), 7.19 (s, 1H). LC/MS 244 (MH⁻).

### [5-Cyano-1-(4,4,4-trifluorobutyl)benzimidazol-2-yl]methyl acetate

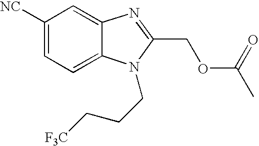

To a stirred solution of 3-amino-4-(4,4,4-trifluorobutylamino)benzonitrile (1.74 g, 7.15 mmol, 1 eq) and triethylamine (1.99 mL, 14.30 mmol, 2 eq) in CH₂Cl₂ (30 mL) at room temperature was added acetoxyacetyl chloride (769 μL, 7.15 mmol, 1 eq) drop wise via syringe. Once the addition was complete the mixture was stirred for a further 10 min. The reaction was washed with water (3×15 mL), dried (MgSO₄), filtered and concentrated in vacuo to yield a dark brown solid. The solid was dissolved in acetic acid (10 mL) and stirred at 80° C. After 16 h the reaction was concentrated in vacuo to dryness and the residue taken up in CH₂Cl₂ (10 mL). The resulting solution was washed with a saturated aqueous solution of Na₂CO₃ (5×5 mL), dried (MgSO₄), filtered and concentrated in vacuo. The residue was purified by reverse phase column chromatography eluting with a 5% through to 80% acetonitrile in water mixture to give the title compound as a pale pink solid (1.05 g, 45%).

¹H NMR (250 MHz, DMSO-d₆): δ1.98 (m, 2H), 2.08 (s, 3H), 2.41 (m, 2H), 4.39 (t, 2H), 5.37 (s, 2H), 7.71 (dd, 1H), 7.89 (dd, 1H), 8.20 (d, 1H). LC/MS 326 (MH⁺).

### 2-(Hydroxymethyl)-1-(4,4,4-trifluorobutyl)benzimidazole-5-carbonitrile

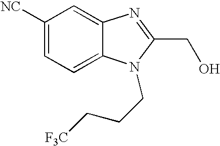

A mixture of [5-cyano-1-(4,4,4-trifluorobutyl)benzimidazol-2-yl]methyl acetate (1.05 g, 3.23 mmol, 1 eq) and K₂CO₃ (895 mg, 6.46 mmol, 2 eq) in MeOH (20 mL) was stirred at room temperature. After 1 h the reaction was concentrated in vacuo to dryness and the residue stirred in a 9:1 CH₂Cl₂:MeOH mixture for 10 min. The mixture was filtered and the filtrate concentrated in vacuo to give an off white solid. Purification by flash column chromatography eluting with 400:8:1 through to 20:8:1 CH₂Cl₂:EtOH:NH₃ yielded the title compound as a white solid (728 mg, 80%).

¹H NMR (250 MHz, DMSO-d₆): δ2.00 (m, 2H), 2.38 (m, 2H), 4.40 (t, 2H), 4.76 (d, 2H), 5.74 (t, 1H), 7.66 (m, 1H), 7.85 (m, 1H), 8.14 (s, 1H). LC/MS 284 (MH⁺).

### [5-(Aminomethyl)-1-(4,4,4-trifluorobutyl)benzimidazol-2-yl]methanol

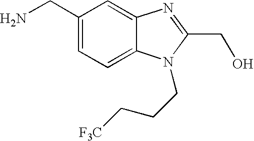

A mixture of 2-(hydroxymethyl)-1-(4,4,4-trifluorobutyl)benzimidazole-5-carbonitrile (822 mg, 2.90 mmol, 1 eq) and palladium hydroxide (170 mg) in a 2:1:0.5 mixture of MeOH:THF:c.HCl (49.6 mL) was shaken at room temperature under an atmosphere of hydrogen. After 16 h the reaction was filtered through a pad of Celite and the filtrate concentrated in vacuo to afford a black gum. The gum was dissolved in water (30 mL) and the solution basified with a saturated aqueous solution of Na₂CO₃. EtOAc was added until a thick precipitate formed. The precipitate was isolated by filtration and kept. The aqueous layer was separated from the filtrate and concentrated in vacuo to dryness. The residue was extracted with 9:1 CH₂Cl₂:MeOH (3×10 mL) and the combined extracts were concentrated in vacuo to dryness to yield an off-white solid. This solid was combined with previously isolated precipitate and dried in vacuo to yield the title compound as an off-white solid of reasonable purity. No further purification was performed.

LC/MS 288 (MH⁺).

### tert-Butyl N-[[2-(hydroxymethyl)-1-(4,4,4-trifluorobutyl)benzimidazol-5-yl]-methyl]carbamate

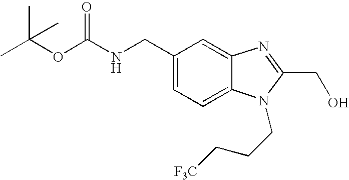

A mixture of [5-(aminomethyl)-1-(4,4,4-trifluorobutyl)benzimidazol-2-yl]methanol (694 mg, 2.42 mmol, 1 eq), di-tert-butyl dicarbonate (528 mg, 2.42 mmol, 1 eq) and diisopropylethylamine (800 μL, 4.84 mmol, 1 eq) in CH₂Cl₂ (10 mL) was stirred at room temperature. After 3 h the reaction was concentrated in vacuo to dryness. The residue was dissolved in MeOH (10 mL) and K₂CO₃ (670 mg, 4.84 mmol, 2 eq) was added. After stirring at room temperature for 1.5 h the reaction was filtered and the filtrate concentrated in vacuo to dryness. The residue was purified by flash column chromatography, eluting with 20:8:1 CH₂Cl₂:EtOH:NH₃, to afford the title compound as a white solid (383 mg, 41%).

¹H NMR (250 MHz, DMSO-d₆): δ1.38 (s, 9H), 2.01 (m, 2H), 2.34 (m, 2H), 4.20 (d, 2H), 4.33 (t, 2H), 4.69 (d, 2H), 5.59 (t, 1H), 7.14 (m, 1H), 7.43 (m, 1H), 7.52 (m, 1H). LC/MS 389 (MH⁺).

### tert-Butyl N-[[2-[(3-cyclopropyl-2-oxo-4H-quinazolin-1-yl)methyl]-1-(4,4,4-trifluoro-butyl)benzimidazol-5-yl]methyl]carbamate

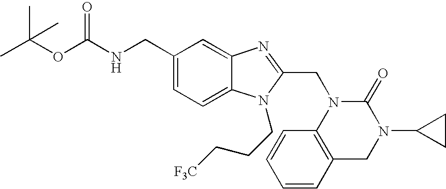

To a stirred solution of tert-butyl N-[[2-(hydroxymethyl)-1-(4,4,4-trifluorobutyl)benzimidazol-5-yl]-methyl]carbamate (2 g, 5.16 mmol, 1 eq) in CH₂Cl₂ (20 mL) at room temperature was added thionyl chloride (753 μL, 10.32 mmol, 2 eq) drop wise via syringe. After 30 min the reaction concentrated in vacuo to dryness to give a yellow-orange residue. The residue was dissolved in DMF (8 mL) and added to a stirred suspension of 3-cyclopropyl-1,4-dihydroquinazolin-2-one (970 mg, 5.16 mmol, 1 eq) and a 60% dispersion of NaH in mineral oil (619 mg, 15.48 mmol, 3 eq) in DMF (12 mL), under an atmosphere of nitrogen. The reaction was then stirred at room temperature for 90 min. The reaction was poured onto water (200 mL) and the resulting suspension was filtered and dried at the pump to give the title compound as an off-white solid (2.82 g, 98%).

¹H NMR (250 MHz, DMSO-d₆): δ0.56 (m, 2H), 0.73 (m, 2H), 1.36 (s, 9H), 1.94 (m, 2H), 2.39 (m, 2H), 2.62 (m, 1H), 4.16 (d, 2H), 4.38 (m, 4H), 5.29 (s, 2H) 6.94 (m, 1H), 7.17 (m, 4H), 7.35 (m, 2H), 7.53 (d, 1H). LC/MS 558 (MH⁺).

### 1-[[5-(Aminomethyl)-1-(4,4,4-trifluorobutyl)benzimidazol-2-yl]methyl]-3-cyclopropyl-4H-quinazolin-2-one (Example 28)

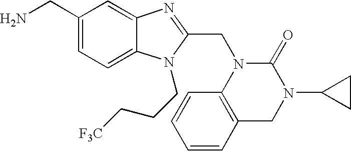

To a stirred solution of tert-butyl N-[[2-[3-cyclopropyl-2-oxo-4H-quinazolin-1-yl)methyl]-1-(4,4,4-trifluoro-butyl)benzimidazol-5-yl]methyl]carbamate (2.82 g, 5.06 mmol, 1 eq) in CH₂Cl₂ (20 mL) at room temperature was added a 4 M solution of HCl in dioxane (10 mL). After 1 h the reaction was concentrated in vacuo to dryness and the residue taken up in water (10 mL). The resulting solution was treated with a saturated aqueous solution of NaHCO₃ in water until no further precipitate was formed. The suspension was filtered and dried at the pump to afford a pale yellow solid. Purification by flash column chromatography, eluting with a gradient of 5-95% 10% MeOH/CH₂Cl₂ in CH₂Cl₂, gave the title compound as a white solid (1.22 g, 53%). ¹H NMR (250 MHz, DMSO-d₆): δ0.58 (m, 2H), 0.76 (m, 2H), 1.99 (m, 2H), 2.41 (m, 2H), 2.64 (m, 1H), 3.77 (s, 2H), 4.39 (m, 4H), 5.30 (s, 2H), 6.97 (m, 1H), 7.21 (m, 4H), 7.51 (m, 2H). LC/MS 458 (MH⁺).

Typical synthesis involving reduction of the corresponding cyano derivative:

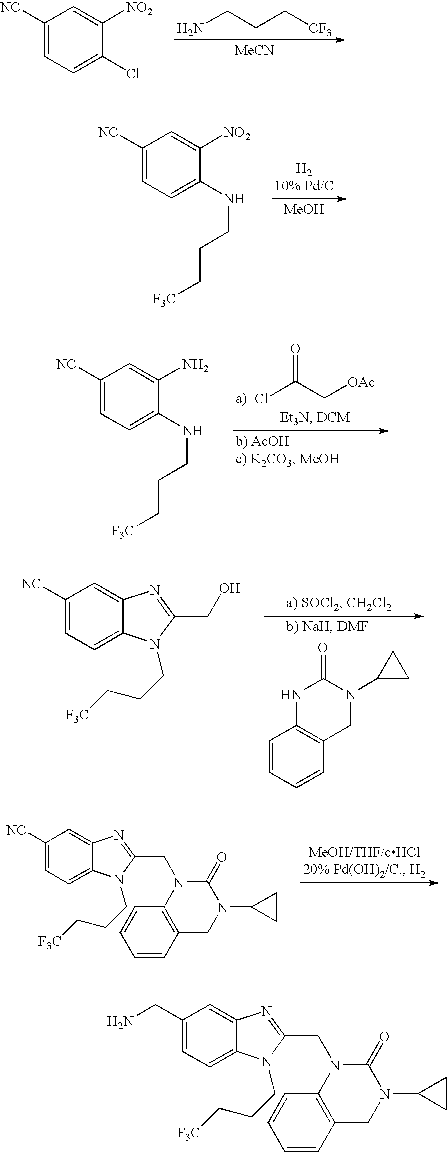

### 2-[(3-Cyclopropyl-2-oxo-4H-quinazolin-1-yl)methyl]-1-(4,4,4-trifluorobutyl)benzimidazole-5-carbonitrile

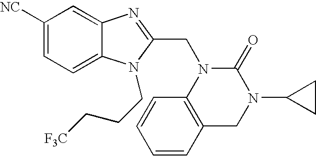

To a stirred solution of 2-(hydroxymethyl)-1-(4,4,4-trifluorobutyl)benzimidazole-5-carbonitrile (2.17 g, 7.66 mmol, 1 eq) in CH₂Cl₂ (50 mL) at room temperature was added thionyl chloride (2.24 mL, 30.64 mmol, 4 eq) drop wise via syringe. After 16 h the reaction concentrated in vacuo to dryness to give an off-white residue. The residue was dissolved in DMF (10 mL) and added over the course of 30 min to a stirred suspension of 3-cyclopropyl-1,4-dihydroquinazolin-2-one (1.44 g, 7.66 mmol, 1 eq) and a 60% dispersion of NaH in mineral oil (919 mg, 22.98 mmol, 3 eq) in DMF (8 mL), under an atmosphere of nitrogen. Water (5 drops) was added followed by concentration in vacuo to dryness. The residue was suspended in a 1:1 mixture of CH₂Cl₂:MeOH (50 mL) and the suspension filtered through Celite. The filtrate was concentrated in vacuo to dryness to afford a yellow gum. Purification of the gum by flash column chromatography, eluting with a gradient of 5-80% MeCN in water yielded the title compound as an off-white solid (2.15 g, 62%). LC/MS 454 (MH⁺).

### 1-[[5-(Aminomethyl)-1-(4,4,4-trifluorobutyl)benzimidazol-2-yl]methyl]-3-cyclopropyl-4H-quinazolin-2-one (Example 28)

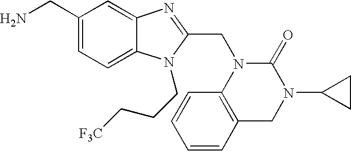

A mixture of 2-[(3-Cyclopropyl-2-oxo-4H-quinazolin-1-yl)methyl]-1-(4,4,4-trifluoro-butyl)benzimidazole-5-carbonitrile (2.15 g, 4.73 mmol, 1 eq) and 20% Pd(OH)₂/C (200 mg) in MeOH (30 mL), THF (15 mL), and c. HCl (1 mL) was shaken under an atmosphere of hydrogen. After 16 h the reaction mixture was filtered over Celite and the filtrate concentrated in vacuo to dryness to give an off-white residue. The residue was purified by flash column chromatography, eluting with a gradient of 20-70% 20:8:1 CH₂Cl₂:MeOH:NH₄OH in CH₂Cl₂, to yield the title compound as a white solid (1.79 g, 83%).

¹H NMR (250 MHz, DMSO-d₆): δ0.58 (m, 2H), 0.76 (m, 2H), 1.99 (m, 2H), 2.41 (m, 2H), 2.64 (m, 1H), 3.77 (s, 2H), 4.39 (m, 4H), 5.30 (s, 2H), 6.97 (m, 1H), 7.21 (m, 4H), 7.51 (m, 2H). LC/MS 458 (MH⁺).

### EXAMPLE 28(I)

Larger Scale Preparation of 1-[[5-(Aminomethyl)-1-(4,4,4-trifluorobutyl)benzimidazol-2-yl]methyl]-3-cyclopropyl-4H-quinazolin-2-one

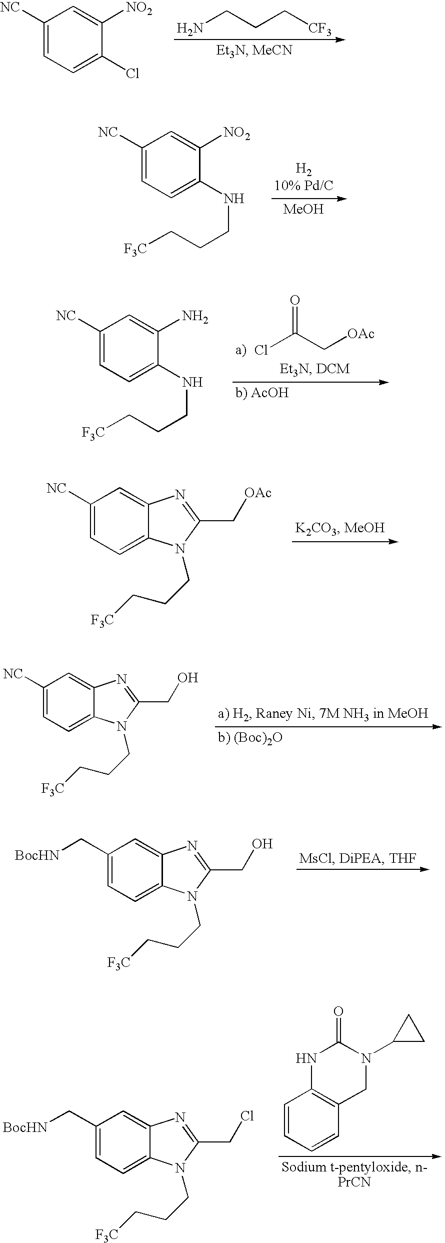

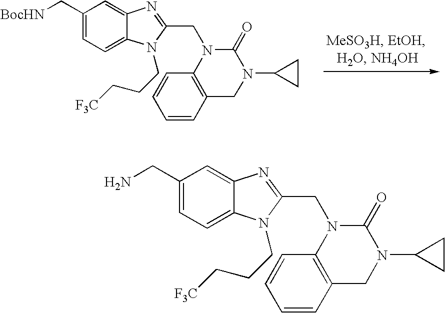

### 3-Nitro-4-(4,4,4-trifluorobutylamino)benzonitrile

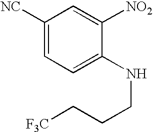

Under an atmosphere of nitrogen, a 5 L flask equipped with an overhead stirrer was charged 4-chloro-3-nitro benzonitrile (114.92 g, 0.63 mol, 1 eq), 4,4,4-trifluorobutylamine (80 g, 0.63 mol, 1 eq), MeCN (2.4 L) and Et₃N (87.8 mL, 0.63 mol, 1 eq) at room temperature. The reaction was then heated at 70° C. After 16 h ˜7% of the starting material remained by LC and further 4,4,4-trifluorobutylamine (3.8 g, 0.03 mol, 0.05 eq) and NEt₃ (4 mL, 0.05 eq) were charged and the reaction was stirred at 70° C. for a further 1.5 h. The reaction mixture was cooled to room temperature and concentrated in vacuo to dryness. The residue was slurried in water (3 L) for 1.5 h, filtered, and pulled dry to afford the title compound (163.4 g, 95%).

¹H NMR (250 MHz, CDCl₃): δ2.10 (m, 2H), 2.33 (m, 2H), 3.51 (q, 2H), 6.95 (d, 1H), 7.68 (m, 1H), 8.44 (br.s, 1H), 8.57 (d, 1H)

### 3-Amino-4-(4,4,4-trifluorobutylamino)benzonitrile

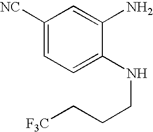

To a 5 L flask equipped with an overhead stirrer was charged a suspension of 10% Pd—C (16 g) in MeOH (2 L). Under an atmosphere of nitrogen, 3-nitro-4-(4,4,4-trifluorobutylamino)benzonitrile (160.3 g, 0.58 mol) in MeOH (1.2 L) was then charged at room temperature. Hydrogen gas was then purged through the suspension for 4.5 h. The reaction mixture was filtered through Celite and concentrated in vacuo to dryness to afford the title compound (135.2 g, 95%).

¹H NMR (250 MHz, CDCl₃): δ1.88 (m, 2H), 2.17 (m, 2H), 3.19 (q, 2H), 3.91 (br.s, 2H), 6.51 (d, 1H), 6.89 (d, 1H), 7.10 (dd, 1H), 7.19 (s, 1H).

### [5-Cyano-1-(4,4,4-trifluorobutyl)benzimidazol-2-yl]methyl acetate

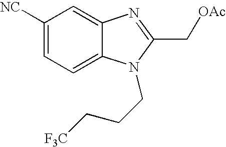

Under an atmosphere of nitrogen, a 2 L flask equipped with an overhead stirrer was charged 3-amino-4-(4,4,4-trifluorobutylamino)benzonitrile (134 g, 0.55 mol, 1 eq) followed by CH₂Cl₂ (2 L) at room temperature. The solution was cooled in an ice bath to 5-10° C. and Et₃N (153 mL, 1.1 mol, 2 eq) was charged (no exotherm). Acetoxyacetyl chloride (59 mL, 0.55 mol, 1 eq) was then added drop wise whilst the pot temperature was maintained <15° C. The reaction mixture was then allowed to warm up to room temperature and stirred. After 16 h further acetoxyacetyl chloride (6 mL, 0.055, 0.1 eq) was added and stirred for 1 h. The reaction mixture was concentrated in vacuo to dryness and the residue taken up in AcOH (1 L). The mixture was heated to 80° C. for 18 h and cooled to ˜45-50° C. AcOH was distilled under vacuum and the residue was taken up in CH₂Cl₂ (1.35 L). The CH₂Cl₂ layer was washed with a saturated aqueous solution of NaHCO₃ (3×800 mL), dried (MgSO₄), and concentrated in vacuo to dryness affording the title compound (185 g, >100%).

¹H NMR (250 MHz, DMSO-d₆): δ1.98 (m, 2H), 2.08 (s, 3H), 2.41 (m, 2H), 4.39 (t, 2H), 5.37 (s, 2H), 7.71 (dd, 1H), 7.89 (dd, 1H), 8.20 (d, 1H).

### 2-(Hydroxymethyl)-1-(4,4,4-trifluorobutyl)benzimidazole-5-carbonitrile

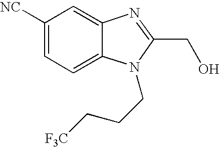

Under an atmosphere of nitrogen, a 10 L flask equipped with a overhead stirrer was charged with [5-cyano-1-(4,4,4-trifluorobutyl)benzimidazol-2-yl]methyl acetate (183 g, 0.56 mol, 1 eq) in MeOH (3 L) followed by Na₂CO₃ (119.4 g, 1.13 mol, 2 eq) at room temperature. After stirring at room temperature for 1 h the reaction was diluted with water (3 L) and the pH was adjusted to 7 using AcOH (140 mL). The reaction was further diluted with water (3 L) and stirred for 30 min. The precipitated product was filtered, washed with water (1.5 L) and dried under vacuum at 45° C. with a bleed for 40 h to afford the title compound (112.3 g, 71%).

¹H NMR (250 MHz, DMSO-d₆): δ2.00 (m, 2H), 2.38 (m, 2H), 4.40 (t, 2H), 4.76 (d, 2H), 5.74 (t, 1H), 7.66 (m, 1H), 7.85 (m, 1H), 8.14 (s, 1H). LC/MS 284 (MH⁺).

### tert-Butyl N-[[2-(hydroxymethyl)-1-(4,4,4-trifluorobutyl)benzimidazol-5-yl]-methyl]carbamate

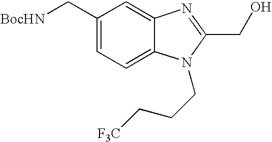

To a 7 L hydrogenator was charged a suspension of Raney-Ni (58 g, wet, most of the water decanted) in 7 M NH₃ in MeOH (400 mL). 2-(Hydroxymethyl)-1-(4,4,4-trifluorobutyl)benzimidazole-5-carbonitrile (120 g, 0.42 mol, 1 eq) was then charged as a solution in 7 M NH₃ in MeOH (4 L) and stirred at 1000 rpm. The reaction was purged with nitrogen (3×5 bar), hydrogen (3×5 bar) and filled with hydrogen (5 bar). The reaction was continued overnight (hydrogen absorption was topped up) at room temperature and 5 bar of hydrogen. The reaction mixture was filtered through an in-line cuno filter and washed with 7 M NH₃ in MeOH (500 mL). The filtrate was concentrated in vacuo to half the volume and transferred to a 10 L flask. Di-tert-butyldicarbonate (93.4 mL, 0.42 mol, 1 eq) was added drop wise over a period of 20 min (pot temp<16° C.) and the mixture stirred at room temperature under an inert atmosphere of nitrogen overnight. A further amount of Di-tert-butyldicarbonate was charged to push the reaction to completion (a total of 41.8 mL over a period of 7 h). 10% Sodium metabisulfite (250 g in 2.5 L water) was charged to the reaction mixture and the suspension stirred at room temperature overnight. The mixture was filtered and the residue washed with water (1 L) and dried under vacuum at 40° C. for 20 h. The dried solid was slurried in hot toluene (245.5 mL) for 30 min and then the slurry was allowed to cool to room temperature. Filtration, washing with MTBE (196 mL), and drying under vacuum for 18 h afforded the title compound (86.7 g, 52%).

¹H NMR (250 MHz, DMSO-d₆): δ1.38 (s, 9H), 2.01 (m, 2H), 2.34 (m, 2H), 4.20 (d, 2H), 4.33 (t, 2H), 4.69 (d, 2H), 5.59 (t, 1H), 7.14 (m, 1H), 7.43 (m, 1H), 7.52 (m, 1H).

### tert-butyl N-[[2-(chloromethyl)-1-(4,4,4-trifluorobutyl)benzimidazol-5-yl]ethyl]carbamate

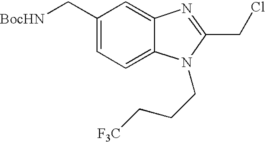

Under an inert atmosphere of nitrogen, a 5 L flask was charged tert-butyl N-[[2-(hydroxymethyl)-1-(4,4,4-trifluorobutyl)-benzimidazol-5-yl]-methyl]carbamate (79 g, 0.20 mol, 1 eq) followed by THF (790 mL) at room temperature. The solution was cooled to 0-5° C. and DIPEA (105.9 mL, 0.61 mol, 3 eq) was charged followed by drop wise addition of MsCl (20.6 mL, 0.27 mol, 1.3 eq). After stirring at room temperature for 16 h EtOAc (790 mL) was charged followed by water (790 mL). The biphasic mixture was stirred 15 min and the layers were separated. The aqueous layer was extracted with EtOAc (2×400 mL). The combined organic layers were washed with a 10% aqueous solution of citric acid (400 mL), a saturated aqueous solution of NaHCO₃ (400 mL), water (400 mL), brine (400 mL) and dried (MgSO₄). Following filtration the solvent was reduced in vacuo to dryness to afford the title compound 85.8 g, 104%).

### tert-Butyl N-[[2-[(3-cyclopropyl-2-oxo-4H-quinazolin-1-yl)methyl]-1-(4,4,4-trifluoro-butyl)benzimidazol-5-yl]methyl]carbamate

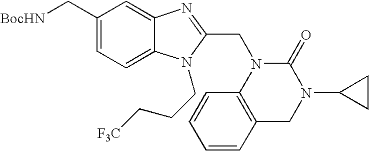

Under an inert atmosphere of nitrogen, a 500 mL flask under was charged with 3-cyclopropyl-1,4-dihydroquinazolin-2-one (37.57 g, 0.20 mol, 1 eq) followed by butyronitrile (289 mL). To this suspension was added sodium tert-pentyloxide (21.98 g, 0.21 mol, 1.05 eq) in portions. The thick suspension was warmed to 40° C. and stirred until a clear solution formed (˜45 min). The solution was then cooled to 30° C. and held at this temperature. Under an inert atmosphere of nitrogen, a separate 1 L flask was charged with tert-butyl N-[[2-(chloromethyl)-1-(4,4,4-trifluoro-butyl)benzimidazol-5-yl]ethyl]carbamate (81 g, 0.20 mol, 1 eq) and then butyronitrile (289 mL) at room temperature. The solution was heated to 55° C. and the clear 3-cyclopropyl-1,4-dihydroquinazolin-2-one in butyronitrile solution was added drop wise while maintaining the pot temperature at 55-60° C. (˜45 min). After 30 min sodium tert-pentyloxide (1.09 g, 0.01 mol, 0.02 eq) in butyronitrile (25 mL) was added to the reaction mixture. After 30 min water (289 mL) containing AcOH (12 mL) was added and the reaction mixture heated at 90° C. for 15 min. The reaction was cooled to 80° C. and the layers separated. The organic layer was charged to a 2 L flask and heptane (1156 mL) was added and stirred overnight at room temperature. The reaction mixture was cooled to 0-5° C. for 2 h and filtered after the addition of further heptane (250 mL). The residue was dissolved in EtOAc (2 L) and passed through a pad of Celite (100 g) and silica (150 g). The pad was rinsed with EtOAc (8 L). The EtOAc was removed in vacuo and heptane (500 mL) was charged to the residue and slurried for 15 min. The product was filtered and pulled dry under vacuum to afford the title compound (79.1 g, 71%).

¹H NMR (250 MHz, DMSO-d₆): δ0.56 (m, 2H), 0.73 (m, 2H), 1.36 (s, 9H), 1.94 (m, 2H), 2.39 (m, 2H), 2.62 (m, 1H), 4.16 (d, 2H), 4.38 (m, 4H), 5.29 (s, 2H) 6.94 (m, 1H), 7.17 (m, 4H), 7.35 (m, 2H), 7.53 (d, 1H).

### 1-[[5-(Aminomethyl)-1-(4,4,4-trifluorobutyl)benzimidazol-2-yl]methyl]-3-cyclopropyl-4H-quinazolin-2-one

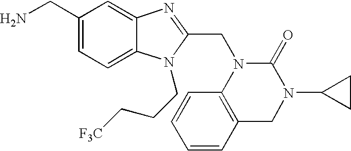

A 1 L flask was charged with tert-butyl N-[[2-[(3-cyclopropyl-2-oxo-4H-quinazolin-1-yl)methyl]-1-(4,4,4-trifluoro-butyl)benzimidazol-5-yl]methyl]carbamate (78.5 g, 0.14 mol, 1 eq) followed by EtOH (337.5 mL) and water (188.4 mL) at room temperature. The suspension was stirred and heated to 55° C. MeSO₃H (22.8 mL, 0.35 mol, 2.5 eq) was added drop wise whilst maintaining the pot temp 55-60° C. Water (39.25 mL) was then added to the clear solution thus formed and continued to be stirred at 55° C. After 16 h charcoal (2 g) was added to the solution and stirred for another 30 min at 55° C. The reaction mixture was filtered hot through a pad of Celite (50 g) and the pad was washed with warm 1:1 EtOH:water (75 mL). The filtrate was transferred to another 1 L flask and warmed to 40° C. Aqueous NH₃ solution (98.9 mL) in water (75 mL) was added drop wise while maintaining the pot temp<45° C. Water (37.5 mL) was added and stirred for 2 h at 40° C. The reaction mixture was cooled to 20° C. and water (150 mL) was added drop wise over a period of 30 min. The suspension was further cooled to 10-15° C. and filtered after stirring for 30 min. The residue was washed with water (2×150 mL) and pulled dry The solid was transferred to a 5 L flask and 20% MeOH in water (2.5 L) was charged followed by a seed crystal. The suspension was stirred at 250 rpm under nitrogen at room temperature for 16 h and filtered. The product was washed with water (250 mL) and dried under vacuum at 24° C. with bleed for 48 h to afford the title compound (59 g, 92%). ¹H NMR (250 MHz, DMSO-d₆): δ0.58 (m, 2H), 0.76 (m, 2H), 1.99 (m, 2H), 2.41 (m, 2H), 2.64 (m, 1H), 3.77 (s, 2H), 4.39 (m, 4H), 5.30 (s, 2H), 6.97 (m, 1H), 7.21 (m, 4H), 7.51 (m, 2H).

The product obtained in Example 28(i) was analysed by XRPD. The X-ray powder diffraction spectra showed the material to be crystalline and the material was designated as 1-[[5-(aminomethyl)-1-(4,4,4-trifluorobutyl)benzimidazol-2-yl]methyl]-3-cyclopropyl-4H-quinazolin-2-one Form A.

1-[[5-(Aminomethyl)-1-(4,4,4-trifluorobutyl)benzimidazol-2-yl]methyl]-3-cyclopropyl-4H-quinazolin-2-one Form A is characterised by providing an X-ray powder diffraction pattern, substantially as shown in FIG. 21A. The seven most prominent peaks are shown in Table 21:

TABLE 21
Seven most Prominent X-Ray Powder Diffraction peaks for
1-[[5-(Aminomethyl)-1-(4,4,4-trifluorobutyl)benzimidazol-2-
yl]methyl]-3-cyclopropyl-4H-quinazolin-2-one Form A

| Angle 2- | | Relative |
|---|---|---|
| Theta (2θ) | Intensity % | Intensity |
| 7.685 | 100.0 | vs |
| 21.154 | 89.5 | vs |
| 21.896 | 88.9 | vs |
| 20.107 | 84.4 | vs |
| 22.785 | 72.6 | vs |
| 15.032 | 68.1 | vs |
| 17.447 | 62.6 | vs |

vs = very strong

DSC analysis of 1-[[5-(aminomethyl)-1-(4,4,4-trifluorobutyl)benzimidazol-2-yl]methyl]-3-cyclopropyl-4H-quinazolin-2-one Form A shows an initial event with an onset at 56.9° C. and a peak at 71.7° C. followed by an exothermic event, followed by a subsequent melting endotherm with an onset of 155.5° C. and a peak at 158.3° C. (FIG. 21B). DSC analysis shows 1-[[5-(aminomethyl)-1-(4,4,4-trifluorobutyl)benzimidazol-2-yl]methyl]-3-cyclopropyl-4H-quinazolin-2-one Form A is a high melting solid with an onset of melting at about 155.5° C. and a peak at about 158.3° C.

### EXAMPLE 28(II)

### Preparation of 1-[[5-(Aminomethyl)-1-(4,4,4-trifluorobutyl)benzimidazol-2-yl]methyl]-3-cyclopropyl-4H-quinazolin-2-one Form B

The X-ray powder diffraction spectra for 1-[[5-(aminomethyl)-1-(4,4,4-trifluorobutyl)benzimidazol-2-yl]methyl]-3-cyclopropyl-4H-quinazolin-2-one Form A showed the material to be crystalline. This material had a melting point of 155.5° C. (onset).

Form B material was produced by slurrying the 1-[[5-(aminomethyl)-1-(4,4,4-trifluorobutyl)benzimidazol-2-yl]methyl]-3-cyclopropyl-4H-quinazolin-2-one Form A material in aqueous methanol (aqueous methanol consists of approximately 20% to 30% methanol in water). Approximately 20 mg of the 1-[[5-(aminomethyl)-1-(4,4,4-trifluorobutyl)benzimidazol-2-yl]methyl]-3-cyclopropyl-4H-quinazolin-2-one Form A material was placed in a vial with a magnetic flea, and approximately 2 ml of aqueous methanol added. The vial was then sealed tightly with a cap and left to stir on a magnetic stirrer plate. After 3 days, the sample was removed from the plate, the cap taken off and the slurry left to dry under ambient conditions before it was analysed by XRPD and DSC. This form (1-[[5-(Aminomethyl)-1-(4,4,4-trifluorobutyl)benzimidazol-2-yl]methyl]-3-cyclopropyl-4H-quinazolin-2-one Form C) was determined to be crystalline by XRPD and had a melting point of 154.0° C. (onset).

1-[[5-(Aminomethyl)-1-(4,4,4-trifluorobutyl)benzimidazol-2-yl]methyl]-3-cyclopropyl-4H-quinazolin-2-one Form B is characterised by providing an X-ray powder diffraction pattern, substantially as shown in FIG. 22A. The ten most prominent peaks are shown in Table 22:

TABLE 22
Ten most Prominent X-Ray Powder Diffraction peaks for
1-[[5-(Aminomethyl)-1-(4,4,4-trifluorobutyl)benzimidazol-2-
yl]methyl]-3-cyclopropyl-4H-quinazolin-2-one Form B

| Angle 2- | | Relative |
|---|---|---|
| Theta (2θ) | Intensity % | Intensity |
| 17.063 | 100.0 | vs |
| 7.838 | 97.7 | vs |
| 8.300 | 90.7 | vs |
| 20.136 | 72.8 | vs |
| 22.791 | 70.9 | vs |
| 21.065 | 65.1 | vs |
| 10.391 | 44.9 | vs |
| 13.392 | 41.6 | vs |
| 12.992 | 40.9 | vs |
| 12.567 | 32.8 | vs |

vs = very strong

DSC analysis of 1-[[5-(Aminomethyl)-1-(4,4,4-trifluorobutyl)benzimidazol-2-yl]methyl]-3-cyclopropyl-4H-quinazolin-2-one Form B shows an initial event with an onset at 50.2° C. and a peak at 88.2° C. followed by a subsequent melting endotherm with an onset of 154.0° C. and a peak at 156.7° C. (FIG. 22B). DSC analysis shows 1-[[5-(Aminomethyl)-1-(4,4,4-trifluorobutyl)benzimidazol-2-yl]methyl]-3-cyclopropyl-4H-quinazolin-2-one Form B is a high melting solid with an onset of melting at about 154.0° C. and a peak at about 156.7° C.

### EXAMPLE 28(III)

### Preparation of 1-[[5-(Aminomethyl)-1-(4,4,4-trifluorobutyl)benzimidazol-2-yl]methyl]-3-cyclopropyl-4H-quinazolin-2-one Form C

The X-ray powder diffraction spectra for 1-[[5-(aminomethyl)-1-(4,4,4-trifluorobutyl)benzimidazol-2-yl]methyl]-3-cyclopropyl-4H-quinazolin-2-one Form A showed the material to be crystalline. This material had a melting point of 155.5° C. (onset).

1-[[5-(Aminomethyl)-1-(4,4,4-trifluorobutyl)benzimidazol-2-yl]methyl]-3-cyclopropyl-4H-quinazolin-2-one Form C material was produced by slurrying the Form A material in water. Approximately 20 mg of the 1-[[5-(aminomethyl)-1-(4,4,4-trifluorobutyl)benzimidazol-2-yl]methyl]-3-cyclopropyl-4H-quinazolin-2-one Form A material was placed in a vial with a magnetic flea, and approximately 2 ml of water added. The vial was then sealed tightly with a cap and left to stir on a magnetic stirrer plate. After 3 days, the sample was removed from the plate, the cap taken off and the slurry left to dry under ambient conditions before it was analysed by XRPD and DSC. This form (1-[[5-(aminomethyl)-1-(4,4,4-trifluorobutyl)benzimidazol-2-yl]methyl]-3-cyclopropyl-4H-quinazolin-2-one Form C) was determined to be crystalline by XRPD and had a melting point of 154.2° C. (onset).

1-[[5-(Aminomethyl)-1-(4,4,4-trifluorobutyl)benzimidazol-2-yl]methyl]-3-cyclopropyl-4H-quinazolin-2-one Form C is characterised by providing an X-ray powder diffraction pattern, substantially as shown in FIG. 23A. The seven most prominent peaks are shown in Table 23:

TABLE 23
Seven most Prominent X-Ray Powder Diffraction peaks for
1-[[5-(Aminomethyl)-1-(4,4,4-trifluorobutyl)benzimidazol-2-
yl]methyl]-3-cyclopropyl-4H-quinazolin-2-one Form C

| Angle 2- | | Relative |
|---|---|---|
| Theta (2θ) | Intensity % | Intensity |
| 7.812 | 55.2 | vs |
| 21.166 | 49.8 | vs |
| 20.135 | 42.0 | vs |
| 8.343 | 40.2 | vs |
| 15.004 | 32.2 | vs |
| 23.498 | 31.6 | vs |
| 25.709 | 31.5 | vs |

vs = very strong

DSC analysis of 1-[[5-(aminomethyl)-1-(4,4,4-trifluorobutyl)benzimidazol-2-yl]methyl]-3-cyclopropyl-4H-quinazolin-2-one Form C shows an initial event with an onset at 53.1° C. and a peak at 89.3° C. followed by a subsequent melting endotherm with an onset of 154.2° C. and a peak at 158.2° C. (FIG. 23B). DSC analysis shows 1-[[5-(Aminomethyl)-1-(4,4,4-trifluorobutyl)benzimidazol-2-yl]methyl]-3-cyclopropyl-4H-quinazolin-2-one Form C is a high melting solid with an onset of melting at about 154.2° C. and a peak at about 158.2° C.

### EXAMPLE 28(IV)

### Preparation of 1-[[5-(Aminomethyl)-1-(4,4,4-trifluorobutyl)benzimidazol-2-yl]methyl]-3-cyclopropyl-4H-quinazolin-2-one IPA solvate

The X-ray powder diffraction spectra for 1-[[5-(aminomethyl)-1-(4,4,4-trifluorobutyl)benzimidazol-2-yl]methyl]-3-cyclopropyl-4H-quinazolin-2-one Form A showed the material to be crystalline. This material had a melting point of 155.5° C. (onset).

1-[[5-(aminomethyl)-1-(4,4,4-trifluorobutyl)benzimidazol-2-yl]methyl]-3-cyclopropyl-4H-quinazolin-2-one IPA solvate was produced by slurrying the 1-[[5-(aminomethyl)-1-(4,4,4-trifluorobutyl)benzimidazol-2-yl]methyl]-3-cyclopropyl-4H-quinazolin-2-one Form A material in isopropyl alcohol (IPA). Approximately 20 mg of the 1-[[5-(aminomethyl)-1-(4,4,4-trifluorobutyl)benzimidazol-2-yl]methyl]-3-cyclopropyl-4H-quinazolin-2-one Form A material was placed in a vial with a magnetic flea, and approximately 2 ml of IPA added. The vial was then sealed tightly with a cap and left to stir on a magnetic stirrer plate. After 3 days, the sample was removed from the plate, the cap taken off and the slurry left to dry under ambient conditions before it was analysed by XRPD and DSC. This form was determined to be crystalline by XRPD and had a melting point of 149.5° C. (onset).

1-[[5-(Aminomethyl)-1-(4,4,4-trifluorobutyl)benzimidazol-2-yl]methyl]-3-cyclopropyl-4H-quinazolin-2-one IPA solvate is characterised by providing an X-ray powder diffraction pattern, substantially as shown in FIG. 24A. The ten most prominent peaks are shown in Table 24:

TABLE 24
Ten most Prominent X-Ray Powder Diffraction peaks for
1-[[5-(aminomethyl)-1-(4,4,4-trifluorobutyl)benzimidazol-2-
yl]methyl]-3-cyclopropyl-4H-quinazolin-2-one IPA solvate

| Angle 2- | | Relative |
|---|---|---|
| Theta (2θ) | Intensity % | Intensity |
| 9.410 | 100.0 | vs |
| 7.507 | 93.4 | vs |
| 16.815 | 79.0 | vs |
| 6.638 | 64.5 | vs |
| 22.589 | 62.1 | vs |
| 20.213 | 48.8 | vs |
| 4.278 | 45.9 | vs |
| 24.095 | 45.4 | vs |
| 18.932 | 44.8 | vs |
| 8.527 | 43.5 | vs |

vs = very strong

DSC analysis of 1-[[5-(aminomethyl)-1-(4,4,4-trifluorobutyl)benzimidazol-2-yl]methyl]-3-cyclopropyl-4H-quinazolin-2-one IPA solvate shows an initial event with an onset at 53.1° C. and a peak at 67.8° C., followed by a further endotherm with an onset at 112.9° C. and a peak at 123.5° C., followed by a subsequent melt with an onset of 149.5° C. and a peak at 154.8° C. (FIG. 24B). DSC analysis shows 1-[[5-(aminomethyl)-1-(4,4,4-trifluorobutyl)benzimidazol-2-yl]methyl]-3-cyclopropyl-4H-quinazolin-2-one IPA solvate is a high melting solid with an onset of melting at about 149.5° C. and a peak at about 154.8° C.

### EXAMPLE 28(V)

### Preparation of 1-[[5-(Aminomethyl)-1-(4,4,4-trifluorobutyl)benzimidazol-2-yl]methyl]-3-cyclopropyl-4H-quinazolin-2-one acetonitrile solvate

The X-ray powder diffraction spectra for 1-[[5-(aminomethyl)-1-(4,4,4-trifluorobutyl)benzimidazol-2-yl]methyl]-3-cyclopropyl-4H-quinazolin-2-one Form A showed the material to be crystalline. This material had a melting point of 155.5° C. (onset).

1-[[5-(Aminomethyl)-1-(4,4,4-trifluorobutyl)benzimidazol-2-yl]methyl]-3-cyclopropyl-4H-quinazolin-2-one acetonitrile solvate was produced by slurrying the 1-[[5-(aminomethyl)-1-(4,4,4-trifluorobutyl)benzimidazol-2-yl]methyl]-3-cyclopropyl-4H-quinazolin-2-one Form A material in acetonitrile. Approximately 20 mg of the 1-[[5-(aminomethyl)-1-(4,4,4-trifluorobutyl)benzimidazol-2-yl]methyl]-3-cyclopropyl-4H-quinazolin-2-one Form A material was placed in a vial with a magnetic flea, and approximately 2 ml of acetonitrile added. The vial was then sealed tightly with a cap and left to stir on a magnetic stirrer plate. After 3 days, the sample was removed from the plate, the cap taken off and the slurry left to dry under ambient conditions before it was analysed by XRPD and DSC. This form 1-[[5-(aminomethyl)-1-(4,4,4-trifluorobutyl)benzimidazol-2-yl]methyl]-3-cyclopropyl-4H-quinazolin-2-one acetonitrile solvate was determined to be crystalline by XRPD and had a melting point of 126.6° C. (onset).

1-[[5-(Aminomethyl)-1-(4,4,4-trifluorobutyl)benzimidazol-2-yl]methyl]-3-cyclopropyl-4H-quinazolin-2-one acetonitrile solvate is characterised by providing an X-ray powder diffraction pattern, substantially as shown in FIG. 25A. The nine most prominent peaks are shown in Table 25:

TABLE 25
Nine most Prominent X-Ray Powder Diffraction peaks for
1-[[5-(aminomethyl)-1-(4,4,4-trifluorobutyl)benzimidazol-2-
yl]methyl]-3-cyclopropyl-4H-quinazolin-2-one acetonitrile solvate

| Angle 2- | | Relative |
|---|---|---|
| Theta (2θ) | Intensity % | Intensity |
| 21.580 | 90.1 | vs |
| 23.512 | 77.1 | vs |
| 6.976 | 73.8 | vs |
| 19.541 | 71.4 | vs |
| 9.537 | 69.8 | vs |
| 17.108 | 67.7 | vs |
| 9.008 | 59.7 | vs |
| 7.524 | 55.1 | vs |
| 13.035 | 53.1 | vs |

vs = very strong

DSC analysis of 1-[[5-(aminomethyl)-1-(4,4,4-trifluorobutyl)benzimidazol-2-yl]methyl]-3-cyclopropyl-4H-quinazolin-2-one acetonitrile solvate shows an initial event with an onset at 34.9° C. and a peak at 58.1° C. followed by a subsequent melt with an onset of 126.6° C. and a peak at 146.1° C. (FIG. 25B). DSC analysis shows 1-[[5-(aminomethyl)-1-(4,4,4-trifluorobutyl)benzimidazol-2-yl]methyl]-3-cyclopropyl-4H-quinazolin-2-one acetonitrile solvate is a high melting solid with an onset of melting at about 126.6° C. and a peak at about 146.1° C.

### EXAMPLE 28(VI)

### Preparation of 1-[[5-(Aminomethyl)-1-(4,4,4-trifluorobutyl)benzimidazol-2-yl]methyl]-3-cyclopropyl-4H-quinazolin-2-one methanol solvate

The X-ray powder diffraction spectra for 1-[[5-(aminomethyl)-1-(4,4,4-trifluorobutyl)benzimidazol-2-yl]methyl]-3-cyclopropyl-4H-quinazolin-2-one Form A showed the material to be crystalline. This material had a melting point of 155.5° C. (onset).

1-[[5-(aminomethyl)-1-(4,4,4-trifluorobutyl)benzimidazol-2-yl]methyl]-3-cyclopropyl-4H-quinazolin-2-one methanol solvate was produced by slurrying the 1-[[5-(aminomethyl)-1-(4,4,4-trifluorobutyl)benzimidazol-2-yl]methyl]-3-cyclopropyl-4H-quinazolin-2-one Form A material in methanol. Approximately 20 mg of the 1-[[5-(aminomethyl)-1-(4,4,4-trifluorobutyl)benzimidazol-2-yl]methyl]-3-cyclopropyl-4H-quinazolin-2-one Form A material was placed in a vial with a magnetic flea, and approximately 2 ml of methanol added. The vial was then sealed tightly with a cap and left to stir on a magnetic stirrer plate. After 3 days, the sample was removed from the plate, the cap taken off and the slurry left to dry under ambient conditions before it was analysed by XRPD and DSC. This form 1-[[5-(aminomethyl)-1-(4,4,4-trifluorobutyl)benzimidazol-2-yl]methyl]-3-cyclopropyl-4H-quinazolin-2-one methanol solvate was determined to be crystalline by XRPD and had a melting point of 148.3° C. (onset).

1-[[5-(Aminomethyl)-1-(4,4,4-trifluorobutyl)benzimidazol-2-yl]methyl]-3-cyclopropyl-4H-quinazolin-2-one methanol solvate is characterised by providing an X-ray powder diffraction pattern, substantially as shown in FIG. 26A. The four most prominent peaks are shown in Table 26A:

TABLE 26
Four most Prominent X-Ray Powder Diffraction peaks for
1-[[5-(aminomethyl)-1-(4,4,4-trifluorobutyl)benzimidazol-2-
yl]methyl]-3-cyclopropyl-4H-quinazolin-2-one methanol solvate

| Angle 2- | | Relative |
|---|---|---|
| Theta (2θ) | Intensity % | Intensity |
| 4.787 | 100.0 | vs |
| 9.548 | 65.3 | vs |
| 12.629 | 46.7 | vs |
| 8.283 | 31.0 | vs |

vs = very strong

DSC analysis of 1-[[5-(aminomethyl)-1-(4,4,4-trifluorobutyl)benzimidazol-2-yl]methyl]-3-cyclopropyl-4H-quinazolin-2-one methanol solvate shows an initial event with an onset at 37.4° C. and a peak at 69.6° C. followed by an exothermic event followed by a subsequent melt with an onset of 148.3° C. and a peak at 152.4° C. (FIG. 26B). DSC analysis shows 1-[[5-(aminomethyl)-1-(4,4,4-trifluorobutyl)benzimidazol-2-yl]methyl]-3-cyclopropyl-4H-quinazolin-2-one methanol solvate is a high melting solid with an onset of melting at about 148.3° C. and a peak at about 152.4° C.

### EXAMPLES 31 TO 33

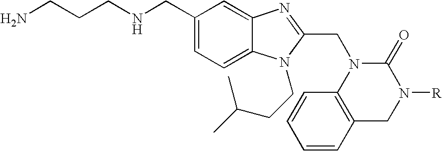

| Ex. | R | ¹H NMR | LC/MS |
|---|---|---|---|
| 31 | Me | (250 MHz, DMSO-d₆): δ 0.97(6H, d), 1.65(5H, m), 2.77(2H, t), 2.94(3H, s), 3.78(2H, t), 4.26(2H, t), 4.44(2H, s), 5.32(2H, s), 7.07(4H, m), 7.49(3H, m), 8.30(2H, br.s) | 450 (MH⁺) |
| 32 | Cyclopropyl | (250 MHz, DMSO-d₆): δ 0.54(m, 2H), 0.74(m, 2H), 0.96(d, 6H), 1.55(m, 2H), 1.69(m, 3H), 2.60(t, 2H), 2.80(t, 1H), 3.83(s, 2H), 4.27(t, 2H), 4.39(s, 2H), 4.48(s, 2H), 5.32(s, 2H), 6.91(m, 1H), 7.13 (m, 4H), 7.44(d, 1H), 7.52(s, 1H), 8.33(s, 2H) | 476 (MH⁺) |
| 33 | 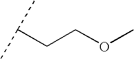 | (250 MHz, DMSO-d₆): δ 0.97(d, 6H), 1.65(m, 5H), 2.66(m, 2H), 2.81(t, 1H), 3.26(s, 3H), 3.58(s, 2H), 3.77(s, 2H), 4.30(m, 4H), 4.52(s, 2H), 5.65(s, 2H), 6.92(m, 1H), 7.20(m, 4H), 7.50(m, 2H), 8.30(bs, 2H) | 494 (MH⁺) |

These compounds were prepared according to the following scheme:

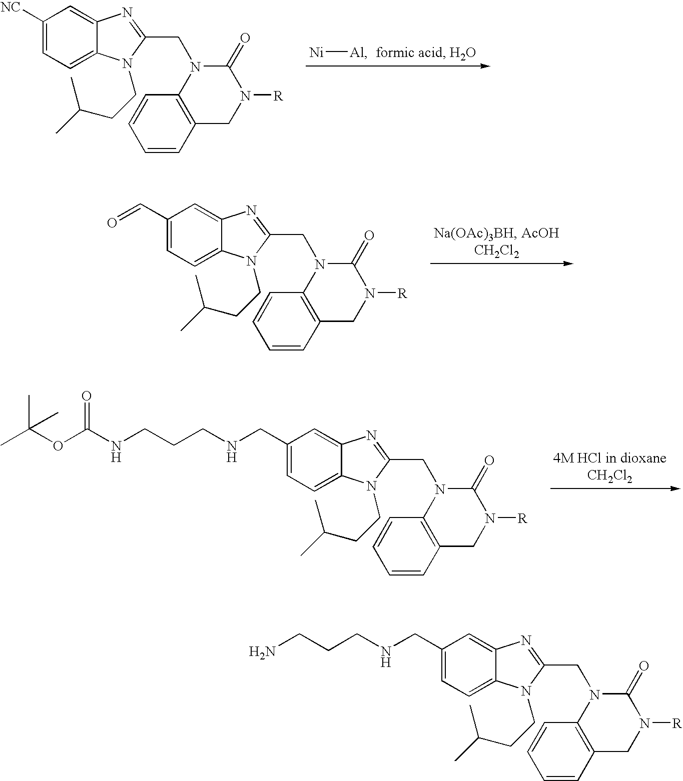

Starting materials for Examples 31 to 33:

### tert-Butyl N-[3-[[1-isopentyl-2-[(3-substituted-2-oxo-4H-quinazolin-1-yl)methyl]benzimidazol-5-yl]methylamino]propyl]carbamates:

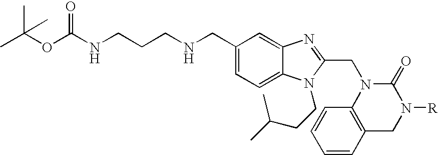

General Procedure:

To a stirred solution of a benzimidazole-5-carbaldehyde (1 eq) in CH₂Cl₂ at room temperature was added acetic acid (a few drops) followed by tert-butyl-N-(3-aminopropyl)carbamate (1.5 eq). After 1 h sodium triacetoxyborohydride was added. After a further 16 h at room temperature the reaction was quenched with a small amount of triethylamine. The reaction was then concentrated in vacuo to dryness to yield an off-white solid. The solid was used in the next step without further purification.

| R | LC/MS |
|---|---|
| Me | 550 (MH⁺) |
| Cyclopropyl | 576 (MH⁺) |
| 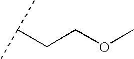 | 594 (MH⁺) |

### 1-Isopentyl-2-[(3-substituted-2-oxo-4H-quinazolin-1-yl)methyl]benzimidazole-5-carbaldehydes:

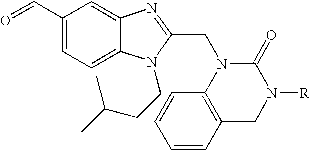

General Procedure:

To a stirred solution of a benzimidazole-5-carbonitrile in a 2:1 mixture of formic acid and water was added Ni—Al (Raney type). The resulting suspension was heated at 100° C. After 30 min the reaction was allowed to cool to room temperature and then concentrated in vacuo to dryness. The residue was then purified by column chromatography, eluting with 40:8:1 CH₂Cl₂:EtOH:NH₃, to give the title compound as an off-white solid.

| R | LC/MS |
|---|---|
| Me | 392 (MH⁺) |
| Cyclopropyl | 418 (MH⁺) |
| 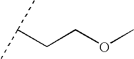 | 436 (MH⁺) |

### 1-Isopentyl-2-[(3-substituted-2-oxo-4H-quinazolin-1-yl)methyl]benzimidazole-5-carbonitriles:

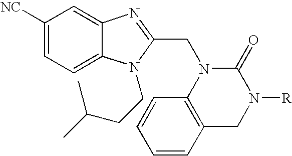

The benzimidazole-5-carbonitriles were prepared as those for Examples 5 to 23.

| R | MS Data |
|---|---|
| Me | 389 (MH⁺) |
| Cyclopropyl | 415 (MH⁺) |
| 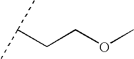 | 433 (MH⁺) |

### EXAMPLE 34

### 1-[[5-[(3-Aminopropylamino)methyl]-1-(4-hydroxybutyl)benzimidazole-2-yl]methyl]-3-methyl-4H-quinazolin-2-one

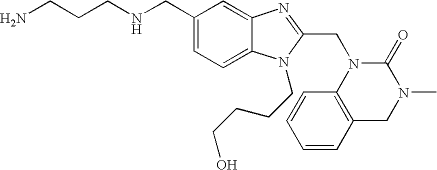

A mixture of 4-[5-[(3-aminopropylamino)methyl]-2-[(3-methyl-2-oxo-4H-quinazolin-1-yl)methyl]benzimidazol-1-yl]butyl 2,2-dimethylpropanoate (160 mg, 0.3 mmol, 1 eq) and K₂CO₃ (166 mg, 1.2 mmol, 4 eq) in MeOH was stirred at room temperature. After 16 h the reaction was filtered through a pad of Celite and the filtrate concentrated in vacuo to dryness. The residue was purified by HPLC to yield the title compound as a white solid (62 mg, 46%).

¹H NMR (250 MHz, DMSO-d₆): δ1.51 (2H, m), 1.78 (4H, m), 2.85 (2H, t), 2.97 (3H, s), 3.42 (2H, t), 3.58 (2H, t), 3.78 (2H, s), 4.30 (2H, t), 4.48 (2H, s), 5.34 (2H, s), 6.97 (1H, m), 7.19 (4H, m), 7.50 (1H, s), 8.32 (1H, br.s). LC/MS 452 (MH⁺).

The starting materials for Example 34 were prepared in the same manner as Examples 31 to 33 but substituting the appropriate benzimidazole derivative.

### EXAMPLES 35 TO 40

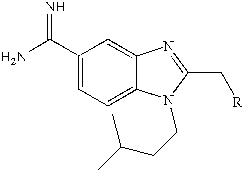

| Ex | R | ¹H NMR | LC/MS |
|---|---|---|---|
| 35 | 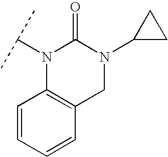 | 1H NMR (250 MHz, DMSO-d₆): δ 0.55(m, 2H), 0.74(m, 2H), 0.97(d, 6H), 1.60-1.70(m, 3H), 2.62 (m, 1H), 4.35(t, 2H), 4.40(s, 2H), 5.36(s, 2H), 6.94 (m, 1H), 7.06(d, 1H), 7.13-7.21(m, 2H), 7.66(s, 2H), 8.01(s, 1H), 9.16(br.s, 3H) | 431 (MH⁺) |
| 36? | 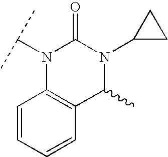 | 1H NMR (250 MHz, DMSO-d₆): δ 0.49(m, 1H), 0.55(m, 1H), 0.67(m, 1H), 0.86(m, 1H), 0.97(d, 6H), 1.70(m, 3H), 2.67(m, 1H), 4.38(m, 2H), 4.55 (m, 1H), 5.41(q, 2H), 6.98(m, 1H), 7.04(m, 1H), 7.11(m, 1H), 7.18(m, 1H), 7.66(m, 1H), 7.76(d, 1H), 8.05(d, 1H), 8.41(s, 1H), 8.82(br.s, H), 11.29(br.s, 1H) | 445 (MH⁺) |
| 37 | 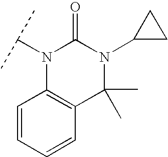 | 1H NMR (250 MHz, DMSO-d₆): δ 0.46(m, 2H), 0.82(m, 2H), 0.98(d, 6H), 1.64(m, 9H), 2.38(m, 1H), 4.39(t, 3H), 5.41(s, 2H), 6.98(m, 1H), 7.04 (m, 1H), 7.16(m, 1H), 7.29(m, 1H), 7.65(m, 2H), 7.94(m, 1H), 8.84(br.s, 3H) | 459( MH⁺) |

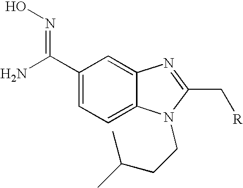

| Ex | R | ¹H NMR | LC/MS |
|---|---|---|---|
| 38 | 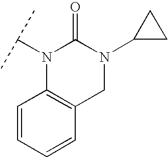 | ¹H NMR (250 MHz, DMSO-d₆): δ 0.58(m, 2H), 0.72(m, 2H), 0.97(d, 6H), 1.61(m, 2H), 1.67(m, 1H), 2.62(m, 1H), 4.29(t, 2H), 4.40(s, 2H), 5.33(s, 2H), 5.76(s, 2H), 6.94(td, 1H), 7.12(m, 1H), 7.20, (m, 2H), 7.47(d, 1H), 7.59(dd, 1H), 7.82(d, 1H), 9.49(s, 1H) | 431 (MH⁺) |
| 39 | 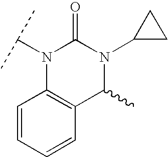 | 1H NMR (250 MHz, DMSO-d₆): δ 0.57(m, 2H), 0.73(m, 2H), 1.29(d, 3H), 1.54(m, 3H), 2.68(m, 1H), 4.22(m, 2H), 4.46(m, 1H), 5.27(dd, 2H), 6.86 (t, 1H), 7.08(m, 1H), 7.18(m, 2H), 7.41(m, 1H), 7.50(d, 1H), 7.70(m, 1H), 8.08(m, 1H), 9.39(br.s, 1H) | 461 (MH⁺) |
| 40 | 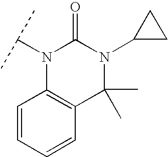 | 1H NMR (250 MHz, DMSO-d₆): δ 0.49(m, 2H), 0.81(m, 2H), 0.98(d, 6H), 1.64(m, 9H), 2.37(m, 1H), 4.34(t, 2H), 5.37(s, 2H), 6.99(t, 1H), 7.09(d, 1H), 7.18(t, 1H), 7.31(d, 1H), 7.48(d, 1H), 7.58 (dd, 1H), 7.76(s, 1H), 8.14(s, 1H), 9.46(br.s, 1H) | 475 (MH⁺) |

Examples 35 to 40 were prepared using a procedure analogous to that described in WO 03/053344 (page 14; Scheme V). The process involved the coupling of 2-(chloromethyl)-1-isopentyl-benzimidazole-5-carbonitrile, HCl salt, (WO 03/053344; page 27) with the indicated quinazolinone. The carbonitrile product was converted to a N-hydroxycarboxamidine, which in turn was converted to a carboxamidine:

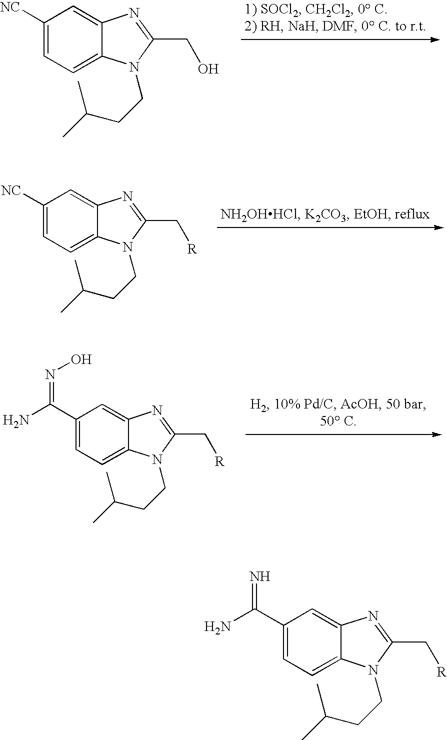

General Procedure:

To a stirred solution of 2-(hydroxymethyl)-1-isopentyl-benzimidazole-5-carbonitrile (1 eq) in CH₂Cl₂ at 0° C. was added thionyl chloride (2 eq) drop wise. After 30 min the reaction was reduced in vacuo to dryness. The residue was dissolved in DMF and the resulting solution was added to a stirred mixture of the appropriate quinazolinone (1 eq) and a 60% dispersion of NaH in mineral oil (3 eq) in DMF, under an atmosphere of nitrogen. After 45 min a few drops of water were added and the mixture was reduced in vacuo to dryness. The residue was purified by flash column chromatography, eluting with 20:8:1 CH₂Cl₂:EtOH:NH₃, to yield a 2-[(3-cyclopropylquinazolin-1-yl)methyl]-1-isopentyl-benzimidazole-5-carbonitrile HCl salt as a white solid.

A mixture of the 2-[(3-cyclopropylquinazolin-1-yl)methyl]-1-isopentyl-benzimidazole-5-carbonitrile HCl salt (1 eq), hydroxylamine hydrochloride (3 eq) and K₂CO₃ (1.5 eq) in EtOH was heated at reflux. After 16 h the reaction was allowed to cool to room temperature and reduced in vacuo. The residue was dissolved in CH₂Cl₂ and washed with water, dried (MgSO₄), filtered and concentrated in vacuo to dryness to give a white solid. The solid was purified by flash column chromatography, eluting with 20:8:1 CH₂Cl₂:EtOH:NH₃, to give a 2-[(3-cyclopropylquinazolin-1-yl)methyl]-N′-hydroxy-1-isopentyl-benzimidazole-5-carboxamidine as a white solid

A solution of the 2-[(3-cyclopropylquinazolin-1-yl)methyl]-N′-hydroxy-1-isopentyl-benzimidazole-5-carboxamidine (1 eq) in AcOH was hydrogenated in the presence of 10% Pd/C at 50 bar and 50° C. using a H-cube apparatus. The reaction mixture was concentrated in vacuo to dryness. The residue was purified by flash column chromatography, eluting with 20:8:1 CH₂Cl₂:EtOH:NH₃, to give a 2-[(3-cyclopropylquinazolin-1-yl)methyl]-1-isopentyl-benzimidazole-5-carboxamidine as a white solid.

### EXAMPLES 41 TO 45

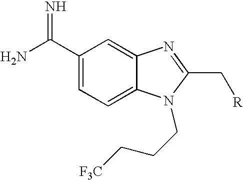

| # | R | ¹H NMR | LC/MS |
|---|---|---|---|
| 41 | 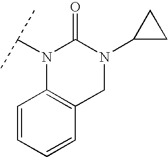 | 1H NMR (250 MHz, DMSO-d₆): δ 0.62(m, 2H), 0.82 (m, 2H), 2.08(m, 2H), 2.67(m, 1H), 4.47(s, 2H), 4.54(t, 2H), 5.43(s, 2H), 7.03(t, 1H), 7.15(t, 1H), 7.23(m, 1H), 7.28(m, 1H), 7.77(dd, 1H), 7.93(d, 1H), 8.14(d, 1H), 8.53(s, 1H) | 471 MH⁺) |
| 42 | 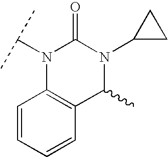 | 1H NMR (250 MHz, DMSO-d₆): δ 0.47(m, 1H), 0.56 (m, 1H), 0.69(m, 1H), 0.85(m, 1H), 1.38(d, 3H), 2.02(m, 2H), 2.42(m, 2H), 2.67(m, 1H), 4.46(m, 2H), 4.52(m, 1H), 5.37(m, 2H), 6.96(m, 1H), 7.12 (m, 1H), 7.16(m, 1H), 7.20(m, 1H), 7.69(m, 1H), 7.77(m, 1H), 7.98(d, 1H), 8.95(br.s, 3H) | 485 (MH⁺) |
| 43 | 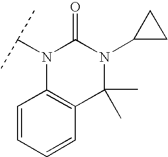 | 1H NMR (250 MHz, DMSO-d₆): δ 0.46(m, 2H), 0.81 (m, 2H), 1.64(s, 6H), 2.03(m, 2H), 2.37(m, 2H), 2.47(m, 1H), 4.48(t, 2H), 5.39(s, 2H), 7.01(m, 1H), 7.10(m, 1H), 7.19(m, 1H), 7.32(m, 1H), 7.70 (m, 1H), 7.75(m, 1H), 7.97(m, 1H), 8.20(br.s, 3H) | 499 (MH⁺) |

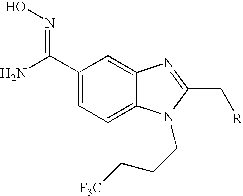

| # | R | ¹H NMR | LC/MS |
|---|---|---|---|
| 44 | 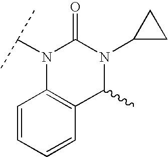 | 1H NMR (250 MHz, DMSO-d6): δ 0.48(m, 1H), 0.55(m, 1H), 0.67(m, 1H), 0.85(m, 1H), 1.37(d, 3H), 2.03(m, 2H), 2.45(m, 2H), 2.67(m, 1H), 4.42 (m, 2H), 4.52(m, 1H), 5.33(m, 2H), 6.94(m, 1H), 7.18(m, 3H), 7.57(m, 1H), 7.79(m, 2H), 7.93(s, 1H), 9.46(br.s, 1H) | 501 (MH⁺) |
| 45 | 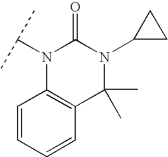 | 1H NMR (250 MHz, DMSO-d₆): δ 0.38(m, 2H), 0.72 (m, 2H), 1.56(s, 6H), 1.95(m, 2H), 2.31(m, 3H), 4.36(t, 2H), 5.28(s, 2H), 6.93(m, 1H), 7.08(m, 2H), 7.24(m, 1H), 7.51(m, 2H), 7.70(m, 1H), 8.12 (s, 1H), 9.40(br.s, 1H) | 515 (MH⁺) |

Examples 41 to 45 were prepared in the same manner as Examples 35 to 40 but substituting the appropriate benzimidazole derivative.

### EXAMPLE 46

### 1-[[5-(Aminomethyl)-1-isopentyl-6-methyl-benzimidazol-2-yl]methyl]-3-cyclopropyl-4H-quinazolin-2-one

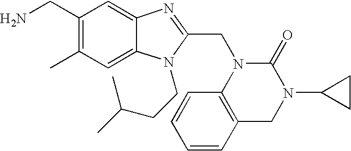

The title compound was prepared in an analogous manner to Examples 5 to 23 but substituting the appropriate fluoronitrobenzonitrile.

¹H NMR (250 MHz, DMSO-d₆): δ0.56 (m, 2H), 0.77 (m, 2H), 0.96 (d, 6H), 1.54 (m, 2H), 1.68 (m, 1H), 2.36 (s, 3H), 2.62 (m, 1H), 3.71 (s, 2H), 4.22 (t, 2H), 4.38 (s, 2H), 5.30 (s, 2H), 6.92 (m, 1H), 7.15 (m, 3H), 7.22 (m, 1H), 7.47 (s, 1H). LC/MS 432 (MH⁺).

### 4-Fluoro-2-methyl-5-nitro-benzonitrile

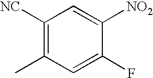

Prepared according to the procedure described in WO 2007/036718 (page 85).

¹H NMR (250 MHz, CDCl₃): δ2.60 (s, 3H), 7.23 (d, 1H), 8.30 (d, 1H).

### 4-(Isopentylamino)-2-methyl-5-nitro-benzonitrile

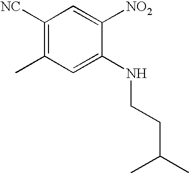

¹H NMR (250 MHz, CDCl₃): δ0.93 (d, 6H), 1.59 (m, 2H), 1.71 (m, 1H), 2.49 (s, 3H), 3.27 (m, 2H), 6.64 (s, 1H), 8.24 (br.s, 1H), 8.39 (s, 1H). LC/MS 248 (MH).

### 5-Amino-4-(isopentylamino)-2-methyl-benzonitrile

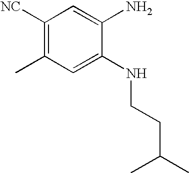

¹H NMR (250 MHz, CDCl₃): δ0.89 (d, 6H), 1.50 (m, 2H), 1.68 (m, 1H), 2.35 (s, 3H), 3.03-3.11 (m, 4H), 3.89 (br.s, 1H), 6.35 (s, 1H), 6.80 (s, 1H). LC/MS 217 (MH⁺).

### 2-(Hydroxymethyl)-1-isopentyl-6-methyl-benzimidazole-5-carbonitrile

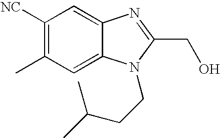

¹H NMR (250 MHz, DMSO-d₆): δ0.94 (d, 6H), 1.58-1.69 (m, 3H), 2.58 (s, 3H), 4.27 (t, 2H), 4.71 (d, 2H), 5.71 (t, 1H), 7.61 (s, 1H), 8.04 (s, 1H). LC/MS 258 (MH⁺).

### [5-(Aminomethyl)-1-isopentyl-6-methyl-benzimidazol-2-yl]methanol

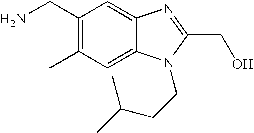

¹H NMR (250 MHz, DMSO-d₆): δ0.96 (d, 6H), 1.68 (m, 3H), 2.54 (s, 3H), 4.16 (m, 2H), 4.37 (t, 2H), 5.02 (s, 2H), 7.82 (s, 1H), 7.88 (s, 1H), 8.68 (br.s, 2H). LC/MS 262 (MH⁺).

### tert-Butyl N-[[2-(hydroxymethyl)-1-isopentyl-6-methyl-benzimidazol-5-yl]-methyl]carbamate

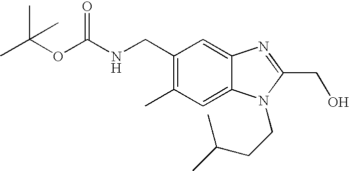

¹H NMR (250 MHz, DMSO-d₆): δ0.93 (d, 6H), 1.40 (s, 9H), 1.61 (m, 2H), 1.74 (m, 1H), 2.36 (s, 2H), 3.59 (m, 1H), 4.14-4.24 (m, 4H))⁾ 4.65 (d, 2H), 5.54 (t, 1H), 7.25 (s, 1H), 7.37 (s, 1H). LC/MS 362 (MH⁻).

### tert-Butyl N-[[2-[(3-cyclopropyl-2-oxo-4H-quinazolin-1-yl)methyl]-1-isopentyl-6-methyl-benzimidazol-5-yl]methyl]carbamate

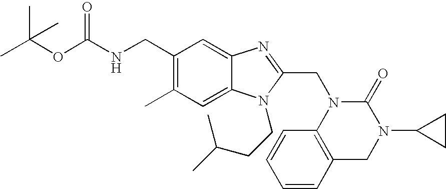

LC/MS 532 (MH⁺).

Pharmacological Example

The antiviral activity of these compounds against RSV was determined in an ELISA assay. In such an assay, reduction in RSV protein expression demonstrates a compound's ability to interrupt the RSV life cycle.

ELISA assays were set up by seeding 96-well plates with 5×10³ Hep-2 cells per well in 100 μl DMEM containing 10% FBS for 24 h. Compound dilutions were prepared in 0.5% DMSO and 50 μl/well was added to the assay plates, the plates were incubated at 37° C. in 5% CO₂ for one hour. Cells were infected with 50 μl/well RSV RSS strain at a multiplicity of infection of 0.02 and were incubated at 37° C. in 5% CO₂ for 3 days. Cells were then fixed and permeabilised with 75%/25% methanol acetone and blocked with 2% Marvel™, 0.05% Tween for one hour. Plates were then incubated in the presence of a 1:4000 dilution of anti-goat RSV polyclonal antibody (Millipore, AB1128) followed by a 1:1000 dilution of rabbit anti-goat, horseradish peroxidase (DAKO, PO449) labelled secondary antibody. The plates were developed with O-phenylenediamine in the presence of hydrogen peroxide.

The antiviral activity of the compounds is presented as an IC₅₀, the concentration required to inhibit RSV protein expression by 50%.

When tested in the above screen, all of the compounds of the Examples gave IC₅₀ values of less than 10 μM (micromolar), indicating that the compounds of the invention are expected to possess useful therapeutic properties. Specimen results are shown in the following Table:

TABLE

| Example No. | ELISA IC50 (μM) |
|---|---|
| 1 | 1.89 |
| 2 | >1 |
| 3 | 1.3 |
| 4 | 1.42 |
| 5 | 0.0089 |
| 6 | 0.0012 |
| 7 | 0.0017 |
| 8 | 0.0167 |
| 9 | 0.0013 |
| 10 | 0.0157 |
| 11 | 0.0188 |
| 12 | 0.0531 |
| 13 | 0.1013 |
| 14 | 0.0021 |
| 15 | 0.0019 |
| 16 | 0.0059 |
| 17 | 0.0137 |
| 18 | 0.0040 |
| 19 | 2.886 |
| 20 | 0.0094 |
| 21 | 0.0570 |
| 22 | 0.0089 |
| 23 | 0.0077 |
| 24 | 0.0032 |
| 25 | 0.0015 |
| 26 | 0.0004 |
| 27 | 0.0940 |
| 28 | 0.0005 |
| 29 | 0.0038 |
| 30 | 0.0110 |
| 31 | 0.0230 |
| 32 | 0.0320 |
| 33 | 0.2430 |
| 34 | 0.0840 |
| 35 | 0.0032 |
| 36 | 0.0084 |
| 37 | 0.0420 |
| 38 | 0.0889 |
| 39 | 0.3996 |
| 40 | >1.0 |
| 41 | 0.0016 |
| 42 | 0.0118 |
| 43 | 0.0180 |
| 44 | 0.2291 |
| 45 | >1.0 |
| 46 | 0.0015 |

Thermodynamic Solubility

Assay Protocol:

A known amount of the test compound was stirred in 0.1M phosphate buffer pH 7.4 at constant temperature (25° C.) for 24 h. The supernatant was then separated from un-dissolved material by double centrifugation, and subsequently analysed and quantified against a standard of known concentration in DMSO using generic HPLC-UV methodology coupled with mass spectral peak identification. Specimen results for quinazolin-2-one derivatives of the present invention are shown in the following Table. Data are also included for a reference quinazolin-2,4-dione derivative disclosed in WO 03/053344.

| Compound | Thermodynamic Solubility (μM) |
|---|---|
| Example 5 | 2290 |
| Example 7 | 2300 |
| Example 13 | 1560 |
| Example 20 | 654 |
| Example 21 | 1050 |
| Example 23 | 242 |
| Example 28 | 289 |
| Example 35 | 808 |
| Example 46 | 831 |
| 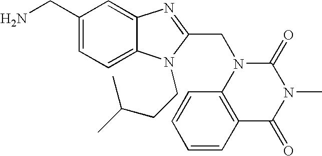 | 16 |
| 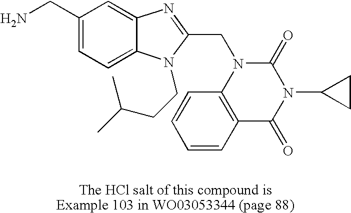 | 70 |
| 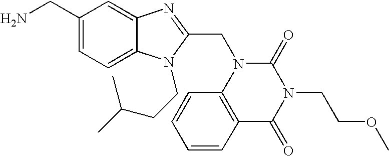 | 36 |
| 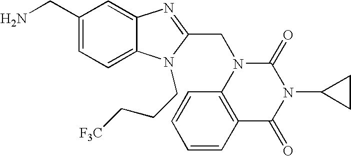 | 90 |

1. A compound of formula (I), or a pharmaceutically acceptable salt thereof, 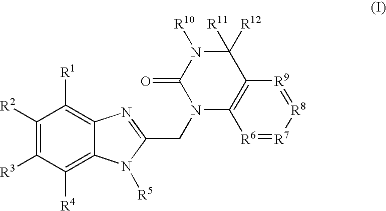
wherein
R¹, R³ and R⁴ each independently represents H, C₁₋₆ alkyl or halogen;
R² represents H, CN, CH₂NH₂, CH₂NH(CH₂)₃NH₂, C(═NH)NH₂ or C(═NOH)NH₂;
R⁵ represents C₁₋₆ alkyl; said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ independently represent H, C₁₋₆ alkyl or C3-7 cycloalkyl; or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl;
R⁶, R⁷, R⁸ and R⁹ each independently represents CH, C—F, C—Cl, C—CF₃ or N;
R¹⁰ represents aryl, heteroaryl, C3-7 cycloalkyl or C₁₋₆ alkyl; said C₁₋₆ alkyl or C3-7 cycloalkyl being optionally substituted with one or more of aryl, C3-7 cycloalkyl, OR¹⁶, SR¹⁶, halogen or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷ R¹⁸ together represents a 5 to 7 membered azacyclic ring optionally incorporating one further heteroatom selected from O, S and NR²⁰ wherein R²⁰ represents H or C1-6 alkyl; and
R¹¹ and R¹² each independently represents H or C1-6 alkyl.

2. A compound according to claim 1, or a pharmaceutically acceptable salt thereof,
wherein
R¹, R³ and R⁴ each independently represents H, C₁₋₆ alkyl or halogen;
R² represents H, CN, CH₂NH₂, CH₂NH(CH₂)₃NH₂, C(═NH)NH₂ or C(═NOH)NH₂;
R⁵ represents C₁₋₆ alkyl; said C₁₋₆ alkyl being optionally substituted with one or more of OR¹³, CF₃, CN or NR¹⁴R¹⁵ wherein R¹³ represents H or C₁₋₆ alkyl and R¹⁴ and R¹⁵ independently represent H, C₁₋₆ alkyl or C3-7 cycloalkyl; or the group —NR¹⁴R¹⁵ together represents a 5 to 7 membered azacyclic ring optionally incorporating one further heteroatom selected from O, S and NR¹⁹ wherein R¹⁹ represents H or C1-6 alkyl;
R⁶, R⁷, R⁸ and R⁹ each independently represents CH, C—F, C—Cl or N;
R¹⁰ represents aryl, heteroaryl, C3-7 cycloalkyl or C₁₋₆ alkyl; said C₁₋₆ alkyl being optionally substituted with one or more of aryl, C3-7 cycloalkyl, OR¹⁶, SR¹⁶, halogen or NR¹⁷R¹⁸, wherein R¹⁶ represents H or C₁₋₆ alkyl and R¹⁷ and R¹⁸ each independently represents H, C₁₋₆ alkyl or C3-7 cycloalkyl; or the group —NR¹⁷R¹⁸ together represents a 5 to 7 membered azacyclic ring optionally incorporating one further heteroatom selected from O, S and NR²⁰ wherein R²⁰ represents H or C1-6 alkyl; and
R¹¹ and R¹² each independently represents H or C1-6 alkyl.

3. A compound according to claim 1, or a pharmaceutically acceptable salt thereof, wherein R² represents CH₂NH₂.

4. A compound according to claim 1, or a pharmaceutically acceptable salt thereof, wherein R⁵ represents C1-6 alkyl optionally substituted with one or more of OH or CF₃, particularly CF₃.

5. A compound according to claim 1, or a pharmaceutically acceptable salt thereof, wherein R¹⁰ represents C3-7 cycloalkyl or C1-6 alkyl; said C₁₋₆ alkyl being optionally substituted with one or more of aryl, C3-7 cycloalkyl, OR¹⁶, SR¹⁶, halogen or NR¹⁷R¹⁸, particularly with one or more of aryl, C3-7 cycloalkyl, OR¹⁶, SR¹⁶ or NR¹⁷R¹⁸.

6. A compound according to claim 5, or a pharmaceutically acceptable salt thereof, wherein R¹⁰ represents C3-7 cycloalkyl, particularly cyclopropyl.

7. A compound according to claim 1, or a pharmaceutically acceptable salt thereof, wherein R¹¹ and R¹² each independently represents H or methyl.

8. A compound according to claim 7, or a pharmaceutically acceptable salt thereof, wherein R¹¹ and R¹² each represents H.

9. A compound according to claim 1 selected from:
3-methyl-1-[(1-isopentylbenzimidazol-2-yl)methyl]-4H-quinazolin-2-one;
3-isopentyl-1-[(1-isopentylbenzimidazol-2-yl)methyl]-4H-quinazolin-2-one;
3-cyclopropyl-1-[(1-isopentylbenzimidazol-2-yl)methyl]-4-methyl-4H-quinazolin-2-one;
3-cyclopropyl-1-[(1-isopentylbenzimidazol-2-yl)methyl]-4,4-dimethyl-quinazolin-2-one;
1-[[5-(aminomethyl)-1-isopentyl-benzimidazol-2-yl]methyl]-3-methyl-4H-quinazolin-2-one;
1-[[5-(aminomethyl)-1-isopentyl-benzimidazol-2-yl]methyl]-3-propyl-4H-quinazolin-2-one;
1-[[5-(aminomethyl)-1-isopentyl-benzimidazol-2-yl]methyl]-3-cyclopropyl-4H-quinazolin-2-one;
1-[[5-(aminomethyl)-1-isopentyl-benzimidazol-2-yl]methyl]-3-tert-butyl-4H-quinazolin-2-one;
1-[[5-(aminomethyl)-1-isopentyl-benzimidazol-2-yl]methyl]-3-cyclopentyl-4H-quinazolin-2-one;
1-[[5-(aminomethyl)-1-isopentyl-benzimidazol-2-yl]methyl]-3-benzyl-4H-quinazolin-2-one;
1-[[5-(aminomethyl)-1-isopentyl-benzimidazol-2-yl]methyl]-3-phenethyl-4H-quinazolin-2-one;
1-[[5-(aminomethyl)-1-isopentyl-benzimidazol-2-yl]methyl]-3-cyclopropyl-4H-pyrido[2,3-d]pyrimidin-2-one;
1-[[5-(aminomethyl)-1-isopentyl-benzimidazol-2-yl]methyl]-3-(2-methoxyethyl)-4H-quinazolin-2-one;
1-[[5-(aminomethyl)-1-isopentyl-benzimidazol-2-yl]methyl]-3-isopentyl-4H-quinazolin-2-one;
1-[[5-(aminomethyl)-1-isopentyl-benzimidazol-2-yl]methyl]-3-isobutyl-4H-quinazolin-2-one;
1-[[5-(aminomethyl)-1-isopentyl-benzimidazol-2-yl]methyl]-3-(cyclopropylmethyl)-4H-quinazolin-2-one;
1-[[5-(aminomethyl)-1-isopentyl-benzimidazol-2-yl]methyl]-3-(3-pyrrolidin-1-ylpropyl)-4H-quinazolin-2-one;
1-[[5-(aminomethyl)-1-isopentyl-benzimidazol-2-yl]methyl]-3-(2-methylsulfanylethyl)-4H-quinazolin-2-one;
1-[[5-(aminomethyl)-1-isopentyl-benzimidazol-2-yl]methyl]-3-(cyclohexylmethyl)-4H-quinazolin-2-one;
1-[[5-(aminomethyl)-1-isopentyl-benzimidazol-2-yl]methyl]-3-cyclopropyl-4-methyl-4H-quinazolin-2-one;
1-[[5-(aminomethyl)-1-isopentyl-benzimidazol-2-yl]methyl]-3-cyclopropyl-4,4-dimethyl-quinazolin-2-one;
1-[[5-(aminomethyl)-1-isopentyl-benzimidazol-2-yl]methyl]-3-cyclopropyl-5-(trifluoromethyl)-4H-quinazolin-2-one;
1-[[5-(aminomethyl)-1-isopentyl-benzimidazol-2-yl]methyl]-3-cyclopropyl-5-fluoro-4H-quinazolin-2-one;
1-[[5-(aminomethyl)-1-(4-hydroxybutyl)benzimidazol-2-yl]methyl]-3-methyl-4H-quinazolin-2-one;
1-[[5-(aminomethyl)-1-(4-hydroxybutyl)benzimidazol-2-yl]methyl]-3-cyclopropyl-4H-quinazolin-2-one;
1-[[5-(aminomethyl)-1-(4-hydroxybutyl)benzimidazol-2-yl]methyl]-3-(2-methoxyethyl)-4H-quinazolin-2-one;
1-[[5-(aminomethyl)-1-(4-hydroxybutyl)benzimidazol-2-yl]methyl]-3-(cyclohexylmethyl)-4H-quinazolin-2-one;
1-[[5-(aminomethyl)-1-(4,4,4-trifluorobutyl)benzimidazol-2-yl]methyl]-3-cyclopropyl-4H-quinazolin-2-one;
1-[[5-(aminomethyl)-1-(4,4,4-trifluorobutyl)benzimidazol-2-yl]methyl]-3-cyclopropyl-4-methyl-4H-quinazolin-2-one;
1-[[5-(aminomethyl)-1-(4,4,4-trifluorobutyl)benzimidazol-2-yl]methyl]-3-cyclopropyl-4,4-dimethyl-quinazolin-2-one;
1-[[5-[(3-aminopropylamino)methyl]-1-isopentyl-benzimidazol-2-yl]methyl]-3-methyl-4H-quinazolin-2-one;
1-[[5-[(3-aminopropylamino)methyl]-1-isopentyl-benzimidazol-2-yl]methyl]-3-cyclopropyl-4H-quinazolin-2-one;
1-[[5-[(3-aminopropylamino)methyl]-1-isopentyl-benzimidazol-2-yl]methyl]-3-(2-methoxyethyl)-4H-quinazolin-2-one;
1-[[5-[(3-aminopropylamino)methyl]-1-(4-hydroxybutyl)benzimidazol-2-yl]methyl]-3-methyl-4H-quinazolin-2-one;
2-[(3-cyclopropyl-2-oxo-4H-quinazolin-1-yl)methyl]-1-isopentyl-benzimidazole-5-carboxamidine;
2-[(3-cyclopropyl-4-methyl-2-oxo-4H-quinazolin-1-yl)methyl]-1-isopentyl-benzimidazole-5-carboxamidine;
2-[(3-cyclopropyl-4,4-dimethyl-2-oxo-quinazolin-1-yl)methyl]-1-isopentyl-benzimidazole-5-carboxamidine;
2-[(3-cyclopropyl-2-oxo-4H-quinazolin-1-yl)methyl]-N′-hydroxy-1-isopentyl-benzimidazole-5-carboxamidine;
2-[(3-cyclopropyl-4-methyl-2-oxo-4H-quinazolin-1-yl)methyl]-N′-hydroxy-1-isopentyl-benzimidazole-5-carboxamidine;
2-[(3-cyclopropyl-4,4-dimethyl-2-oxo-quinazolin-1-yl)methyl]-N′-hydroxy-1-isopentyl-benzimidazole-5-carboxamidine;
2-[(3-cyclopropyl-2-oxo-4H-quinazolin-1-yl)methyl]-1-(4,4,4-trifluorobutyl)benzimidazole-5-carboxamidine;
2-[(3-cyclopropyl-4-methyl-2-oxo-4H-quinazolin-1-yl)methyl]-1-(4,4,4-trifluorobutyl)benzimidazole-5-carboxamidine;
2-[(3-cyclopropyl-4,4-dimethyl-2-oxo-quinazolin-1-yl)methyl]-1-(4,4,4-trifluorobutyl)benzimidazole-5-carboxamidine;
2-[(3-cyclopropyl-4-methyl-2-oxo-4H-quinazolin-1-yl)methyl]-N′-hydroxy-1-(4,4,4-trifluorobutyl)benzimidazole-5-carboxamidine;
2-[(3-cyclopropyl-4,4-dimethyl-2-oxo-quinazolin-1-yl)methyl]-N′-hydroxy-1-(4,4,4-trifluorobutyl)benzimidazole-5-carboxamidine;
1-[[5-(aminomethyl)-1-isopentyl-6-methyl-benzimidazol-2-yl]methyl]-3-cyclopropyl-4H-quinazolin-2-one;
or a pharmaceutically acceptable salt thereof.

10. 1-[[5-(aminomethyl)-1-isopentyl-benzimidazol-2-yl]methyl]-3-cyclopropyl-4H-quinazolin-2-one, or a pharmaceutically acceptable salt thereof.

11. 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

12. A crystalline form of a compound of the formula (I), as defined in claim 1, or a pharmaceutically acceptable salt thereof.

13. A crystalline form of 1-[[5-(aminomethyl)-1-isopentyl-benzimidazol-2-yl]methyl]-3-cyclopropyl-4H-quinazolin-2-one, or a pharmaceutically acceptable salt thereof.

14. A crystalline form of 1-[[5-(aminomethyl)-1-(4,4,4-trifluorobutyl)benzimidazol-2-yl]methyl]-3-cyclopropyl-4H-quinazolin-2-one, or a pharmaceutically acceptable salt thereof.

15. A pharmaceutical composition comprising a compound of formula (I), as defined in claim 1, or a pharmaceutically acceptable salt thereof, and a pharmaceutically acceptable diluent or carrier.

16. A method of treating, or reducing the risk of, RSV which comprises administering to a patient in need thereof a therapeutically effective amount of a compound of formula (I), or a pharmaceutically acceptable salt thereof, as defined in claim 1.

17. A compound of the formula (I), as defined in claim 1, or a pharmaceutically acceptable salt thereof, in combination with one or more further therapeutic agent(s) selected from:
(i) RSV N-protein inhibitors;
(ii) other RSV protein inhibitors (for example those that inhibit the phosphoprotein (P) protein and large (L) protein);
(iii) anti-RSV monoclonal antibodies, such as the F-protein targeting antibodies;
(iv) immunomodulating toll-like receptor compounds;
(v) other respiratory virus anti-virals (for example anti-influenza and anti-rhinovirus compounds); and
(vi) anti-inflammatory compounds,
for use in the treatment of RSV.

18. A compound of the formula (I), as defined in claim 1, or a pharmaceutically acceptable salt thereof, in combination with a RSV N-protein inhibitor for use in the treatment of RSV.

19. A process for the preparation of a compound of formula (I) as defined in claim 1, or a pharmaceutically acceptable salt thereof, which comprises a process (a) or (b):
(a) reacting a compound of formula (II) 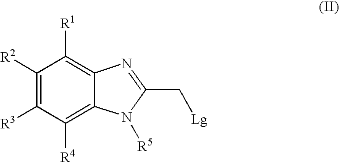
wherein Lg represents a suitable leaving group, R¹, R³, R⁴ and R⁵ are as defined in claim 1 for formula (I) and R² is as defined in claim 1 for formula (I) or a protected derivative thereof, with a compound of formula (III): 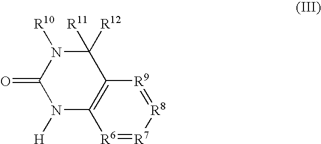
wherein R⁶, R⁷, R⁸, R⁹, R¹⁰, R¹¹ and R¹² are as defined in claim 1 for formula (I); or
(b) when R² represents CH₂NH₂, reduction of a compound of formula (I) wherein R² represents CN: 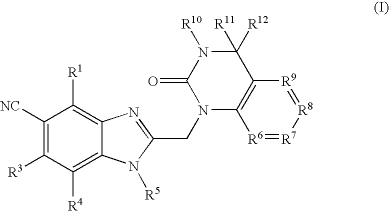
wherein R¹, R³, R⁴, R⁵, R⁶, R⁷, R⁸, R⁹, R¹⁰, R¹¹ and R¹² are as defined in claim 1 for formula (I);
and optionally after (a) or (b) carrying out one or more of the following:
converting the compound obtained to a further compound of the invention
forming a pharmaceutically acceptable salt of the compound
removing any protecting group that is present
preparing a crystalline form thereof.
